(19) United States
(12) Patent Application Publication
(10) Pub. No.: US 2023/0295588 A1
(43) Pub. Date: Sep. 21, 2023

(54) METHODS FOR TREATING INFLAMMATORY BOWEL DISEASE BASED ON HOST-MYCOBIOTA INTERACTIONS

(71) Applicant: Cornell University, Ithaca, NY (US)

(72) Inventors: Iliyan D. Iliev, New York, NY (US); Xin Li, Ithaca, NY (US)

(73) Assignee: Cornell University, Ithaca, NY (US)

(21) Appl. No.: 18/076,795

(22) Filed: Dec. 7, 2022

Related U.S. Application Data
(60) Provisional application No. 63/287,233, filed on Dec. 8, 2021.

(51) Int. Cl. C12N 9/22; G01N 33/569; A61P 31/10; C12N 15/11

(52) U.S. Cl. CPC C12N 9/22; A61P 31/10; C12N 15/11; G01N 33/56961; C12N 2310/20

(57) ABSTRACT

Disclosed herein are methods for selecting a patient suffering from fungal-associated intestinal inflammation for treatment with an IL-1 pathway inhibitor, including inflammasome-blocking drugs based on the presence of candidalysin-secreting C. albicans strains in gut tissue.

### CROSS-REFERENCE TO RELATED APPLICATIONS

This application claims the benefit of and priority to U.S. Provisional Pat. Application No. 63/287,233 filed Dec. 8, 2021, the entire contents of which are incorporated herein by reference.

### SEQUENCE LISTING

The instant application contains a Sequence Listing which has been submitted electronically in XML format and is hereby incorporated by reference in its entirety. Said XML copy, created on May 21, 2023, is named 093873-1391_SL.xml and is 49,663 bytes in size.

### GOVERNMENT SUPPORT

This invention was made with government support under DK113136 and DK121977 awarded by the National Institutes of Health. The government has certain rights in the invention.

### TECHNICAL FIELD

Disclosed herein are methods for selecting a patient suffering from fungal-associated intestinal inflammation for treatment with an IL-1 pathway inhibitor, including inflammasome-blocking drugs based on the presence of candidalysin-secreting C. albicans strains in gut tissue.

### BACKGROUND

The following description of the background of the present technology is provided simply as an aid in understanding the present technology and is not admitted to describe or constitute prior art to the present technology.

The fungal microbiota (mycobiota) is an integral part of the complex multi-kingdom microbial community colonizing the mammalian gastrointestinal tract and plays an important role in immune regulation¹⁻⁶. Deep sequencing-based surveys of the gut mycobiome in several disease cohorts provide consistent evidence for “fungal dysbiosis” as a hallmark³⁻⁹ of inflammatory bowel disease (IBD), the most prevalent forms of which are Crohn’s disease (CD) and ulcerative colitis (UC), which affects up to 3.5 million individuals¹⁰. Antibodies against fungal mannan (ASCA), have been routinely used as serological biomarkers to define IBD subtypes and patient responses to therapies, further linking fungi with intestinal inflammation¹¹.

Candida is the most prevalent fungal genus and its relative abundance is consistently increased in several IBD cohorts based on fecal sequencing^{3,6,12}. Notably C. albicans can also act as an immunogen for ASCA induction^{13,14}. Experimental studies demonstrate that Candida species associated with the intestinal mucosa are sensed by gut resident-macrophages and thus have the potential to induce protective immune responses or trigger inflammation in a context-dependent manner¹⁵. Despite this evidence, it is currently unknown whether fungal signatures captured by deep sequencing represent living organisms and whether specific fungi have functional consequences for disease development in affected individuals. Consistently, lack of association between changes in mycobiota composition and disease severity have been observed in IBD cohorts, despite a consistent increase in Candida species^{3,5,6,12}.

Thus, there is a need for methods for determining which fungi in the human intestinal mucosa play an essential role in directing mucosal immunity or disease outcomes.

### SUMMARY OF THE PRESENT TECHNOLOGY

In one aspect, the present disclosure provides a method for treating a patient suffering from a fungal-associated intestinal inflammatory disorder comprising administering to the patient an effective amount of an IL-1 pathway inhibitor, wherein gut tissue of the patient comprises a population of candidalysin-secreting C. albicans.

In one aspect, the present disclosure provides a method for selecting a patient suffering from a fungal-associated intestinal inflammatory disorder for treatment with an IL-1 pathway inhibitor comprising (a) detecting the presence of candidalysin-secreting C. albicans in a biological sample obtained from the patient; and (b) administering to the patient an effective amount of an IL-1 pathway inhibitor. In some embodiments, the biological sample is a colonic mucosa-enriched lavage sample, a fecal sample, a rectal swab, or an intestinal sample.

Additionally or alternatively, in some embodiments of the methods disclosed herein, the fungal-associated intestinal inflammatory disorder is inflammatory bowel disease (IBD), Crohn’s disease (CD), or ulcerative colitis (UC).

In any and all embodiments of the methods disclosed herein, the IL-1 pathway inhibitor is an inflammasome-blocking drug, an anti-IL-1R1 antibody or antigen binding fragment, Anakinra, Rilonacept, Canakinumab, Gevokizumab, LY2189102, MABp1, MEDI-8968, CYT013, sIL-1RI, sIL-1RII, EBI-005, CMPX-1023, MCC950, Inzomelid, Somalix, NT-0167, IFM-2427 (DFV890), Dapansutrile (OLT1177), glyburide, 16673-34-0, JC124, FC11A-2, parthenolide, Bay 11-7082, BHB, MNS, CY-09, tranilast, oridonin, VX-740, or VX-765.

Additionally or alternatively, in some embodiments of the methods disclosed herein, the candidalysin-secreting C. albicans expresses elevated enhanced filamentous growth protein 1 (EFG1) expression compared to a reference non-filamentous C. albicans strain or a predetermined threshold. In certain embodiments, the candidalysin-secreting C. albicans expresses increased hyphae production relative to a reference non-filamentous C. albicans strain. Additionally or alternatively, in certain embodiments of the methods disclosed herein, the candidalysin-secreting C. albicans expresses elevated expression levels of at least one protease selected from among SAP6, SAP5, or SAP2 compared to a reference non-filamentous C. albicans strain or a predetermined threshold. In other embodiments of the methods disclosed herein, the candidalysin-secreting C. albicans expresses elevated expression levels of ALS3 or ALS1 compared to a reference non-filamentous C. albicans strain or a predetermined threshold. In any of the preceding embodiments of the methods disclosed herein, the reference non-filamentous C. albicans strain is an efg1Δ/Δ C. albicans mutant strain.

Additionally or alternatively, in some embodiments of the methods disclosed herein, the candidalysin-secreting C. albicans induces an in vivo proinflammatory response in host cells. In some embodiments, the in vivo proinflammatory responses comprise neutrophil infiltration and/or Th17 responses in the colon of the patient.

In certain embodiments, the human subject is diagnosed with or is suffering from inflammatory bowel disease (IBD), Crohn’s disease (CD), or ulcerative colitis (UC).

Additionally or alternatively, in some embodiments, the at least one C. albicans strain inflicts macrophage damage that is elevated or comparable to C. albicans SC5314. In some embodiments, the at least one C. albicans strain comprises one or more of IDD581, IDA653, IDB311, IDB671, IDB312, IDB313, IDB831, IDB071, IDB072, IDB101, IDB104, IDC481, IDC482, IDC483, IDC571, IDC572, IDD582, IDC711, or IDC712.

In another aspect, the present disclosure provides a kit comprising (a) a first expression vector comprising a nucleic acid sequence encoding a Candida-compatible Cas9 nuclease and a nucleic acid sequence encoding a synthetic guide RNA (sgRNA) that is configured to cleave a region in a target gene of at least one C. albicans strain that resides in human gut tissue, wherein the target gene is associated with high immune cell-damaging capacity and wherein the at least one C. albicans strain induces proinflammatory immunity in a human subject; and (b) a heterologous repair template nucleic acid sequence comprising (i) a 5′ region that is homologous to a C. albicans nucleic acid sequence that is upstream or downstream from the region in the target gene that is cleaved by the sgRNA and (ii) a 3′ region comprising an open reading frame (ORF) deletion of the target gene, wherein the target gene is ECE1, UME6, or FLO8. In some embodiments, the kit further comprises a second expression vector comprising a nucleic acid sequence encoding a Candida-compatible Cas9 nuclease and a nucleic acid sequence encoding a synthetic guide RNA (sgRNA) that is configured to cleave a region in EFG1, or CPH1 of the at least one C. albicans strain. Additionally or alternatively, in some embodiments, the 5′ region of the heterologous repair template nucleic acid sequence is about 60 base pairs in length. In other embodiments, the 3′ region of the heterologous repair template nucleic acid sequence is about 20 base pairs in length.

### BRIEF DESCRIPTION OF THE DRAWINGS

FIGS. 1A-1H. C. albicans expands in UC patient colonic mucosa and promotes inflammation in a murine model of colitis. FIG. 1A, Principal coordinate analysis (PCoA) plot of distance ordination for fungal ITS1 OTUs in colonic mucosa (MUC) enriched samples from non-IBD (non-IBD, n=38) or ulcerative colitis-affected (UC, n=40) individuals. Based on quality control, one non-IBD sample was excluded from further mycobiome sequencing and analysis. Analysis of similarities (ANOSIM) statistics: R²=0. 0.3735, P=0.001. FIG. 1B, Relative abundance of detected fungal genera. FIG. 1C, Relative abundance of Candida spp., Saccharomyces spp. and other less represented fungal genera in the human mucosa (“others”); Mann-Whitney (non-paired) test, Benjamini-Hochberg (BH) corrected. FIG. 1D, Live C. albicans dominated the colonic mucosa of UC patients. Isolated fungal colonies from each individual subject were identified by MALDI-TOF, and viable C. albicans colony forming units (cfu) per mL of lavage sample were determined. FIGS. 1E-1H, Colitis was induced in WT SPF mice that were pretreated (Pred+PBS) or not (Ctrl+PBS) with prednisolone. A group of DSS-treated mice was fed with C. albicans under prednisolone therapy (Pred+C.a). FIGS. 1E-1F, H&E staining and histology score of colon sections performed upon sacrifice determined increased disease severity in C. albicans (Pred+C.a) colonized mice. FIGS. 1G-1H, Representative flow cytometry plots and quantification of the frequency and total cell number of colonic lamina propria (cLP) CD4⁺ T cells (FIG. 1G, from left to right) and CD11b⁺Ly6G⁺ neutrophils (FIG. 1H, from left to right), n=5 in each group (FIGS. 1G-1H). Results are shown as mean ± s.e.m. Each dot represents an individual mouse. Data are representative of two or three independent experiments. *P < 0.05, **P < 0.01, ***P < 0.001, unpaired two-tailed Student’s t-test (FIGS. 1C-1D) or one-way ANOVA followed by the Tukey’s post hoc test (FIGS. 1F-1H).

FIGS. 2A-2N. Cell damage and proinflammatory immunity induced by human gut-derived C. albicans is strain-dependent. FIG. 2A, Murine bone marrow-derived macrophages (mBMDM) were co-cultured with human gut-derived C. albicans isolates (three isolates per human individual; MOI=5) for 16 hours. Cell cytotoxicity was measured by the lactate dehydrogenase (LDH) release assay. The cytotoxicity of each isolate was compared to laboratory C. albicans strain SC5314. Data are representative of three independent experiments, unpaired two-tailed Student’s t-test. FIG. 2B, Filamentation phenotype of each isolate was determined in a Spider agar assay and compared the capacity of C. albicans isolates to induce damage of mBMDM (LDH assay) based on different filamentation phenotype were compared. FIG. 2C, Representative images of low-damaging C. albicans strain (LD/C.a; IDC561) that is defective in filamentation and high-damaging C. albicans strain (HD/C.a; IDB311) that is able to form filamentation. FIGS. 2D-2F, WT germ-free (GF) mice were mono-colonized with PBS (n=6), LD/C.a (IDC561, n=6) or HD/C.a (IDB311, n=6) strains for three weeks. FIG. 2D, Fecal C. albicans burden was measured at day 21. FIGS. 2E-2F, Representative flow cytometry plots and quantification of CD11b⁺Ly6G⁺ neutrophils (FIG. 2E, from left to right) and CD4⁺IL-17A⁺ Th17 cells (FIG. 2F, from left to right) in the colon. FIGS. 2G-2I, ASF mice were colonized with PBS (n=6), LD/C.a (IDC561, n=6) or HD/C.a (IDB311, n=6) for three weeks. FIGS. 2G, C. albicans burden in the feces at day 21. FIG. 2H, Frequency and total numbers of colonic CD4⁺IL-17A⁺ Th17 cells (from left to right). FIG. 2I, Frequency of colonic CD4⁺RORγt⁺ cells. FIGS. 2J-2K, ASF mice were colonized with LD/C.a (IDB891, n=5) or HD/C.a (IDB101, n=6) for three weeks. FIG. 2J, C. albicans burden in the feces at day 21. FIG. 2K, Frequency of CD4⁺IL-17A⁺ Th17 cells in the colon were assessed. FIGS. 2L-2N, Mice colonized PBS (n=10), HD/C.a IDB311 (n=9) and LD/C.a IDC561 (n=7) were treated with prednisolone followed by DSS-mediated induction of colitis. FIG. 2L, Fecal C. albicans burdens were assessed upon sacrifice. FIG. 2M, Colon length. FIG. 2N, Frequency of CD11b⁺Ly6G⁺ neutrophils in the colon was assessed by flow cytometry (FIG. 2N). Results are shown as mean ± s.e.m. Each dot represents an individual mouse. Data are representative of two independent experiments. *P < 0.05, **P < 0.01, ***P < 0.001, unpaired two-tailed Student’s t-test (FIGS. 2B, 2D, 2G- 2L) or one-way ANOVA followed by the Tukey’s post hoc test (FIGS. 2E-2F, and FIGS. 2M-2N).

FIGS. 3A-3M. HD C. albicans promotes intestinal pro-inflammatory immunity through the Efg1-Ece1-dependent factor Candidalysin. FIG. 3A, Schematic view of CRISPR/Cas9-mediated mutagenesis in C. albicans isolates as described in Methods. FIGS. 3B-3C, Caco2 cells were infected with live C. albicans for 12 hours, and both the EFG1 expression of C. albicans (FIG. 3B) and the LDH release from Caco2 cells (FIG. 3C) were assessed. HD and LD C. a strains, and respective eƒg1Δ/Δ (efg1) strains were used in this experiment. Results are shown as mean ± s.d. Data are representative of three independent experiments. FIGS. 3D-3F, GF mice were colonized with PBS (GF, n=5), HD/C.a IDB311 (HD, n=6) or HD/C.a IDB311 efg1 (HD efg1, n=6) strains for three weeks. FIG. 3D, Fecal C. albicans burdens were measured at day 21. Frequency and total cell numbers of IL-17A⁺CD4⁺ T cell (FIG. 3E) and IL-17A⁺IL-17F⁺CD4⁺ T cell (FIG. 3F) in the colon were assessed by flow cytometry. FIG. 3G, GF mice were colonized with HD/C.a IDB311 (HD, n=10), LD/C.a IDC561 (LD, n=8) strains for three weeks. FISH-stained C. albicans yeast and hyphae in the colons of HD/C.a or LD/C.a strain colonized groups. Right, an image with DAPI-stained colonic epithelial cell (blue), FITC-UEA-1-stained mucus layers (green), and rRNA Cy3-probe-stained C. albicans (Red). Left, C. albicans morphotypes. Scale bar = 25 µm. FIGS. 3H-3I, LDH release were assessed from Caco2 cells infected with C. albicans for 12 hours. FIG. 3H, HD/C.a IDB311 and LD/C.a IDC561 strains, and respective ECE1 mutant strains. FIG. 3I, HD/C.a IDB101, LD/C.a IDB891 and respective ECE1 mutant strains. FIGS. 3J-3M, GF mice were colonized with PBS (GF, n=8), HD/C.a IDB311 (HD, n=10), LD/C.a IDC561 (LD, n=8) and HD ece1 (n=10) or LD ece1 (n=10) strains for three weeks. FIGS. 3J-3L, Representative flow cytometry plots (FIG. 3J), and quantification of the frequency and total cell numbers of IL-17A⁺CD4⁺ T cells (FIG. 3K) and IL-17A⁺IL-17F⁺CD4⁺ T cells (FIG. 3L) in the colon. FIG. 3M, Fecal C. albicans burden were measured at day 21. Each dot represents an individual mouse. Results are shown as mean ± s.e.m. All data are representative of at least two independent experiments. *P < 0.05, **P < 0.01, ***P < 0.001 and ****P < 0.0001, unpaired two-tailed Student’s t-test (FIG. 3D) or one-way ANOVA followed by the Tukey’s post hoc test (FIGS. 3E-3F and 3K, 3L, 3M).

FIGS. 4A-4C. C. albicans displays high strain diversity across individuals; Clonal expansion, and microevolution occur in the human gut. FIG. 4A, Dendrogram based on whole genome sequence similarity of 18 newly sequenced human gut C. albicans isolates, each obtained from an individual subject, together with 94 C. albicans strains (Supplementary Table C) collected worldwide⁵¹. Strains isolated from non-IBD subjects (blue, nIBD), UC patients (red), or other isolates collected worldwide (gray) are color labeled. Clade labels (roman numerals and letters) are shown as defined in Ropars et al.⁵¹ for the samples shown in gray and are inferred based on the sequence similarity for the gut isolates, when possible (otherwise labelled “NC”). FIG. 4B, Dendrogram showing genome-wide SNP-based distances among two or three human gut C. albicans isolates obtained from the same individual subject. Strains isolated from non-IBD subjects (nIBD), UC patients (UC), and refence strain C. albicans SC5314 (Ref. Strain). FIG. 4C, Heatmap showing genome-wide density of heterozygous SNPs from two or three human gut C. albicans isolates obtained from the same individual subject. Strains isolated from non-IBD subjects (nIBD), UC patients (UC), and refence strain C. albicans SC5314 (Ref. Strain) are labeled. Color density indicates the number of heterozygous SNPs detected in each 10 kbp window of an isolate’s genome. Arrows point to the genomic locations of the ECE1 and EFG1 genes.

FIGS. 5A-5L. Capacity of patient-specific C. albicans strains to induce IL-1β reflects disease severity in UC. FIGS. 5A-5C, TNFα, IL-6 and IL-1β release in culture supernatants of unprimed (FIGS. 5A-5B) or LPS-primed (FIG. 5C) human monocyte-derived macrophages (hMDMs) after incubation with live gut derived-C. albicans isolates (MOI=5). Cytokine release was measured by ELISA. hMDM damage measured (LDH assay) in the same experiment was correlated with specific cytokine release (FIGS. 5A-5C). FIG. 5D, Correlation between hMDM damage by patient-specific gut C. albicans and UC-severity score (Mayo score) in corresponding UC patients (n=10) is depicted. FIG. 5E, Correlation between the proinflammatory cytokine IL-1β production in hMDMs incubated with patient-specific gut C. albicans and UC-severity score (Mayo score) in the respective patients (n=10) is depicted. FIG. 5F, Correlation between the relative abundance of Candida of UC patient and UC-severity score. FIGS. 5A-5E, Linear regression analysis and P-values are shown in each panel. FIGS. 5G-5L, Mice colonized with or without HD/C.a IBD311 were treated with prednisolone (Pred) followed by DSS-mediated induction of colitis. Each mouse further treated with 1 mg anti-IL-1R1 IgG (αIL1R) or isotype IgG (IgG) at the time point indicated in the schematic figureof experimental layout. n=8 (IgG+PBS), n=9 (IgG+C.a) and n=7 (αIL-1R1+C.a) (FIG. 5G). FIG. 5H, Colon length, FIGS. 5I-L, cLP CD11b⁺Ly6G⁺ neutrophils (FIG. 5I), and IL-17A⁺IL-17F⁺CD4⁺ T cells (FIGS. 5J, 5L), and IL-17A⁺IFNγ⁺CD4⁺ T cells (FIGS. 5K, 5L) cell frequencies in the colon were assessed by flow cytometry. Results are shown as mean ± s.e.m. Each dot represents an individual mouse. All data are representative of at least two independent experiments. *P < 0.05, ***P < 0.001, ****P < 0.0001, one-way ANOVA followed by the Tukey’s post hoc test (FIGS. 5H-5I and 5L). Each dot represents an individual, and bars represent mean. One-way ANOVA followed by the Tukey’s post hoc test.

FIGS. 6A-6C. Relative abundance of intestinal fungal genera in non-IBD and patients with UC. FIG. 6A, Alpha diversity analysis was analyzed using the Shannon diversity index among fungi communities at the fungal OTU level in colonic mucosa (MUC) enriched samples from non-IBD (non-IBD, n=38) or ulcerative colitis-affected (UC, n=40) individuals. Based on quality control one non-IBD sample was excluded from further mycobiome sequencing and analysis. FIG. 6B, Non-metric multidimensional scaling (NMDS) plot of distance ordination based on Bray-Curtis dissimilarities for fungal ITS1 OTUs in colonic mucosa (MUC) enriched samples from non-IBD or ulcerative colitis-affected (UC) individuals. Analysis of similarities (ANOSIM) statistics: R²=0.3735, P=0.001. FIG. 6C, Relative genus abundance of intestinal fungal genera. Each dot represents an individual human subject, analysis performed with Mann Whitney test followed by Benjamini-Hochberg (BH) correction.

FIGS. 7A-7F. Intestinal colonization by C. albicans does not cause spontaneous colitis during homeostasis nor does it aggravate D55-induced colitis. FIG. 7A, Fecal C. albicans burdens were assessed after 3 days of C. albicans colonization in mice that received either control feeding water (n=4) or DSS water (n=5) for 4 days. Dots represent individual mice. FIGS. 7A-7F, Mice, after being gavaged with PBS (n=5) or C. albicans (n=5) for 14 days, were induced by 3% DSS water for 7 days. FIG. 7B, Schematic figure of DSS-induced colitis model of mice with intestinal colonization by C. albicans. FIG. 7C, H&E straining (left) and histology score (right) of colon section. FIGS. 7D-7E, Representative flow cytometry plot and quantification of Foxp3⁺ (FIG. 7D) and RORγt⁺ CD4⁺ T (FIG. 7E) cells. FIGS. 7A-7F, Mice were gavaged twice per week with PBS (n=5) or C. albicans WT SC5314 (n=5). Mice were sacrificed four months later for colon length (FIG. 7F) and histology evaluation (FIG. 7F). Results are shown as mean ± s.e.m. Each dot represents an individual mouse. Data are representative of two independent experiments. *P < 0.05, **P < 0.01, unpaired two-tailed Student’s t-test.

FIGS. 8A-8E. C. albicans expands and promotes intestinal inflammation under immunosuppression therapy for UC. FIG. 8A, Medication data summary for UC patients (n=5609) who visited New York-Presbyterian Hospital from 2016-2018. Corticosteroids (CS, n=2522); Others include Mercaptopurine, Azathioprine, Methotrexate, Tacrolimus, Cyclosporine and Biologics (n=3057). FIG. 8B, Fecal C. albicans burdens were measured after 3 days of C. albicans colonization in mice that received either PBS (n=5) or prednisolone daily (10 mg/kg/day, n=5) . FIGS. 8C-8E, WT SPF mice were fed PBS (n=5), Pichia kudriavzevii (P.k, n=5), or Candida albicans (C.a, n=5) while receiving prednisolone treatment (Pred+PBS, Pred+P.k, or Pred+C.a). 3% DSS drinking water was used to induce colitis for 7 days. Mice were sacrificed three days after the DSS water was removed. n=6 for each group. c, Fecal fungal burdens upon sacrifice. FIG. 8D, Colon length was assessed. FIG. 8E, Representative flow cytometry plots and quantification of the frequency IL-17A⁺CD4⁺ T cells in the colons. Results are shown as mean ± s.e.m. Each dot represents an individual mouse. Data are representative of two or three independent experiments. *P < 0.05, **P < 0.01, unpaired two-tailed Student’s t-test (FIGS. 8B-8C) or one-way ANOVA followed by the Tukey’s post hoc test (FIGS. 8D-8E).

FIGS. 9A-9I. Gut-derived C. albicans isolates exhibit different phenotypic responses in a filamentation assay. FIG. 9A, Gut C. albicans isolates were cultured on Spider agar at 37° C. for 5 days followed by assessment of the edge of wrinkled and smooth colonies. Percentage of the filamentation phenotypes of gut C. albicans isolates in FIG. 2A on spider agar. FIGS. 9B-9I, Filamentation phenotype of representative gut C. albicans isolates used in FIG. 2A. FIGS. 9B, C. albicans IDB311, IDB312, and IDB313 isolates from UC patient IDB31. FIGS. 9CC. albicans IDB831, IDB832, and IDB833 isolates from UC patient IDB83. FIGS. 9D, C. albicans IDB101, IDB102, and IDB104 isolates from UC patient IDB10. FIGS. 9E, C. albicans IDC481, IDC482, and IDC483 isolates from UC patient IDC48. FIGS. 9F, C. albicans IDB071, IDB072, and IDB073 isolates from UC patient IDB07. FIGS. 9G, C. albicans IDA651, IDA652, and IDA653 isolates from UC patient IDA65. FIGS. 9H, C. albicans IDA921, IDA922, and IDA923 isolates from UC patient IDA92. FIGS. 9I, C. albicans IDC561, IDC562, and IDC563 isolates from non-IBD individual IDC56.

FIGS. 10A-10E. High-damaging strains induce greater proinflammatory immune responses. FIGS. 10A-10C, WT germ-free (GF) mice were monocolonized with LD/C.a. (IDC561) or HD/C.a. (IDB311) strains for three weeks, n=6 mice per group. FIG. 10A, Gating strategy to analyze CD4⁺ T cells in colonic lamina propria cells. Representative flow cytometry plots and quantification of CD4⁺IL-17A⁺IL-17F⁺ (FIG. 10B, from left to right) and CD4⁺RORγt⁺ cells (FIG. 10C, from left to right) in the colon. FIGS. 10D-10E, ASF mice were colonized with LD/C.a (IDC662, n=6) or HD/C.a (IDC711, n=6) for three weeks. FIGS. 10D, C. albicans burden in the feces at day 21. FIG. 10E, Frequency of CD4⁺IL-17A⁺ (left) and frequency of CD4⁺IL-17A⁺IL-17F⁺ in the colon were assessed. Results are shown as mean ± s.e.m. Each dot represents an individual mouse. Data are representative of two independent experiments. *P < 0.05, **P < 0.01, ***P < 0.001, ****P < 0.0001, unpaired two-tailed Student’s t-test (FIGS. 10D-10E) or one-way ANOVA followed by the Tukey’s post hoc test (FIGS. 10B-10C).

FIGS. 11A-11E. EFG1-dependent candidalysin is required for cell damage in high-damaging C. albicans strains from the human gut. FIG. 11A, Dendrogram showing SNP-based distances between EFG1 sequences of isolates. Each isolate was obtained from an individual subject from the non-IBD or UC patient group. FIG. 11B, Caco2 cells were infected with live C. albicans (MOI=1) for 12 hours and LDH release of Caco2 cells was assessed. HD/C.a IDB101, HD/C.a IDB101 eƒg1Δ/Δ mutant (efg1), LD/C.a IDB891, and LD/C.a IDB891 eƒg1Δ/Δ mutant (efg1) were used in this experiment.. Mutant strains were genetically edited by CRISPR/Cas9 method. Results are shown as mean ± s.d. Data are representative of three independent experiments. FIG. 11D, C. albicans HD/C.a IDB311, HD/C.a IDB101 and the respective EFG1 mutants were cultured on Spider agar at 37° C. for 5 days followed by assessment of the filamentation phenotype. FIG. 11D, ECE1 gene expression in C. albicans cells were assessed upon EFG1 gene deletion. FIG. 11D, Log2 transformed ratio of gene expression between EFG1 mutant and WT C. albicans strain upon colonization of the large intestine represented as a volcano plot. Data were obtained and analyzed from a recent resource dataset from Witchley et al. 2021.

FIGS. 12A-12C. Genomic comparative analysis of genetic polymorphisms in CPH1, UME6 and FLO8 among high- and low-damaging strains. FIGS. 12A-12C, Dendrogram showing SNP-based distances between CPH1 (FIG. 12A), UME6 (FIG. 12B) and FLO8 (FIG. 12C) sequences of isolates. Each isolate was obtained from an individual subject from the non-IBD or UC patient group.

FIG. 13. HD and LD C. albicans strains both colonize the intestine and form a mixture of yeast and hyphal morphotypes. Fluorescence in situ hybridization (FISH) was utilized to visualize the morphology of C. albicans IDB311 (HD/C.a IDB311), IDC561(LD/C.a IDC561), and respective ECE1 mutant strains (HD/C.a IDB311 ece1Δ/Δ and LD/C.a IDC561 ece1Δ/Δ) in the colon tissue after 21 days of mono-colonization of C. albicans. The nuclei of colonic epithelial cells were stained with DAPI (blue), colonic mucin was stained with a FITC-conjugated Ulex europaeus agglutinin (UEA-I, lectin) (green), and C. albicans was stained with a Cy3-coupled pan-fungal-specific probe (red). The colon tissue of germ-free mice was used as a control. The scale bar indicates 25 µm. Colon sections from mice colonized with C. albicans strains. n=6 for each group, n represents an individual mouse. Data are repeated of two independent experiments.

FIGS. 14A-14J. Gut C. albicans promotes intestinal pro-inflammatory immunity through Candidalysin. FIGS. 14A-14B, Cell damage by C. albicans. mBMDM (FIG. 14A) and Caco2 cells (FIG. 14B) were incubated with live C. albicans (MOI=5) wild-type parental (C.a Pare) or C. albicans ece1Δ/Δ (C.a ecel) strains for 16 hours and LDH release in the supernatants was measured. Results are shown as mean ± s.d. Data are representative of three independent experiments. FIGS. 14C-14E, ASF mice were colonized with C.a. Pare or C.a ecel strains for three weeks. n=6 mice in each group. FIGS. 14C-14D, Frequency and total cell numbers of CD11b⁺Ly6G⁺ neutrophils (FIG. 14C, from left to right) and IL-17A⁺CD4⁺ T cells (FIG. 14D, from left to right) in the colon were assessed by flow cytometry. FIG. 14E, Fecal C. albicans burdens were measured at day 21. FIGS. 14F-14J, WT SPF mice were colonized with or without C.a Pare or C.a ece1 strain, DSS colitis was induced followed by treatment with prednisolone. FIG. 14F, Fecal C. albicans burden were measured upon sacrifice. FIG. 14G, representative H&E colon section. FIG. 14H, Histology scores. FIG. 14I, Frequency of cLP neutrophils. FIG. 14J, IL-17A⁺CD4⁺ T cells were assessed upon sacrifice. Results are shown as mean ± s.e.m. Each dot represents an individual mouse. n=5 mice in each group (FIGS. 14I-14J). All data are representative of at least two independent experiments. *P < 0.05, **P < 0.01, ***P < 0.001 and ****P < 0.0001, unpaired two-tailed Student’s t-test (FIGS. 14A-14B, and 14E-14F) or one-way ANOVA followed by the Tukey’s post hoc test (FIGS. 14C-14D, and FIGS. 14H-14J).

FIGS. 15A-15B. Genomic comparative analysis of ECE1 among high- and low-damaging strains show lack of correlation between damaging potential and genetic polymorphisms in these genes. FIG. 15A, Dendrogram showing SNP-based distances between ECE1 sequences of isolates. Each isolate was obtained from an individual subject from the non-IBD or UC patient group. FIG. 15B, Multiple sequence alignment of candidalysin (SK1 peptide) amino acid sequences across multiple isolates of C. albicans as shown in FIG. 15A. Four isoforms of candidalysin across strains show no association/clustering of specific isoform with HD or LD strain. Two haplotypes are shown for isolates that are heterozygous in this region. FIG. 15B discloses SEQ ID NOS 33, 33, 33, 33, 33, 33, 33, 33, 33, 33, 33, 33, 33, 34, 34, 34, 35, 35, 35, 35, 35, 35, 35, 35, 35, 35, 35, 35, 36, 36, respectively, in order of appearance.

FIGS. 16A-16J. candidalysin contributes to the secretion of IL-1β by macrophages. FIG. 16A, LDH release measured in culture supernatants of hMDM after infection with C. albicans parental strain (Pare), C.a ece1Δ/ece1/Δ (C.a ece1) and an untreated group (Ctrl) for 16 hours. FIGS. 16B-16I, Macrophage-released mediators measured by cytometric bead assays from cultures in FIG. 16A. FIG. 16J, IL-1β release measured in culture supernatants of LPS-primed hMDMs infected with C. albicans parental strain, C.a ece1, and in uninfected group. Parallel experiments were performed with FIG. 11A. Results are shown as mean ± s.d. All data are representative of three independent experiments. *P < 0.05, **P < 0.01, ***P < 0.001, one-way ANOVA followed by the Tukey’s post hoc test.

FIGS. 17A-17B. Immune mediators released by macrophage upon infection with gut -derived C. albicans strains. FIGS. 17A-17B, TNFα (FIG. 17A) and IL-6 (FIG. 17B) release in culture supernatants of unprimed human monocyte-derived macrophages (hMDMs) after incubation with live gut derived-C. albicans isolates (MOI=5). Cytokine release measured by ELISA. Correlation between TNFα and IL-6 cytokine from hMDM induced by patient-specific gut C. albicans and UC-severity score (Mayo score) in corresponding UC patients (n=10) is depicted. Linear regression analysis and P-values are shown in each panel.

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir’s Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

Disclosed herein is a translational platform for the functional exploration of the mycobiome at a fungal strain- and patient-specific level. The application of this platform to mucosal samples collected from healthy and IBD patients, allowed the identification of a rich C. albicans strain-level diversity in the human gut and the unexpected domination of strains with high cell-damaging capacity and immunoreactivity in the gut of specific patients.

Combining high-resolution mycobiota-sequencing, fungal culture-based identification, fungal genomics, a CRISPR/Cas9-based fungal strain editing system, in vitro functional immunoreactivity assays and in vivo models, this platform allows for the exploration of host-fungal immune crosstalk within the human gut. We discovered a rich genetic diversity of opportunistic Candida albicans strains that dominated the colonic mucosa of ulcerative colitis (UC) patients. Among these human gut-derived isolates, strains with high immune cell-damaging capacity (HD strains) reflect disease features of individual UC patients and aggravated intestinal inflammation in vivo through IL-1β-dependent mechanisms. Niche-specific inflammatory immunity by HD strains in the gut was dependent upon the C. albicans secreted peptide toxin Candidalysin during the transition from a benign commensal to a pathobiont state. These findings unveil the strain-dependent nature of host-fungal interactions in the human gut and highlight new diagnostic and therapeutic targets for diseases of inflammatory origin.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

The term “adapter” refers to a short, chemically synthesized, nucleic acid sequence which can be used to ligate to the end of a nucleic acid sequence in order to facilitate attachment to another molecule. The adapter can be single-stranded or double-stranded. An adapter can incorporate a short (typically less than 50 base pairs) sequence useful for PCR amplification or sequencing.

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, or topically. Administration includes self-administration and the administration by another.

As used herein, the terms “amplify” or “amplification” with respect to nucleic acid sequences, refer to methods that increase the representation of a population of nucleic acid sequences in a sample. Nucleic acid amplification methods are well known to the skilled artisan and include ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), recombinase- polymerase amplification (RPA)(TwistDx, Cambridge, UK), transcription mediated amplification, signal mediated amplification of RNA technology, loop-mediated isothermal amplification of DNA, helicase-dependent amplification, single primer isothermal amplification, and self- sustained sequence replication (3SR), including multiplex versions or combinations thereof. Copies of a particular nucleic acid sequence generated in vitro in an amplification reaction are called “amplicons” or “amplification products.”

The term “Cas9” or “Cas9 nuclease” refers to an RNA-guided nuclease comprising a Cas9 protein, or a fragment thereof (e.g., a protein comprising an active, inactive, or partially active DNA cleavage domain of Cas9, and/or the gRNA binding domain of Cas9). A Cas9 nuclease is also referred to sometimes as a casn1 nuclease or a CRISPR (clustered regularly interspaced short palindromic repeat)-associated nuclease. CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species. See, e.g., Jinek M., Chylinski K., Fonfara I., Hauer M., Doudna J. A., Charpentier E. Science 337:816-821(2012), the entire contents of which is hereby incorporated by reference. Cas9 recognizes a short motif in the CRISPR repeat sequences (the PAM or protospacer adjacent motif) to help distinguish self versus non-self. Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference). Cas9 orthologs have been described in various species, including, but not limited to, S. pyogenes and S. thermophilus. In some embodiments, a Cas9 nuclease has an inactive (e.g., an inactivated) DNA cleavage domain, that is, the Cas9 is a nickase.

A nuclease-defective Cas9 protein may interchangeably be referred to as a “dCas9″ protein (for nuclease-“dead” Cas9). Methods for generating a Cas9 protein (or a fragment thereof) having an inactive DNA cleavage domain are known (See, e.g., Jinek et al., Science. 337:816-821(2012); Qi et al., “Repurposing CRISPR as an RNA-Guided Platform for Sequence-Specific Control of Gene Expression” (2013) Cell. 28; 152(5):1173-83, the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al., Science. 337:816-821(2012); Qi et al., Cell. 28; 152(5):1173-83 (2013)). In some embodiments, proteins comprising fragments of Cas9 are provided. For example, in some embodiments, a protein comprises one or two Cas9 domains: (1) the gRNA binding domain of Cas9; or (2) the DNA cleavage domain of Cas9.

The terms “complementary” or “complementarity” as used herein with reference to polynucleotides (i.e., a sequence of nucleotides such as an oligonucleotide or a target nucleic acid) refer to the base-pairing rules. The complement of a nucleic acid sequence as used herein refers to an oligonucleotide which, when aligned with the nucleic acid sequence such that the 5′ end of one sequence is paired with the 3′ end of the other, is in “antiparallel association.” For example, the sequence “5′-A-G-T-3”′ is complementary to the sequence “3′-T-C-A-5.” Certain bases not commonly found in naturally-occurring nucleic acids may be included in the nucleic acids described herein. These include, for example, inosine, 7-deazaguanine, Locked Nucleic Acids (LNA), and Peptide Nucleic Acids (PNA). Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complement sequence can also be an RNA sequence complementary to the DNA sequence or its complement sequence, and can also be a cDNA.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

“Detecting” as used herein refers to determining the presence of a polynucleotide or polypeptide of interest in a sample. Detection does not require the method to provide 100% sensitivity. Analysis of nucleic acid markers can be performed using techniques known in the art including, but not limited to, sequence analysis, and electrophoretic analysis. Non-limiting examples of sequence analysis include Maxam-Gilbert sequencing, Sanger sequencing, capillary array DNA sequencing, thermal cycle sequencing (Sears et al., Biotechniques, 13:626-633 (1992)), solid-phase sequencing (Zimmerman et al., Methods Mol. Cell Biol, 3:39-42 (1992)), sequencing with mass spectrometry such as matrix-assisted laser desorption/ionization time-of-flight mass spectrometry (MALDI-TOF/MS; Fu et al., Nat. Biotechnol, 16:381-384 (1998)), and sequencing by hybridization. Chee et al., Science, 274:610-614 (1996); Drmanac et al., Science, 260:1649-1652 (1993); Drmanac et al., Nat. Biotechnol, 16:54-58 (1998). Non-limiting examples of electrophoretic analysis include slab gel electrophoresis such as agarose or polyacrylamide gel electrophoresis, capillary electrophoresis, and denaturing gradient gel electrophoresis. Additionally, next generation sequencing methods can be performed using commercially available kits and instruments from companies such as the Life Technologies/Ion Torrent PGM or Proton, the Illumina HiSEQ or MiSEQ, and the Roche/454 next generation sequencing system.

“Detectable label” as used herein refers to a molecule or a compound or a group of molecules or a group of compounds used to identify a nucleic acid or protein of interest. In some embodiments, the detectable label may be detected directly. In other embodiments, the detectable label may be a part of a binding pair, which can then be subsequently detected. Signals from the detectable label may be detected by various means and will depend on the nature of the detectable label. Detectable labels may be isotopes, fluorescent moieties, colored substances, and the like. Examples of means to detect detectable labels include but are not limited to spectroscopic, photochemical, biochemical, immunochemical, electromagnetic, radiochemical, or chemical means, such as fluorescence, chemifluorescence, or chemiluminescence, or any other appropriate means.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, an “expression control sequence” refers to polynucleotide sequences which are necessary to affect the expression of coding sequences to which they are operably linked. Expression control sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to encompass, at a minimum, any component whose presence is essential for expression, and can also encompass an additional component whose presence is advantageous, for example, leader sequences.

“Gene” as used herein refers to a DNA sequence that comprises regulatory and coding sequences necessary for the production of an RNA, which may have a non-coding function (e.g., a ribosomal or transfer RNA) or which may include a polypeptide or a polypeptide precursor. The RNA or polypeptide may be encoded by a full length coding sequence or by any portion of the coding sequence so long as the desired activity or function is retained. Although a sequence of the nucleic acids may be shown in the form of DNA, a person of ordinary skill in the art recognizes that the corresponding RNA sequence will have a similar sequence with the thymine being replaced by uracil, i.e., “T” is replaced with “U.”

As used herein, the term “genome” refers to the whole hereditary information of an organism that is encoded in the DNA (or RNA for certain viral species) including both coding and non-coding sequences. In various embodiments, the term may include the chromosomal DNA of an organism and/or DNA that is contained in an organelle such as, for example, the mitochondria or chloroplasts and/or extrachromosomal plasmid and/or artificial chromosome.

The term “guide sequence” refers to the portion of a crRNA or guide RNA (gRNA) that is responsible for hybridizing with the target DNA.

As used herein, a “heterologous nucleic acid sequence” is any nucleic acid sequence placed at a location where it does not normally occur. A heterologous nucleic acid sequence may comprise a sequence that does not naturally occur in a cell, or it may comprise only sequences naturally found in the cell, but placed at a non-normally occurring location in the cell. In some embodiments, the heterologous nucleic acid sequence is not an endogenous sequence. In certain embodiments, the heterologous nucleic acid sequence is an endogenous sequence that is derived from a different cell. In other embodiments, the heterologous nucleic acid sequence is a sequence that occurs naturally in a cell but is then relocated to another site where it does not naturally occur, rendering it a heterologous sequence at that new site.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. A polynucleotide or polynucleotide region (or a polypeptide or polypeptide region) has a certain percentage (for example, at least 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art. In some embodiments, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+SwissProtein+SPupdate+PIR. Details of these programs can be found at the National Center for Biotechnology Information. Biologically equivalent polynucleotides are those having the specified percent homology and encoding a polypeptide having the same or similar biological activity. Two sequences are deemed “unrelated” or “non-homologous” if they share less than 40% identity, or less than 25% identity, with each other.

As used herein, the phrase “homologous recombination” refers to the process in which nucleic acid molecules with similar nucleotide sequences associate and exchange nucleotide strands. A nucleotide sequence of a first nucleic acid molecule that is effective for engaging in homologous recombination at a predefined position of a second nucleic acid molecule can therefore have a nucleotide sequence that facilitates the exchange of nucleotide strands between the first nucleic acid molecule and a defined position of the second nucleic acid molecule. Thus, the first nucleic acid can generally have a nucleotide sequence that is sufficiently complementary to a portion of the second nucleic acid molecule to promote nucleotide base pairing. Homologous recombination requires homologous sequences in the two recombining partner nucleic acids but does not require any specific sequences. Homologous recombination can be used to introduce a heterologous nucleic acid and/or mutations into the host genome. Such systems typically rely on sequence flanking the heterologous nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

The term “hybridize” as used herein refers to a process where two substantially complementary nucleic acid strands (at least about 65% complementary over a stretch of at least 14 to 25 nucleotides, at least about 75%, or at least about 90% complementary) anneal to each other under appropriately stringent conditions to form a duplex or heteroduplex through formation of hydrogen bonds between complementary base pairs. Hybridizations are typically and preferably conducted with probe-length nucleic acid molecules, preferably 15-100 nucleotides in length, more preferably 18-50 nucleotides in length. Nucleic acid hybridization techniques are well known in the art. See, e.g., Sambrook, et al., 1989, Molecular Cloning: A Laboratory Manual, Second Edition, Cold Spring Harbor Press, Plainview, N.Y. Hybridization and the strength of hybridization (i.e., the strength of the association between the nucleic acids) is influenced by such factors as the degree of complementarity between the nucleic acids, stringency of the conditions involved, and the thermal melting point (Tₘ) of the formed hybrid. Those skilled in the art understand how to estimate and adjust the stringency of hybridization conditions such that sequences having at least a desired level of complementarity will stably hybridize, while those having lower complementarity will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology, John Wiley & Sons, Secaucus, N.J. In some embodiments, specific hybridization occurs under stringent hybridization conditions. An oligonucleotide or polynucleotide (e.g., a probe or a primer) that is specific for a target nucleic acid will “hybridize” to the target nucleic acid under suitable conditions.

As used herein, the terms “individual”, “patient”, or “subject” are used interchangeably and refer to an individual organism, a vertebrate, a mammal, or a human. In a preferred embodiment, the individual, patient or subject is a human.

As used herein, “microbiome” refers to the collective genetic content of the communities of microbes that live in and on the human body, both sustainably and transiently, including eukaryotes, fungi, archaea, bacteria, and viruses (including bacterial viruses (i.e., phage)), wherein “genetic content” includes genomic DNA, RNA such as micro RNA and ribosomal RNA, the epigenome, plasmids, and all other types of genetic information. As used herein, the term “gut microbiome” refers to the collective genetic content of the communities of microbes present in the gastrointestinal tract (GIT).

As used herein, “microbiota” refers to the collective microbes that live in and on the human body, both sustainably and transiently, including eukaryotes, fungi, archaea, bacteria, and viruses (including bacterial viruses (i.e., phage)). “Gut microbiota” as used herein refers to the totality of the microbes present in the GIT, including eukaryotes, fungi, archaea, bacteria, and viruses (including bacterial viruses (i.e., phage)).

“Next-generation sequencing or NGS” as used herein, refers to any sequencing method that determines the nucleotide sequence of either individual nucleic acid molecules (e.g., in single molecule sequencing) or clonally expanded proxies for individual nucleic acid molecules in a high throughput parallel fashion (e.g., greater than 10³, 10⁴, 10⁵ or more molecules are sequenced simultaneously). In one embodiment, the relative abundance of the nucleic acid species in the library can be estimated by counting the relative number of occurrences of their cognate sequences in the data generated by the sequencing experiment. Next generation sequencing methods are known in the art, and are described, e.g., in Metzker, M. Nature Biotechnology Reviews 11:31-46 (2010).

As used herein, “oligonucleotide” refers to a molecule that has a sequence of nucleic acid bases on a backbone comprised mainly of identical monomer units at defined intervals. The bases are arranged on the backbone in such a way that they can bind with a nucleic acid having a sequence of bases that are complementary to the bases of the oligonucleotide. The most common oligonucleotides have a backbone of sugar phosphate units. A distinction may be made between oligodeoxyribonucleotides that do not have a hydroxyl group at the 2′ position and oligoribonucleotides that have a hydroxyl group at the 2′ position. Oligonucleotides may also include derivatives, in which the hydrogen of the hydroxyl group is replaced with organic groups, e.g., an allyl group. Oligonucleotides of the method which function as primers or probes are generally at least about 10-15 nucleotides long and more preferably at least about 15 to 25 nucleotides long, although shorter or longer oligonucleotides may be used in the method. The exact size will depend on many factors, which in turn depend on the ultimate function or use of the oligonucleotide. The oligonucleotide may be generated in any manner, including, for example, chemical synthesis, DNA replication, restriction endonuclease digestion of plasmids or phage DNA, reverse transcription, PCR, or a combination thereof. The oligonucleotide may be modified e.g., by addition of a methyl group, a biotin or digoxigenin moiety, a fluorescent tag or by using radioactive nucleotides.

As used herein, “operably linked” means that expression control sequences are positioned relative to a nucleic acid of interest to initiate, regulate or otherwise control transcription of the nucleic acid of interest. In some embodiments, transcription of a polynucleotide operably linked to an expression control element (e.g., a promoter) is controlled, regulated, or influenced by the expression control element.

As used herein, the term “polynucleotide” or “nucleic acid” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. Nucleic acid molecules can be naturally occurring, recombinant, or synthetic. The term polynucleotide also includes DNAs or RNAs containing one or more modified bases and DNAs or RNAs with backbones modified for stability or for other reasons. Nucleic acid modifications include, for example, methylation, substitution of one or more of the naturally occurring nucleotides with a nucleotide analog, internucleotide modifications such as uncharged linkages (e.g., methyl phosphonates, phosphotriesters, phosphoamidates, carbamates, and the like), charged linkages (e.g., phosphorothioates, phosphorodithioates, and the like), pendent moieties (e.g., polypeptides), intercalators (e.g., acridine, psoralen, and the like), chelators, alkylators, and modified linkages (e.g., alpha anomeric nucleic acids, and the like).

The term “nucleotide sequence,” in reference to a nucleic acid, refers to a contiguous series of nucleotides that are joined by covalent linkages, such as phosphorus linkages (e.g., phosphodiester, alkyl and aryl-phosphonate, phosphorothioate, phosphotriester bonds), and/or non-phosphorus linkages (e.g., peptide and/or sulfamate bonds).

The terms “nucleotide” and “nucleotide monomer” refer to naturally occurring ribonucleotide or deoxyribonucleotide monomers, as well as non-naturally occurring derivatives and analogs thereof. Accordingly, nucleotides can include, for example, nucleotides comprising naturally occurring bases (e.g., adenosine, thymidine, guanosine, cytidine, uridine, inosine, deoxyadenosine, deoxythymidine, deoxyguanosine, or deoxycytidine) and nucleotides comprising modified bases (e.g., 2-aminoadenosine, 2-thiothymidine, pyrrolo-pyrimidine, 3-methyl adenosine, C5-propynylcytidine, C5-propynyluridine, C5-bromouridine, C5-fluorouridine, C5-iodouridine, C5-methyleytidine, 7-deazaadenosine, 7-deazaguanosine, 8-oxoadenosine, 8-oxoguanosine, O(6)-methylguanine, 2-thiocytidine).

A “protospacer sequence” refers to the target double stranded DNA and specifically to the portion of the target DNA (e.g., target region in the genome (e.g., the genome of the target bacterium)) that is fully or substantially complementary (and hybridizes) to a guide sequence of a CRISPR RNA (crRNA). In the case of Type I and II CRISPR-Cas systems, the protospacer sequence is directly followed by a PAM.

The term “protospacer adjacent motif” (or PAM) as used herein, refers to a 2-6 base pair DNA sequence that flanks the DNA region targeted for cleavage by the CRISPR system, such as CRISPR-Cas9. The PAM is required for a Cas nuclease to cut and is generally found 3-4 nucleotides downstream from the cut site. The PAM specificity may be a function of the DNA-binding specificity of the Cas nuclease protein.

As used herein, the term “primer” refers to an oligonucleotide, which is capable of acting as a point of initiation of nucleic acid sequence synthesis when placed under conditions in which synthesis of a primer extension product which is complementary to a target nucleic acid strand is induced, i.e., in the presence of different nucleotide triphosphates and a polymerase in an appropriate buffer (“buffer” includes pH, ionic strength, cofactors etc.) and at a suitable temperature. One or more of the nucleotides of the primer can be modified for instance by addition of a methyl group, a biotin or digoxigenin moiety, a fluorescent tag or by using radioactive nucleotides. A primer sequence need not reflect the exact sequence of the template. For example, a non-complementary nucleotide fragment may be attached to the 5′ end of the primer, with the remainder of the primer sequence being substantially complementary to the strand. The term primer as used herein includes all forms of primers that may be synthesized including peptide nucleic acid primers, locked nucleic acid primers, phosphorothioate modified primers, labeled primers, and the like. The term “forward primer” as used herein means a primer that anneals to the anti-sense strand of dsDNA. A “reverse primer” anneals to the sense-strand of dsDNA.

As used herein, “primer pair” refers to a forward and reverse primer pair (i.e., a left and right primer pair) that can be used together to amplify a given region of a nucleic acid of interest.

“Probe” as used herein refers to nucleic acid that interacts with a target nucleic acid via hybridization. A probe may be fully complementary to a target nucleic acid sequence or partially complementary. The level of complementarity will depend on many factors based, in general, on the function of the probe. A probe or probes can be used, for example to detect the presence or absence of a mutation in a nucleic acid sequence by virtue of the sequence characteristics of the target. Probes can be labeled or unlabeled, or modified in any of a number of ways well known in the art. A probe may specifically hybridize to a target nucleic acid. Probes may be DNA, RNA or a RNA/DNA hybrid. Probes may be oligonucleotides, artificial chromosomes, fragmented artificial chromosome, genomic nucleic acid, fragmented genomic nucleic acid, RNA, recombinant nucleic acid, fragmented recombinant nucleic acid, peptide nucleic acid (PNA), locked nucleic acid, oligomer of cyclic heterocycles, or conjugates of nucleic acid. Probes may comprise modified nucleobases, modified sugar moieties, and modified internucleotide linkages. A probe may be used to detect the presence or absence of a target nucleic acid. Probes are typically at least about 10, 15, 20, 25, 30, 35, 40, 50, 60, 75, 100 nucleotides or more in length.

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors.

As used herein, the term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the material is derived from a cell so modified. Thus, for example, recombinant cells express genes that are not found within the native (non-recombinant) form of the cell or express native genes that are otherwise abnormally expressed, under expressed or not expressed at all.

As used herein, an endogenous nucleic acid sequence in the cell of an organism (or the encoded protein product of that sequence) is deemed “recombinant” herein if a heterologous sequence is placed adjacent to the endogenous nucleic acid sequence, such that the expression of this endogenous nucleic acid sequence is altered. In this context, a heterologous sequence is a sequence that is not naturally adjacent to the endogenous nucleic acid sequence, whether or not the heterologous sequence is itself endogenous to the organism (originating from the same organism or progeny thereof) or exogenous (originating from a different organism or progeny thereof). By way of example, a promoter sequence can be substituted (e.g., by homologous recombination) for the native promoter of a gene in the cell of an organism, such that this gene has an altered expression pattern. This gene would be “recombinant” because it is separated from at least some of the sequences that naturally flank it. A nucleic acid is also considered “recombinant” if it contains any modifications that do not naturally occur in the corresponding nucleic acid in a cell. For instance, an endogenous coding sequence is considered “recombinant” if it contains an insertion, deletion or a point mutation introduced artificially, e.g., by human intervention. A “recombinant nucleic acid” also includes a nucleic acid integrated into a host cell chromosome at a heterologous site and a nucleic acid construct present as an episome.

As used herein, a “reporter gene” refers to a polynucleotide sequence encoding a gene product (e.g., polypeptide) that can generate, under appropriate conditions, a detectable signal that allows detection of the presence and/or quantity of the gene product. Reporter genes are often used as an indication of whether a certain gene has been introduced into or expressed in the host cell or organism. Examples of commonly used reporters include: antibiotic resistance genes, fluorescent proteins, auxotropic selection modules, β-galactosidase (encoded by the bacterial gene lacZ), luciferase (from lightning bugs), chloramphenicol acetyltransferase (CAT; from bacteria), GUS (β-glucuronidase; commonly used in plants) and green fluorescent protein (GFP; from jelly fish). Reporters or selection moduless can be selectable or screenable.

As used herein, a “sample” refers to a substance that is being assayed for the presence of a mutation in a nucleic acid of interest. Processing methods to release or otherwise make available a nucleic acid for detection are well known in the art and may include steps of nucleic acid manipulation. A biological sample may be a body fluid or a tissue sample. In some cases, a biological sample may consist of or comprise blood, plasma, sera, urine, feces, epidermal sample, vaginal sample, skin sample, cheek swab, sperm, amniotic fluid, cultured cells, bone marrow sample, aspirate and/or chorionic villi, cultured cells, and the like.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

The term “stringent hybridization conditions” as used herein refers to hybridization conditions at least as stringent as the following: hybridization in 50% formamide, 5×SSC, 50 mM NaH₂PO₄, pH 6.8, 0.5% SDS, 0.1 mg/mL sonicated salmon sperm DNA, and 5× Denhart’s solution at 42° C. overnight; washing with 2× SSC, 0.1% SDS at 45° C.; and washing with 0.2× SSC, 0.1% SDS at 45° C. In another example, stringent hybridization conditions should not allow for hybridization of two nucleic acids which differ over a stretch of 20 contiguous nucleotides by more than two bases.

As used herein, the term “therapeutic agent” is intended to mean a compound that, when present in an effective amount, produces a desired therapeutic effect on a subject in need thereof.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

As used herein, a “vector” refers to a nucleic acid molecule capable of transporting another nucleic acid to which it has been linked. One type of vector is a “plasmid,” which generally refers to a circular double stranded DNA loop into which additional DNA segments may be ligated, but also includes linear double- stranded molecules such as those resulting from amplification by the polymerase chain reaction (PCR) or from treatment of a circular plasmid with a restriction enzyme. Other vectors include cosmids, bacterial artificial chromosomes (BAC) and yeast artificial chromosomes (YAC). Another type of vector is a viral vector, wherein additional DNA segments may be ligated into the viral genome. Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain vectors are capable of directing the expression of genes to which they are operatively linked. Such vectors are referred to herein as “recombinant expression vectors” (or simply “expression vectors”).

### Candida-Compatible Nucleic Acids Encoding CRISPR/Cas9 System Components

The expression vector system of the present technology comprises a Candida-compatible Cas9 nuclease and a synthetic guide RNA (sgRNA) that directs Cas9 to cleave regions in the genome that hybridize to the 20 bp guide (or protospacer) from the sgRNA when it is followed by the sequence NGG (the protospacer-adjacent motif, or “PAM”). This system has been successfully imported to diverse kingdoms ranging from fungi to plants and animals (reviewed in Doudna and Charpentier, Science 346:1258096 (2014); Terns and Terns, Trends Genet 30:111-118 (2014)). However, most of these systems do not pose the unique set of constraints found in Candida.

The expression vector system described herein is based, in part, on the identification of a codon-optimized sequence for expressing Cas9 protein in various species of Candida and other species of yeast (e.g., CTG clade species of yeast). Thus, the present CRISPR/Cas9 system is compatible for use in various yeasts, including Candida.

The nucleic acids described herein relate, in part, to a “Duet” system, and a “Solo” system for performing CRISPR in yeast (e.g., Candida), which is described in US20170166928, the contents of which are herein incorporated in their entirety. The Duet system, uses the sequential integration of two plasmids: the first comprising CaCas9 nucleotide sequence (the “Duet CaCas9 system plasmid” e.g., pV1025) and the second comprising a coding sequence for a synthetic guide RNA (sgRNA) that targets a gene of interest (the “Duet sgRNA system plasmid”, e.g., pV1090). The Duet sgRNA system plasmid allows a user to insert any suitable sgRNA coding sequence designed for a target sequence of interest. In general, the second plasmid for expression of the sgRNA against a target gene is cotransformed with a mutagenic double-stranded oligonucleotide (a “repair template”, as described herein), which is complementary to a target gene and may contain a desired modification, e.g., a mutation to the PAM sequence and a premature UAA stop codon.

The “Solo” system, consolidates the CaCas9 nucleotide sequence and the sgRNA coding sequence into a single plasmid construct (the “Solo CaCas9/sgRNA system plasmid”) that can be integrated at a desired locus. Like the Duet system, a mutagenic double-stranded oligonucleotide can be cotransformed with the Solo system. Similar to the Duet sgRNA system plasmid, the Solo system allows the insertion of any suitable sgRNA coding sequence designed for a target sequence of interest

Accordingly, in certain aspects, the expression vector system includes a nucleic acid comprising a Candida-compatible clustered regularly interspaced short palindromic repeat (CRISPR)-associated nuclease 9 (Cas9) (CaCas9) nucleotide sequence. As used herein, a “Candida-compatible Cas9 nucleotide sequence” or “CaCas9 nucleotide sequence” refers to a nucleotide sequence encoding a bacterial Cas9 protein (e.g., a Cas9 nuclease from any of a variety of prokaryotes, such as, for example, Streptococcus pyogenes, Staphylococcus aureus, Neisseria meningitides, Streptococcus thermophilus, and Treponema denticola), wherein the bacterial Cas9 nucleotide sequence has been optimized (e.g., codon optimized) for expression of the bacterial Cas9 protein in Candida. As those of skill in the art would appreciate in light of the present disclosure, other endonucleases known in the art can also be used in the present technology. See, e.g., Zetsche et al., Cell 163(3):759-71, 2015; Kleinstiver et al., Nature 523(7561):481-85, 2015-each incorporated herein by reference in its entirety).

Many species of Candida belong to the fungal CTG clade corresponding to a group of ascomycetous yeasts displaying a particular genetic code, such that the universal CUG codon for leucine is predominantly translated as serine and rarely as leucine (Papon, et al., Trends in Biotechnology 32(4):167-68, 2014). Thus, a CaCas9 nucleotide sequence can be prepared, for example, by encoding one or more (e.g., all), of the leucine residues in a Cas9 protein sequence (e.g., SEQ ID NO: 25) with a codon other than CTG or CUG, e.g., CTC, TTG, CTT, CTA, and TTA.

Nuclease-inactive Cas9 Protein Sequence (e.g., SEQ ID NO: 25)

MDKKYSIGLD IGTNSVGWAV ITDEYKVPSK KFKVLGNTDR HSIKKNLIGA
LLFDSGETAE ATRLKRTARR RYTRRKNRIC YLQEIFSNEM AKVDDSFFHR
LEESFLVEED KKHERHPIFG NIVDEVAYHE KYPTIYHLRK KLVDSTDKAD
LRLIYLALAH MIKFRGHFLI EGDLNPDNSD VDKLFIQLVQ TYNQLFEENP
INASGVDAKA ILSARLSKSR RLENLIAQLP GEKKNGLFGN LIALSLGLTP
NFKSNFDLAE DAKLQLSKDT YDDDLDNLLA QIGDQYADLF LAAKNLSDAI
LLSDILRVNT EITKAPLSAS MIKRYDEHHQ DLTLLKALVR QQLPEKYKEI
FFDQSKNGYA GYIDGGASQE EFYKFIKPIL EKMDGTEELL VKLNREDLLR
KQRTFDNGSI PHQIHLGELH AILRRQEDFY PFLKDNREKI EKILTFRIPY
YVGPLARGNS RFAWMTRKSE ETITPWNFEE VVDKGASAQS FIERMTNFDK
NLPNEKVLPK HSLLYEYFTV YNELTKVKYV TEGMRKPAFL SGEQKKAIVD
LLFKTNRKVT VKQLKEDYFK KIECFDSVEI SGVEDRFNAS LGTYHDLLKI
IKDKDFLDNE ENEDILEDIV LTLTLFEDRE MIEERLKTYA HLFDDKVMKQ
LKRRRYTGWG RLSRKLINGI RDKQSGKTIL DFLKSDGFAN RNFMQLIHDD
SLTFKEDIQK AQVSGQGDSL HEHIANLAGS PAIKKGILQT VKVVDELVKV
MGRHKPENIV IEMARENQTT QKGQKNSRER MKRIEEGIKE LGSQILKEHP
VENTQLQNEK LYLYYLQNGR DMYVDQELDI NRLSDYDVDH IVPQSFLKDD
SIDNKVLTRS DKNRGKSDNV PSEEVVKKMK NYWRQLLNAK LITQRKFDNL
TKAERGGLSE LDKAGFIKRQ LVETRQITKH VAQILDSRMN TKYDENDKLI
REVKVITLKS KLVSDFRKDF QFYKVREINN YHHAHDAYLN AVVGTALIKK
YPKLESEFVY GDYKVYDVRK MIAKSEQEIG KATAKYFFYS NIMNFFKTEI
TLANGEIRKR PLIETNGETG EIVWDKGRDF ATVRKVLSMP QVNIVKKTEV
QTGGFSKESI LPKRNSDKLI ARKKDWDPKK YGGFDSPTVA YSVLVVAKVE
KGKSKKLKSV KELLGITIME RSSFEKNPID FLEAKGYKEV KKDLIIKLPK
YSLFELENGR KRMLASAGEL QKGNELALPS KYVNFLYLAS HYEKLKGSPE
DNEQKQLFVE QHKHYLDEII EQISEFSKRV ILADANLDKYV LSAYNKHRDK
PIREQAENII HLFTLTNLGA PAAFKYFDTT IDRKRYTSTK EVLDATLIHQ
SITGLYETRI DLSQLGGDEG A

However, serine residues in a Cas9 protein sequence can be encoded by a CTG or CUG codon, as well as any other serine codon. In further aspects, a leucine residue in Cas9 can be encoded by CTG or CUG if a substitution of that leucine residue for serine does not substantially alter the function of Cas9. In various aspects, while “Candida-compatible” refers to a coding sequence optimized for expression in Candida, those of skill in the art will appreciate, in light of the present disclosure, that the nucleotide sequences disclosed herein may be used and expressed in a variety of yeast species (e.g., C. albicans), as described herein. Codon optimization in yeast is described, for example, in U.S. Pat. Application Publication No. 20120309073, the contents of which are incorporated herein by reference.

In some aspects, the nucleic acid comprising a Candida-compatible clustered regularly interspaced short palindromic repeat (CRISPR)-associated nuclease 9 (Cas9) nucleotide sequence (CaCas9) that encodes a protein having at least about 40%, 50%, 60%, 70%, 80%, 85%, 90%, 93%, 94%, 95%, 96%, 97%, 98%, or 99% sequence identity to SEQ ID NO: 25, or a fragment thereof, wherein each leucine in the protein is encoded by a codon other than CTG, e.g., CTC, TTG, CTT, CTA, and TTA. In certain aspects, the nucleic acid comprises a CaCas9 nucleotide sequence that encodes SEQ ID NO: 25. In other aspects, the nucleic acid comprises a CaCas9 nucleotide sequence that encodes SEQ ID NO: 26.

Nuclease-inactive Cas9 Protein D10A, H840A Mutant Sequence

MDKKYSIGLA IGTNSVGWAV ITDEYKVPSK KFKVLGNTDR HSIKKNLIGA
LLFDSGETAE ATRLKRTARR RYTRRKNRIC YLQEIFSNEM AKVDDSFFHR
LEESFLVEED KKHERHPIFG NIVDEVAYHE KYPTIYHLRK KLVDSTDKAD
LRLIYLALAH MIKFRGHFLI EGDLNPDNSD VDKLFIQLVQ TYNQLFEENP
INASGVDAKA ILSARLSKSR RLENLIAQLP GEKKNGLFGN LIALSLGLTP
NFKSNFDLAE DAKLQLSKDT YDDDLDNLLA QIGDQYADLF LAAKNLSDAI
LLSDILRVNT EITKAPLSAS MIKRYDEHHQ DLTLLKALVR QQLPEKYKEI
FFDQSKNGYA GYIDGGASQE EFYKFIKPIL EKMDGTEELL VKLNREDLLR
KQRTFDNGSI PHQIHLGELH AILRRQEDFY PFLKDNREKI EKILTFRIPY
YVGPLARGNS RFAWMTRKSE ETITPWNFEE VVDKGASAQS FIERMTNFDK
NLPNEKVLPK HSLLYEYFTV YNELTKVKYV TEGMRKPAFL SGEQKKAIVD
LLFKTNRKVT VKQLKEDYFK KIECFDSVEI SGVEDRFNAS LGTTEDLLKI
IKDKDFLDNE ENEDILEDIV LTLTLFEDRE MIEERLKTYA HLFDDKVMKQ
LKRRRYTGWG RLSRKLINGI RDKQSGKTIL DFLKSDGFAN RNFMQLIHDD
SLTFKEDIQK AQVSGQGDSL HEHIANLAGS PAIKKGILQT VKVVDELVKV
MGRHKPENIV IEMARENQTT QKGQKNSRER MKRIEEGIKE LGSQILKEHP
VENTQLQNEK LYLYYLOQNGR DMYVDQELDI NRLSDYDVDA IVPQSFLKDD
SIDNKVLTRS DKNRGKSDNV PSEEVVKKMK NYWRQLLNAK LITQRKFDNL
TKAERGGLSE LDKAGFIKRQ LYETRQITKH VAQILDSRMN TKYDENDKLI
REVKVITLKS KLVSDFRKDF QFYKVREINN YHHAHDAYLN AVVGTALIKK
YPKLESEFVY GDYKVYDVRK MIAKSEQEIG KATAKYFFYS NIMNFFKTEI
TLANGEIRKR PLIETNGETG EIVWDKGRDF ATVRKVLSMP QVNIVKKTEV
QTGGFSKESI LPKRNSDKLI ARKKDWDPKK YGGFDSPTVA YSVLVVAKVE
KGKSKKLKSY KELLGITIME RSSFEKNPID FLEAKGYKEV KKDLIIKLPK
YSLFELENGR KRMLASAGEL QKGNELALPS KYVNFLYLAS HYEKLKGSPE
DNEQKQLEFVE QHKHYLDEII EQISEFSKRV ILADANLDKV LSAYNKHRDK
PIREQAENII HLFTLTNLGA PAAFKYFDTT IDRKRYTSTK EVLDATLIHQ
SITGLYETRI DLSQLGGDEG A (SEQ ID NO: 26)

As used herein, a “fragment” of a Cas9 protein includes any nuclease-active or nuclease-inactive portion of a Cas9 protein. For example, the nucleic acid may encode one or more fragments of Cas9 that retains nuclease activity. In a particular example, Cas9 may be expressed as two separate fragments (e.g., a nuclease lobe and an alpha-helical lobe) which form a functional, active complex in the presence of an sgRNA (see, e.g., Wright, et al., PNAS, 112 (10:2984-89), 2015). In other aspects, the nucleic acid may encode a nuclease-inactive fragment of Cas9 which may, for example, be fused to one or more other genes (e.g., a transcriptional repressor or activator).

In certain embodiments, the nucleic acid comprising a Candida-compatible clustered regularly interspaced short palindromic repeat (CRISPR)-associated nuclease 9 (CaCas9) nucleotide sequence is a codon-optimized sequence of SEQ ID NO: 27.

CaCas9 encoding nucleotide sequence

ATGGATAAGA AATACTCAAT AGGCTTAGAT ATCGGCACAA ATAGCGTCGG
ATGGGCGGTG ATCACTGATG AATATAAGGT TCCGTCTAAA AAGTTCAAGG
TTCTGGGAAA TACAGACCGC CACAGTATCA AAAAAAATCT TATAGGGGCT
CTTTTATTTG ACAGTGGAGA GACAGCGGAA GCGACTCGTC TCAAACGGAC
AGCTCGTAGA AGGTATACAC GTCGGAAGAA TCGTATTTGT TATCTACAGG
AGATTTTTTC AAATGAGATG GCGAAAGTAG ATGATAGTTT CTTTCATCGA
CTTGAAGAGT CTTTTTTGGT GGAAGAAGAC AAGAAGCATG AACGTCATCC
TATTTTTGGA AATATAGTAG ATGAAGTTGC TTATCATGAG AAATATCCAA
CTATCTATCA TCTGCGAAAA AAATTGGTAG ATTCTACTGA TAAAGCGGAT
TTGCGCTTAA TCTATTTGGC CTTAGCGCAT ATGATTAAGT TTCGTGGTCA
TTTTTTGATT GAGGGAGATT TAAATCCTGA TAATAGTGAT GTGGACAAAC
TATTTATCCA GTTGGTACAA ACCTACAATC AATTATTTGA AGAAAACCCT
ATTAACGCAA GTGGAGTAGA TGCTAAGCGA TTCTTTCTGC ACGATTGAGT
AAATCAAGAC GATTAGAAAA TCTCATTGCT CAGCTCCCCG GTGAGAAGAA
AAATGGCTTA TTTGGGAATC TCATTGCTTT GTCATTGGGT TTGACCCCTA
ATTTTAAATC AAATTTTGAT TTGGCAGAAG ATGCTAAATT ACAGCTTTCA
AAAGATACTT ACGATGATGA TTTAGATAAT TTATTGGCGC AAATTGGAGA
TCAATATGCT GATTTGTTTT TGGCAGCTAA GAATTTATCA GATGCTATTT
TACTTTCAGA TATCCTAAGA GTAAATACTG AAATAACTAA GGCTCCCCTA
TCAGCTTCAA TGATTAAACG CTACGATGAA CATCATCAAG ACTTGACTCT
TTTAAAAGCT TTAGTTCGAC AACAACTTCC AGAAAAGTAT AAAGAAATCT
TTTTTGATCA ATCAAAAAAC GGATATGCAG GTTATATTGA TGGGGGAGCT
AGCCAAGAAG AATTTTATAA ATTTATCAAA CCAATTTTAG AAAAAATGGA
TGGTACTGAG GAATTATTGG TGAAACTAAA TCGTGAAGAT TTGCTGCGCA
AGCAACGGAC CTTTGACAAC GGCTCTATTC CCCATCAAAT TCACTTGGGT
GAGCTGCATG CTATTTTGAG AAGACAAGAA GACTTTTATC CATTTTTAAA
AGACAATCGT GAGAAGATTG AAAAAATCTT GACTTTTCGA ATTCCTTATT
ATGTTGGTCC ATTGGCGCGT GCCAATAGTC GTTTTCCATG GATGACTCGG
AAGTCTGAAG AAACAATTAC CCCATGGAAT TTTGAAGAAG TTGTCGATAA
AGGTGCTTCA GCTCAATCAT TTATTGAACG CATGACAAAC TTTGATAAAA
ATCTTCCAAA TGAAAAAGTA CTACCAAAAC ATAGTTTGCT TTATGAGTAT
TTTACGGTTT ATAACGAATT GACAAAGGTC AAATATGTTA CTGAAGGAAT
GCGAAAACCA GCATTTCTTT CAGGTGAACA GAAGAAAGCC ATTGTTGATT
TACTCTTCAA AACAAATCGA AAAGTAACCG TTAAGCAATT AAAAGAAGAT
TATTTCAAAA AAATAGAATG TTTTGATAGT GTTGAAATTT CAGGAGTTGA
AGATAGATTT AATGCTTCAT TAGGTACCTA CCATGATTTG CTAAAAATTA
TTAAAGATAA AGATTTTTTG GATAATGAAG AAAATGAAGA TATCTTAGAG
GATATTGTTT TAACATTGAC CTTATTTGAA GATAGGGAGA TGATTGAGGA
AAGACTTAAA ACATATGCTC ACCTCTTTGA TGATAAGGTG ATGAAACAGC
TTAAACGTCG CCGTTATACT GGTTGGGGAC GTTTGTCTCG AAAATTGATT
AATGGTATTA GGGATAAGCA ATCTGGCAAA ACAATATTAG ATTTTTTGAA
ATCAGATGGT TTTGCCAATC GCAATTTTAT GCAGCTGATC CATGATGATA
GTTTGACATT TAAAGAAGAC ATTCAAAAAG CACAAGTGTC TGGACAAGGC
GATAGTTTAC ATGAACATAT TGCAAATTTA GCTGGTAGCC CTGCTATTAA
AAAAGGTATT TTACAGACTG TAAAAGTTGT TGATGAATTG GTCAAAGTAA
TGGGGCGGCA TAAGCCAGAA AATATCGTTA TTGAAATGGC ACGTGAAAAT
CAGACAACTC AAAAGGGCCA GAAAAATTCG CGAGAGCGTA TGAAACGAAT
CGAAGAAGGT ATCAAAGAAT TAGGAAGTCA GATTCTTAAA GAGCATCCTG
TTGAAAATAC TCAATTGCAA AATGAAAAGC TCTATCTCTA TTATCTCCAA
AATGGAAGAG ACATGTATGT GGACCAAGAA TTAGATATTA ATCGTTTAAG
TGATTATGAT GTCGATCACA TTGTTCCACA AAGTTTCCTT AAAGACGATT
CAATAGACAA TAAGGTCTTA ACGCGTTCTG ATAAAAATCG TGGTAAATCG
GATAACGTTC CAAGTGAAGA AGTAGTCAAA AAGATGAAAA ACTATTGGAG
ACAACTTCTA AACGCCAAGT TAATCACTCA ACGTAAGTTT GATAATTTAA
CGAAAGCTGA ACGTGGAGGT TTGAGTGAAC TTGATAAAGC TGGTTTTATC
AAACGCCAAT TGGTTGAAAC TCCCCAAATC ACTAAGCATG TGGCACAAAT
TTTGGATAGT CGCATGAATA CTAAATACGA TGAAAATGAT AAACTTATTC
GAGAGGTTAA AGTGATTACC TTAAAATCTA AATTAGITTC TGACTTCCGA
AAAGATTTCC AATTCTATAA AGTACGTGAG ATTAACAATT ACCATCATGC
CCATGATGCG TATCTAAATG CCGTCGTTGG AACTGCTTTG ATTAAGAAAT
ATCCAAAACT TGAATCGGAG TTTGTCTATG GTGATTATAA AGTTTATGAT
GTTCGTAAAA TGATTGCTAA GTCTGAGCAA GAAATAGGCA AAGCAACCGC
AAAATATTTC TTTTACTCTA ATATCATGAA CTTCTTCAAA ACAGAAATTA
CACTTGCAAA TGGAGAGATT CGCAAACCCC CTCTAATCGA AACTAATGGG
GAAACTGGAG AAATTGTCTG GGATAAAGGG CGAGATTTTG CCACAGTGCG
CAAAGTATTG TCCATGCCCC AAGTCAATAT TGTCAAGAAA ACAGAAGTAC
AGACAGGCGG ATTCTCCAAG GAGTCAATTT TACCAAAAAG AAATTCGGAC
AAGCTTATTG CTCGTAAAAA AGACTGGGAT CCAAAAAAAT ATGGTGGTTT
TGATAGTCCA ACGGTAGCTT ATTCAGTCCT AGTGGTTGCT AAGGTCIGAAA
AAGGGAAATC GAAGAAGTTA AAATCCGTTA AAGAGTTACT AGGGATCACA
ATTATGGAAA GAAGTTCCTT TGAAAAAAAT CCGATTGACT TTTTAGAAGC
TAAAGGATAT AAGGAAGTTA AAAAAGACTT AATCATTAAA CTACCTAAAT
ATAGTCTTIT TGAGTTAGAA AACGGTCGTA AACGGATGCT GGCTAGTGCC
GGAGAATTAC AAAAAGGAAA TGAGCTGGCT CTGCCAAGCA AATATGTGAA
TTTTTTATAT TTAGCTAGTC ATTATGAAAA GTTGAAGGGT AGTCCAGAAG
ATAACGAACA AAAACAATTG TTTGTGGAGC AGCATAAGCA TTATTTAGAT
GAGATTATTG AGCAAATCAG TGAATTTTCT AAGCGTGTTA TTTTAGCAGA
TGCCAATTTA GATAAAGTTC TTAGTGCATA TAACAAACAT AGAGACAAAC
CAATACGTGA ACAAGCAGAA AATATTATTC ATTTATTTAC GTTGACGAAT
CTTGGAGCTC CCGCTGCTTT TAAATATTTT GATACAACAA TTGATCGTAA
ACGATATACG TCTACAAAAG AAGTTTTAGA TGCCACTCTT ATCCATCAAT
CCATCACTGG TCTTTATGAA ACACGCATTG ATTTGAGTCA GCTAGGAGGT
GAC (SEQ ID NO. 27)

In certain aspects, the CaCas9 nucleotide sequence has at least about 50%, 60%, 70%, 75%, 80%, 85%, 90%, 92%, 93%, 94%, 95%, 96%, 97%, 98%, or 99% sequence identity to SEQ ID NO: 28. In a particular aspect, the CaCas9 nucleotide sequence comprises SEQ ID NO: 28.

Streptococcus pyogenes Cas9 nucleotide sequence (codon optimized variant)

ATGGATAAAA AGTATAGTAT TGGTTTAGAT ATTGGTACTA ACTCTGTGGG
TTGGGCAGTT ATCACCGACG AATATAAAGT TCCATCAAAG AAATTTAAGG
TGTTAGGTAA CACTGACAGA CACTCAATAA AAAAGAATCT TATCGGTGCT
CTTTTGTTCG ACTCCGGTGA AACTCCCGAG GCTACACGTT TAAAAAGAAC
AGCAAGAAGA AGATATACCC GTAGAAAAAA TAGAATATGT TATTTACAAG
AAATCTTTTC TAATGAAATG GCTAAAGTTG ATGATTCCTT TTTCCATAGA
TTGGAAGAGT CATTTTTGGT TGAAGAAGAC AAAAAGCATG AGAGACATCC
AATCTTTGGG AATATAGTTG ATGAAGTGGC TTACCATGAA AAATATCCTA
CCATTTATCA TTTAAGAAAG AAATTGGTAG ATTCAACTGA TAAAGCTGAC
CTTAGATTAA TCTATTTAGC ACTTGCCCAT ATGATTAAAT TTAGAGGTCA
TTTTTTGATT GAAGGTGATT TGAACCCAGA TAATTCTGAC GTGGATAAAT
TATTTATTCA ATTAGTCCAA ACCTACAACC AATTATTTGA GGAAAATCCA
ATTAATGCTA GTGGTGTCGA TGCCAAAGCT ATATTATCAG CCAGATTATC
AAAATCTAGA CGTTTGGAAA ATTTGATTGC CCAATTGCCA GGAGAAAAAA
AGAATGGATT ATTTGGAAAC TTGATCGCAT TATCATTGGG TTTGACACCA
AATTTTAAAT CTAATTTTGA TTTAGCTGAA GATGCTAAAT TACAATTATC
AAAAGACACC TATGACGACG ATTTGGACAA TTTACTTCCT CAAATTGGTG
ATCAATATGC AGATTTGTTC TTAGCTGCTA AAAACTTATC TGATGCTATT
TTGTTGTCTG ATATTTTGAG AGTGAACACA GAAATAACCA AAGCTCCATT
ATCAGCATCT ATGATCAAAC GTTATGATGA ACACCATCAG GATTTGACTT
TATTGAAAGC TTTGGTGAGA CAACAATTGC CAGAGAAGTA TAAAGAAATC
TTTTTCGATC AATCTAAAAA CGGGTATGCA GGTTATATTG ATGGGGGTGC
CTCCCAAGAG GAATTTTACA AATTTATAAA ACCTATTTTA GAAAAGATGG
ATGGGACTGA GGAACTITTG GTCAAATTGA ACAGAGAAGA TTTGTTACGT
AAACAGAGAA CTTTTGATAA TGGTAGTATA CCTCACCAAA TTCATITGGG
TGAGTTGCAT GCAATTTTAA GAAGACAAGA AGATTTTTAT CCATTTTTAA
AAGATAATAG AGAAAAAATC GAGAAAATTT TAACCTTTAG AATTCCATAC
TATGTTGGGC CTTTGGCTAG AGGTAATTCA AGATTTGCCT GGATGACACG
TAAATCAGAA GAAACTATTA CCCCTTGGAA TTTTGAAGAG GTTGTTGATA
AAGGAGCATC AGCACAGAGT TTTATTGAAA GAATGACCAA TTTCGATAAA
AACTTACCAA ATGAAAAAGT TTTACCAAAA CATTCCTTGT TATACGAATA
TTTTACTGTT TACAATGAAC TTACAAAGGT TAAATATGTT ACTGAAGGTA
TGCGTAAGCC AGCCTTTTTA TCTGGAGAAC AGAAAAAGGC AATAGTTGAT
TTATTGTTTA AAACAAATAG AAAAGTTACT GTTAAACAAT TAAAAGAAGA
TTACTTTAAG AAAATTGAAT GTTTTGATTC AGTTGAAATC AGTGGTGTTG
AAGACAGATT TAATGCTAGT TTAGGAACTT ACCATGATTT ACTTAAAATT
ATCAAAGATA AAGATTTCTT GGATAACGAA GAAAATGAAG ACATTTTAGA
AGACATTGTT TTAACCTTAA CTTTATTCGA AGATAGAGAG ATGATTGAAG
AACGTTTGAA GACTTATGCA CATTTGTTTG ACGATAAAGT GATGAAACAG
TTGAAAAGAA GACGITATAC TGGATGGGOGT AGATTGTCTC GTAAATTGAT
CAATGGAATT AGAGATAAAC AAAGTGGTAA AACTATCTTG GACTTTTTGA
AATCTGACGG ATTTGCTAAT AGAAATTTCA TGCAATTGAT CCACGACGAT
AGTTTGACAT TTAAAGAAGA CATCCAAAAG GCCCAAGTGA GTGGGCAAGG
TGATTCATTA CATGAACATA TTGCAAATTT AGCCGGATCT CCTGCTATTA
AGAAAGGGAT ATTACAAACT GTTAAAGTTG TGGATGAATT AGTGAAAGTA
ATGGGAAGAC ATAAACCTGA AAACATTGTC ATTGAGATGG CAAGAGAAAA
TCAAACTACA CAAAAAGGAC AGAAAAATAG TAGAGAACGT ATGAAAAGAA
TAGAAGAGGG TATTAAAGAA TTGGGTAGTC AAATATTGAA AGAACACCCA
GTGGAAAATA CCCAGTTGCA AAATGAAAAA TTATATCTTT ACTACCTTCA
AAATGGACGT GATATGTATG TTGATCAGGA ATTAGATATA AATAGACTTT
CAGATTATGA TGTAGATCAT ATAGTTCCAC AATCTTTCTT GAAAGATGAT
TCCATAGACA ATAAAGTATT AACTAGAAGT GATAAAAATA GAGGTAAAAG
TGATAATGTC CCAAGTGAGG AAGTCGTCAA AAAGATGAAA AATTACTGGC
GTCAACTTTT GAATGCTAAA TTAATTACTC AAAGAAAATT TGATAATTTG
ACTAAAGCAG AAAGAGGTGG GCTTTCTGAA TTAGATAAAG CCGGGTTCAT
TAAAAGACAA TTGGTCGAAA CTAGACAAAT TACTAAACAT GTTGCCCAAA
TTTTAGATTC CCGTATGAAC ACTAAGTATG ACGAAAATGA TAAGTTAATA
CGTGAGGTTA AAGTCATTAC TTTAAAATCA AAACTTGTCT CTGATTTCAG
AAAGGATTTC CAATTCTATA AAGTTAGAGA AATTAATAAT TATCATCATG
CTCATGATGC ATATTTGAAT GCTGTAGTTG GAACTGCTTT AATCAAGAAA
TACCCTAAAT TAGAATCTGA ATTTGTATAT GGTGATTACA AAGTCTATGA
TGTTAGAAAG ATGATTGCTA AATCAGAACA AGAAATTGGT AAAGCTACAG
CTAAATACTT CTTTTACTCT AACATTATGA ATTTCTTTAA AACAGAAATT
ACTTTGGCAA ACGGTGAAAT TAGAAAAAGA CCTCTTATTG AAACAAATGG
TGAGACTGGA GAGATAGTTT GGGACAAAGG GCGTGATTTC GCTACTGTTA
GAAAAGTTTT ATCAATGCCA CAAGTTAACA TTGTAAAGAA AACAGAGGTT
CAAACTGGTG GTTTCTCAAA AGAAAGTATT TTGCCTAAAA GAAATAGTGA
TAAATTGATT GCCAGAAAAA AGGATTGGGA TCCAAAGAAA TATGGTGGTT
TCGACTCACC AACCGTAGCC TATTCTGTTT TGGTTGTGGC AAAGGTTGAA
AAGGGTAAAA GTAAAAAGCT TAAATCAGTA AAAGAACTTT TGGGTATTAC
AATAATGGAA AGAAGTTCCT TTGAAAAGAA CCCTATTGAT TTTTTGGAAG
CTAAAGGTTA TAAGGAAGTA AAGAAGGACT TAATAATCAA ATTGCCTAAA
TATTCTTTAT TTGAATTAGA AAATGGGAGA AAAAGAATGT TGGCTTCTGC
TGGAGAATTG CAAAAGGGTA ATGAATTAGC ATTGCCTTCC AAATATGTTA
ACTTCTTGTA TTTAGCTTCA CACTATGAAA AGTTGAAAGG GTCACCAGAA
GATAACGAGC AAAAACAATT ATTTGTTGAA CAACACAAAC ACTACTTAGA
TGAGATTATA GAACAAATTA GTGAATTCAG TAAAAGAGTG ATATTAGCTG
ATGCAAATTT AGATAAAGTT TTGTCAGCCT ATAACAAACA TAGAGATAAG
CCAATTAGAG AACAAGCAGA AAACATTATT CACTTATTTA CCCTTACCAA
TTTAGGAGCA CCTGCTGCTT TCAAGTATTT TGATACAACA ATTGATCGTA
AAAGATATAC CTCAACAAAA GAAGTCTTAG ACGCCACCTT AATTCATCAA
TCAATCACTG GATTGTATGA GACAAGAATT GATTTGTCTC AATTGGGTGG
TGATGAAGGG GCT (SEQ ID NO: 28)

As used herein, “wild-type” in the context of a Cas9 coding sequence or protein refers to the canonical bacterial nucleotide or amino acid sequence as found in nature (e.g., as occurs in the bacterium Streptococcus pyogenes). A particular example of a wild-type Cas9 coding sequence is SEQ ID NO: 27. A particular example of a wild-type Cas9 amino acid sequence is SEQ ID NO: 25. In some aspects, the CaCas9 nucleotide sequence encodes a Cas9 protein having nuclease activity. In one aspect, a Cas9 protein having nuclease activity comprises SEQ ID NO: 25.

In other aspects, the CaCas9 nucleotide sequence encodes a Cas9 protein that is lacking nuclease activity, also referred to herein as a “nuclease-inactive Cas9 protein”. A nuclease-inactive Cas9 protein can be prepared, for example, by substituting amino acid residues that are required for catalytic activity in a wild type Cas9 protein with a different amino acid(s). For example, the aspartate at position 10 and the histidine at position 840 in the Cas9 protein represented by SEQ ID NO: 25 can be substituted with a different amino acid (e.g., alanine) to yield a nuclease-inactive Cas9. Preferably, the substitutions are non-conservative substitutions. In a particular aspect, a nuclease-inactive Cas9 protein comprises SEQ ID NO: 26. In a particular aspect, the CaCas9 nucleotide sequence encoding the nuclease-inactive Cas9 comprises SEQ ID NO: 29.

Nuclease-inactive CaCas9 encoding mutant nucleotide sequence-codon optimized CaCas9

ATGGATAAAA AGTATAGTAT TGGTTTAGCT ATTGGTACTA ACTCTGTGGG
TTGGGCAGTT ATCACCGACG AATATAAAGT TCCATCAAAG AAATITAAGG
TGTTAGGTAA CACTGACAGA CACTCAATAA AAAAGAATCT TATCGGTGCT
CTTTTGTTCG ACTCCGGTGA AACTGCCGAG GCTACACGTT TAAAAAGAAC
AGCAAGAAGA AGATATACCC GTAGAAAAAA TAGAATATGT TATTTACAAG
AAATCTTTTC TAATGAAATG GCTAAAGTTG ATGATTCCTT TTTCCATAGA
TTGGAAGAGT CATTTTTGGT TGAAGAAGAC AAAAAGCATG AGAGACATCC
AATCTTTGGG AATATAGTTG ATGAAGTGGC TTACCATGAA AAATATCCTA
CCATTTATCA TTTAAGAAAG AAATTGGTAG ATTCAACTGA TAAAGCTGAC
CTTAGATTAA TCTATTTAGC ACTTGCCCAT ATGATTAAAT TTAGAGGTCA
TTTTTTGATT GAAGGTGATT TGAACCCAGA TAATTCTGAC GTGGATAAAT
TATTTATTCA ATTAGTCCAA ACCTACAACC AATTATTTGA GGAAAATCCA
ATTAATGCTA GTGGTGTCGA TGCCAAAGCT ATATTATCAG CCAGATTATC
AAAATCTAGA CGTTTGGAAA ATTTGATTGC CCAATTGCCA GGAGAAAAAA
AGAATGGATT ATTTGGAAAC TTGATCGCAT TATCATTGGG TTTGACACCA
AATTTTAAAT CTAATTTTGA TTTAGCTGAA GATGCTAAAT TACAATTATC
AAAAGACACC TATGACGACG ATTTGGACAA TTTACTTGCT CAAATTGGTG
ATCAATATGC AGATTTGTTC TTAGCTGCTA AAAACTTATC TGATGCTATT
TTGTTGTCTG ATATTTTGAG AGTGAACACA GAAATAACCA AAGCTCCATT
ATCAGCATCT ATGATCAAAC GTTATGATGA ACACCATCAG GATTTGACTT
TATTGAAAGC TTTGGTGAGA CAACAATTGC CAGAGAAGTA TAAAGAAATC
TTTTTCGATC AATCTAAAAA CGGGTATGCA GGTTATATTG ATGGGGGTGC
CTCCCAAGAG GAATTTTACA AATTTATAAA ACCTATTTTA GAAAAGATGG
ATGGGACTGA GGAACTTTTG GTCAAATTGA ACAGAGAAGA TTTGTTACGT
AAACAGAGAA CTITTGATAA TGGTAGTATA CCTCACCAAA TTCATTTGGG
TGAGTTGCAT GCAATTTTAA GAAGACAAGA AGATTTTTAT CCATTTTTAA
AAGATAATAG AGAAAAAATC GAGAAAATTT TAACCTTTAG AATTCCATAC
TATGTTGGGC CTTTGGCTAG AGGTAATTCA AGATTTGCCT GGATGACACG
TAAATCAGAA GAAACTATTA CCCCTTGGAA TTTTGAAGAG GTTGTTGATA
AAGGAGCATC AGCACAGAGT TTTATTGAAA GAATGACCAA TTTCGATAAA
AACTTACCAA ATGAAAAAGT TTTACCAAAA CATTCCTTGT TATACGAATA
TTTTACTGTT TACAATGAAC TTACAAAGGT TAAATATGTT ACTGAAGGTA
TGCGTAAGCC AGCCTTTTTA TCTGGAGAAC AGAAAAAGGC AATAGTTGAT
TTATTGTTTA AAACAAATAG AAAAGTTACT GTTAAACAAT TAAAAGAAGA
TTACTTTAAG AAAATTGAAT GTTTTGATTC AGTTGAAATC AGTGGTGTTG
AAGACAGATT TAATGCTAGT TTAGGAACTT ACCATGATTT ACTTAAAATT
ATCAAAGATA AAGATTTCTT GGATAACGAA GAAAATGAAG ACATTTTAGA
AGACATTGTT TTAACCTTAA CTTTATTCGA AGATAGAGAG ATGATTGAAG
AACGTTTGAA GACTTATGCA CATTTGTTTG ACGATAAAGT GATGAAACAG
TTGAAAAGAA GACGTTATAC TGGATGGGGT AGATTGTCTC GTAAATTGAT
CAATGGAATT AGAGATAAAC AAAGTGGTAA AACTATCTTG GACTTTTTGA
AATCTGACGG ATTTGCTAAT AGAAATTTCA TGCAATTGAT CCACGACGAT
AGTTTGACAT TTAAAGAAGA CATCCAAAAG GCCCAAGTGA GTGGGCAAGG
TGATTCATTA CATGAACATA TTGCAAATTT AGCCGGATCT CCTGCTATTA
AGAAAGGGAT ATTACAAACT GTTAAAGTTG TGGATGAATT AGTGAAAGTA
ATGGGAAGAC ATAAACCTGA AAACATTGTC ATTGAGATGG CAAGAGAAAA
TCAAACTACA CAAAAAGGAC AGAAAAATAG TAGAGAACGT ATGAAAAGAA
TAGAAGAGGG TATTAAAGAA TTGGGTAGTC AAATATTGAA AGAACACCCA
GTGGAAAATA CCCAGTTGCA AAATGAAAAA TTATATCTTT ACTACCTTCA
AAATGGACGT GATATGTATG TTGATCAGGA ATTAGATATA AATAGACTTT
CAGATTATGA TGTAGATGCA ATAGTTCCAC AATCTITTCTT GAAAGATGAT
TCCATAGACA ATAAAGTATT AACTAGAAGT GATAAAAATA GAGGTAAAAG
TGATAATGTC CCAAGTGAGG AAGTCGTCAA AAAGATGAAA AATTACTGGC
GTCAACTTTT GAATGCTAAA TTAATTACTC AAAGAAAATT TGATA ATTTG
ACTAAAGCAG AAAGAGGTGG GCTTTCTGAA TTAGATAAAG CCGGGTTCAT
TAAAAGACAA TTGGTCGAAA CTAGACAAAT TACTAAACAT GTTGCCCAAA
TTTTAGATTC CCGTATGAAC ACTAAGTATG ACGAAAATGA TAAGTTAATA
CGTGAGGTTA AAGTCATTAC TTTAAAATCA AAACTTGTCT CTGATTTCAG
AAAGGATTTC CAATTCTATA AAGTTAGAGA AATTAATAAT TATCATCATG
CTCATGATGC ATATTTGAAT GCTGTAGTTG GAACTGCTTT AATCAAGAAA
TACCCTAAAT TAGAATCTGA ATTTGTATAT GGTGATTACA AAGTCTATGA
TGTTAGAAAG ATGATTGCTA AATCAGAACA AGAAATTGGT AAAGCTACAG
CTAAATACTT CTTTTACTCT AACATTATGA ATTTCTTTAA AACAGAAATT
ACTTTGGCAA ACGGTGAAAT TAGAAAAAGA CCTCTTATTG AAACAAATGG
TGAGACTGGA GAGATAGTTT GGGACAAAGG GCGTGATTTC GCTACTGTTA
GAAAAGTTTT ATCAATGCCA CAAGTTAACA TTGTAAAGAA AACAGAGGTT
CAAACTGGTG GTTTCTCAAA AGAAAGTATT TTGCCTAAAA GAAATAGTGA
TAAATTGATT GCCAGAAAAA AGGATTGGGA TCCAAAGAAA TATGGTGGTT
TCGACTCACC AACCGTAGCC TATTCTGTTT TGGTTGTGGC AAAGGTTGAA
AAGGGTAAAA GTAAAAAGCT TAAATCAGTA AAAGAACTTT TGGGTATTAC
AATAATGGAA AGAAGTTCCT TTGAAAAGAA CCCTATTGAT TTTTTGGAAG
CTAAAGGTTA TAAGGAAGTA AAGAAGGACT TAATAATCAA ATTGCCTAAA
TATTCTTTAT TTGAATTAGA AAATGGGAGA AAAAGAATGT TGGCTTCTGC
TGGAGAATTG CAAAAGGGTA ATGAATTAGC ATTGCCTTCC AAATATGTTA
ACTTCTTGTA TTTAGCTTCA CACTATGAAA AGTTGAAAGG GTCACCAGAA
GATAACGAGC AAAAACAATT ATTTGTTGAA CAACACAAAC ACTACTTAGA
TGAGATTATA GAACAAATTA GTGAATTCAG TAAAAGAGTG ATATTAGCTG
ATGCAAATTT AGATAAAGTT TTGTCAGCCT ATAACAAACA TAGAGATAAG
CCAATTAGAG AACAAGCAGA AAACATTATT CACTTATTTA CCCTTACCAA
TTTAGGAGCA CCTGCTGCTT TCAAGTATTT TGATACAACA ATTGATCGTA
AAAGATATAC CTCAACAAAA GAAGTCTTAG ACGCCACCTT AATTCATCAA
TCAATCACTG GATTGTATGA GACAAGAATT GATTTGTCTC AATTGGGTGG
TGATGAAGGG GCT (SEQ ID NO: 29)

Methods for performing site-directed mutagenesis to produce proteins having amino acid substitutions are well known and routine to one of ordinary skill in the art In certain aspects, the CaCas9 nucleotide sequence encodes a Cas9 protein fragment that lacks nuclease activity.

In certain aspects, the nuclease-inactive Cas9 protein is expressed as a fusion protein with all or a portion of a heterologous protein that represses gene transcription, also referred to herein as a “repressor” protein. Numerous repressor proteins that can be readily adapted for the present technology are known in the art. In one aspect, the nuclease-inactive Cas9 is fused to a Candida albicans suppressor of Snf1 6 (SSN6) protein (SEQ ID NO: 30).

MYATAHTIKQ QQQQQQQHPP PPLNGGLHAS GAPPNSHEAA AIAQQQQQQQ
QHHNGPGMIV AAAAASANQQ AVQARAQQQQ QQQQQRLPSS AALNETTVST
WLAIGSLAES LGDIERATAS YNSALRHSPN NPDILVKIAN TYRSKDQFLK
AAELYEQALN FHVENGETWG LLGHCYLMLD NLQRAYAAYQ RALFYLENPN
VPKLWHGIGI LYDRYGSLEY AEEAFVRVLD LDPNFDKANE IYFRLGIIYK
HQGKLQPALE CFQYILNNPP HPLTQPDVWF QIGSVYEQQK DWNGAKDAYE
KVLQINPHHA KVLQQLGCLY SQAESNPSTP ANGAAPPHKP FQQDLTIALK
YLKQSLEVDQ SDAHSWYYLG RVEMIRGDFT AAYEAFQQAV NRDARNPTFW
CSIGVLYYQI SQYRDALDAY TRAIRLNPYI SEVWYDLGTL YETCNNQISD
ALDAYRQAER LDPNNPHIKA RLEQLTKYQQ EGNTHPPQPP PSSQQPRLPQ
GMVLESTQQQ QQQQPPPPPQ QQQQQLQHQS QSQPQPQQPP QTQSQPSLLQ
HQSSLPPQQI QPLHQQAAKP LVNQQQSPPP PHLMNLGQPG QQPQQLPPHL
PPHTQQPSQI QEKPPTQEQP HYQPPPPPQH QQQSQSQPQP PHQPQHTQNQ
SPQLAQLPPH HSNPPAKPHG APQQRTGLPD LLHNSANIIS APSQVPQPQQ
QYQQPHIAPV RQEQVNHVPS IYSAPRPTET TLPQINNPNE STTTQVPQLK
KEEPKPEATV SAPVPEAIKV QDQVTIQESA PAAAAAVSAP ASAPVGDIKT
DTVSTTTPAT STTADAVPVS VSQVGEAPNV VQEKKVPDTE QIVSQVEKPV
ESQPEVTPAP TPAPALATAP TEPAPTDKDV VMAPSKSATP VPQSIVEQNT
RVSEATKAPE SNGKHDLEDK NDEEKILKRP TVETTTESVP VNQPVEKENE
KVEVPPPSEQ PSSEKREKEV NGSIKKPLEN ESKVDIPQFS SNITAQNEEA
KSGEETKKDT TKTSPAKQGE VKEVIPSSTE TVSKPDVEKD NKEKDKDEDE
VMADEDDVKK DENPEPPMRK IEEDENYDDE (SEQ ID NO. 30)

In other aspects, the nuclease-inactive Cas9 protein is expressed as a fusion protein with all or a portion of a heterologous protein that activates gene transcription, also referred to herein as an “activator” protein. Numerous activator proteins that can be readily adapted for the present technology are known in the art. For example, at least two tandem copies (e.g., 4 or more copies) of a fragment (DALDDFDLDML (SEQ ID NO: 31)) derived from transcription activator VP16 can be adapted for use in the present technology (Seipel et al., Biol. Chem, Hoppe-Seyler, 375(7):463-70, 1994). Other examples of transcription activators include GAL4 and GCN4.

In some aspects, the CaCas9 nucleotide sequence encodes a Cas9 protein having a nickase activity, also referred to herein as a “Cas9 nickase”. A Cas9 nickase, which can nick one strand of a double-stranded nucleic acid, facilitates homology-directed repair in eukaryotic cells (Cong, et al., Science, 339, 819-23, 2013). A Cas9 nickase can be prepared, for example, by substituting amino acid residues that are required for catalytic activity in a wild-type Cas9 protein with a different amino acid(s). For example, a single substitution of the aspartate at position 10, the glutamic acid at position 762, the histidine at position 840, the asparagine at position 863, the histidine at position 983, or the aspartic acid at position 986 in the Cas9 protein represented by SEQ ID NO: 25 can be substituted with a different amino acid (e.g., alanine) to yield a Cas9 nickase (see, e.g., Nishimasu, et al., Cell, 156:935-49, 2014). Preferably, the substitutions are non-conservative substitutions. Methods for producing proteins having amino acid substitutions (e.g., site-directed mutagenesis) are well known and routine to one of ordinary skill in the art.

In other aspects, the CaCas9 nucleotide sequence encodes a Cas9 protein having a relaxed requirement for the NGG sequence, referred to herein as “CaCas9-PAM”. Cas9 directs cleavage at sites in the genome which match the appropriate region specified by the sgRNA when they are followed by the sequence NGG. Substituting two amino acids-arginine at position 1333 and arginine at position 1335 of SEQ ID NO: 25-relaxes the requirement for the NGG sequence, otherwise known as the PAM. By removing this requirement, the potential targeting applications are greatly increased. Preferably, the substitution is a non-conservative substitution. In one aspect, R1333 and R1335 are substituted with glutamine. In certain aspects, the substitutions in CaCas9-PAM may be combined with the substitutions in the nuclease-inactive CaCas9-SSN6 to create a repressor which can target a much larger array of sequences. In other aspects, the substitutions in CaCas9-PAM may be combined with the substitutions in the nuclease-inactive CaCas9 fused to a transcription activator to create a gene activator which can target a much larger array of sequences. In various aspects, the substitutions in CaCas9-PAM may be combined with any one of the Cas9 nickase substitutions described herein.

In some aspects, a nucleic acid comprising a CaCas9 nucleotide sequence further comprises a nucleotide sequence encoding a heterologous peptide fused in-frame with the CaCas9 coding sequence. Examples of heterologous peptide sequences that can be fused to a Cas9 protein include nuclear localization sequences, signal peptides and protein tags. In one aspect, a nucleic acid comprising a CaCas9 nucleotide sequence further comprises a sequence encoding an NLS (e.g., SV40-NLS) fused in-frame with the CaCas9 coding sequence. In a further aspect, a nucleic acid comprising a CaCas9 nucleotide sequence further comprises a sequence encoding protein tag fused in-frame with the CaCas9 coding sequence. As used herein, “tag” refers to a sequence that is useful for, e.g., purifying, expressing, solubilizing, and/or detecting a polypeptide. In certain aspects, a tag can serve multiple functions. Examples of suitable protein tags for the present technology include HA, TAP, MYC, HIS, FLAG, V5, and GST tags. In a particular aspect, the tag comprises SEQ ID NO: 32.

sV40-NLS/FLAG

GATCCTAAGA AGAAAAGAAA AGTTGATCCA AAGAAAAAGC GTAAGGTGGA TCCTAAGAAA AAGAGAAAGG TTGACTACAA AGACCATGAC GGTGATTATA
AAGATCATGA CATCGACTAC AAGGATGACG ATGACAAGTG ATAA (SEQ ID NO: 32)

3×SV40-NLS (underlined)
3×Flag (normal)
2×STOP (italicized)

In various aspects, a nucleic acid comprising a CaCas9 nucleotide sequence further comprises all or a portion of a plasmid (e.g., vector) sequence. For example, a nucleic acid comprising a CaCas9 nucleotide sequence can include one or more plasmid sequences selected from the group consisting of a promoter sequence (e.g., an ENO1, TEF1, MAL2, URA3, ACT1, SAP2, OP4, WH11, MET3, and HWP1 promoter sequence), an antibiotic resistance sequence (e.g., nourseothricin resistance NAT^{R}), an inducible recombination sequence (e.g., FRT sequence), and a locus-targeting sequence (e.g., ENO1, RP10, and NEUTSL) to direct integration of all or a portion of the nucleic acid into a yeast genome. As those of skill in the art would appreciate in light of the present disclosure, more than one promoter sequence can be used For example, a TEF1 promoter sequence can be inserted downstream of, e.g., an ENO1 promoter. In some embodiments, the locus-targeting sequence targets the CRISPR system to an intergenic space (e.g., the Neut5L locus).

In some embodiments, the plasmid comprises a Cre/Lox recombination sequence. In one embodiment, a dominant resistance marker sequence is used. In some embodiments, the yeast strain is a prototroph. In some embodiments, the yeast strain is an auxotroph. In some embodiments, as described herein, the promoter sequence is specific for the yeast system used to, e.g., enhance expression. For example, a S. cerevisiae TEF1 promoter is used if expressing in the S. cerevisiae system. Similarly, a promoter, e.g. TEF1 specific to Naumovozyma castellii is used if expressing in the Naumovozyma castellii system.

In some aspects, a nucleic acid comprising a CaCas9 nucleotide sequence also comprises a synthetic guide RNA (sgRNA) coding sequence. For example, the sgRNA coding sequence can be designed to express an sgRNA molecule targeting one or more target gene sequences. Thus, a variety of target sequences in a yeast genome can be modified using the present Candida-compatible CRISPR/Cas9 system.

As used herein, to “modify” a nucleic acid (e.g., a genome, a target gene, a target sequence) means to alter, or mutate, the nucleotide sequence of the nucleic acid, for example, by replacement (e.g., substitution), introduction, and/or deletion of one or more nucleotides in the nucleic acid.

In some aspects, a single sgRNA sequence can be complementary to one or more (e.g., all) of the target nucleic acid sequences that are being modified. In one aspect, a single sgRNA is complementary to a single target nucleic acid sequence. In a particular aspect in which two or more target nucleic acid sequences are to be modified, multiple sgRNA sequences (or sgRNA coding sequences) can be introduced. wherein each sgRNA sequence is complementary to (specific for) one target nucleic acid sequence. In other aspects, a single sgRNA sequence is complementary to at least two targets or more (all) of the target nucleic acid sequences.

Each sgRNA sequence can vary in length from about 8 base pairs (bp) to about 200 bp. In some aspects, the sgRNA sequence can be about 9 to about 50 bp; about 10 to about 40 bp; about 12 to about 30; about 14 to about 28; about 15 to about 25; about 16 to about 24, about 17 to about 23; about 18 to about 22; about 19 to about 21 bp in length.

The portion of each target nucleic acid sequence to which each sgRNA sequence is complementary can also vary in size. In particular aspects, the portion of each target nucleic acid sequence to which the sgRNA is complementary can be about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 39, 40, 41, 42, 43, 44, 45, 46 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80 81, 82, 83, 84, 85, 86, 87 88, 89, 90, 81, 92, 93, 94, 95, 96, 97, 98, or 100 nucleotides (contiguous nucleotides) in length. In some embodiments, each sgRNA sequence can be at least about 70%, 75%, 80%, 85%, 90%, 95%, 100% etc. identical or similar to the portion of each target nucleic acid sequence. In some embodiments, each sgRNA sequence is completely or partially identical or similar to each target nucleic acid sequence For example, each RNA sequence can differ from perfect complementarity to the portion of the target sequence by about 1, 2, 3, 4, 5, 6, 7, 8, 9, 10, 11, 12, 13, 14, 15, 16, 17, 18, 19, 20, etc., nucleotides. In some embodiments, one or more sgRNA sequences are perfectly complementary (100%) across at least about 10 to about 25 (e.g., about 20) nucleotides of the target nucleic acid

In one embodiment, the sgRNA coding sequence encodes an sgRNA that targets one or more genes associated with high immune cell-damaging capacity including, but not limited to ECE1, UME6, FLO8, EFG1, or CPH1.

In one aspect, the sgRNA coding sequence is operably linked to a promoter (e.g., a different promoter than the promoter that controls expression of the CaCas9 sequence). A variety of suitable promoters for use in the present technology are known in the art. In a particular aspect, the promoter is a yeast RNA polymerase III promoter (e.g., a Candida albicans SNR52 promoter, or RDN5 promoter). In some embodiments, as described herein, the promoter sequence can be specific for the yeast system used. Thus, for example, a promoter operably linked to an sgRNA coding sequence allows for the expression of the sgRNA, which affects targeting of the CRISPR/Cas system to a gene of interest (e.g., the target gene), to enable modification of the target gene

In other aspects, the present technology relates to a nucleic acid for delivering an sgRNA coding sequence. The nucleic acid for delivering an sgRNA coding sequence can include, for example, a promoter (e.g., an RNA polymerase III promoter), a cloning site for introducing an sgRNA coding sequence, and/or a locus-targeting sequence to direct integration of all or a portion of the nucleic acid into a yeast genome (e.g., a yeast RP10 sequence). In some aspects, the nucleic acid for delivering an sgRNA coding sequence comprises a synthetic guide RNA (sgRNA) coding sequence. For example, the sgRNA coding sequence can be designed to express an sgRNA molecule targeting one or more of the sequences provided herein using routine knowledge and skills possessed by one of ordinary skill in the art. As will be appreciated by those of skill in the art in light of the present disclosure, the sgRNA can be delivered as a DNA molecule (e.g., as nucleic acid encoding the desired sgRNA) or an RNA molecule

In some aspects, the nucleic acid for delivering an sgRNA coding sequence includes an RNA polymerase III promoter. In a particular aspect, the RNA polymerase III promoter is a yeast (e.g., Candida albicans) SNR52 promoter.

In other aspects, the nucleic acid for delivering an sgRNA coding sequence includes a yeast (e.g., Candida albicans) RP10 sequence as a locus-targeting sequence.

In various aspects, a nucleic acid for delivering an sgRNA coding sequence further comprises all or a portion of a plasmid (e.g., vector) sequence. For example, a nucleic acid for delivering an sgRNA coding sequence can include an antibiotic resistance sequence (e.g., a sequence that confers resistance to nourseothricin (Nat)). A variety of suitable plasmids and plasmid sequences suitable for use in the present technology are known in the art (Celik E and Calik P, Biotechnol Adv. 30(5):1 108-18, 2011).

### Methods of Producing Genetically-Modified C Albicans Strains That Reside in Human Gut Tissue Using Candida-Compatible Nucleic Acids Encoding CRISPR/Cas9 System Components

In yet another aspect, the present technology provides a method for modifying a genome of a C. albicans strain that resides in human gut tissue. The method generally comprises the steps of: a) introducing into a cell of the C. albicans strain a first nucleic acid comprising a Candida-compatible clustered regularly interspaced short palindromic repeat (CRISPR)-associated nuclease 9 (CaCas9) nucleotide sequence that encodes a protein sequence having at least 90% sequence identity to SEQ ID NO: 25, or a fragment thereof, wherein each leucine in the protein is encoded by a codon other than CTG or CUG; b) introducing into the cell of the C. albicans strain a second nucleic acid comprising an sgRNA coding sequence; and c) expressing the CaCas9 and sgRNA coding sequences in the cell of the C. albicans strain, thereby modifying the genome of the cell of the C. albicans strain. Methods of introducing nucleic acids (e.g., plasmids) into cells (e.g., C. albicans cells) are well known in the art and include, for example, routine methods for transforming yeast cells (e.g., by electroporation).

Suitable first nucleic acids (e.g., DNA or RNA) comprising a CaCas9 nucleotide sequence for use in the methods of the present technology include, for example, the various nucleic acids comprising a CaCas9 nucleotide sequence disclosed herein.

Suitable second nucleic acids (e.g., DNA or RNA) comprising an sgRNA coding sequence for use in the methods of the present technology include, for example, the various nucleic acids comprising an sgRNA coding sequence disclosed herein. In certain aspects, the second nucleic acid bound to (e.g., in a complex with) a Cas9 protein, or fragment thereof is introduced into the cell of the C. albicans strain.

In some aspects, the method further comprises introducing into the cell of the C. albicans strain a heterologous repair template nucleic acid sequence. As used herein, a “heterologous repair template nucleic acid sequence” refers to a nucleic acid sequence that is complementary to a portion of a target nucleic acid sequence that is cleaved by a Cas (e.g., Cas9) protein. A variety of nucleic acid sequences can be included in a repair template, including, e.g., a single-stranded oligonucleotide, a double-stranded oligonucleotide, a plasmid, a cDNA, a gene block (e.g., gBlocks™ Gene Fragments (IDT)), a PCR product, and the like. Thus, the size of the nucleic acid sequences can vary and will depend upon the reason for introducing the nucleic acid sequence.

For example, the one or more nucleic acid sequences can be used to replace one or more nucleotides, introduce one or more additional nucleotides, delete one or more nucleotides or a combination thereof in the target nucleic acid sequences. In a particular aspect, the heterologous repair template nucleic acid introduces a point mutation in the target sequences. In another aspect, the heterologous repair template nucleic acid replaces a mutant nucleotide with a wild-type nucleotide in the target sequences. In other aspects, the heterologous repair template nucleic acid may introduce a tag (e.g., a fluorescent protein such as green fluorescent protein), label and/or cleavage site. Thus, the heterologous repair template nucleic acid sequence can be from about 10 nucleotides to about 5000 nucleotides, about 20 to 4500 nucleotides, about 30 to 4000 nucleotides, about 50 to 3500 nucleotides, about 60 to about 3000 nucleotides, about 70 to about 2500 nucleotides, about 80 to about 2000 nucleotides, about 90 to about 1500 nucleotides, about 100 to about 1000 nucleotides, etc. In a particular aspect, the heterologous repair template nucleic acid is about 10 to about 500 nucleotides. In a particular aspect, the heterologous repair template nucleic acid sequence (e.g., oligonucleotide) is used to further modify (alter, edit, mutate) the cleaved target nucleic acid sequence (e.g., such oligo-mediated repair allows for precise genome editing). As will be apparent to those of skill in the art, a variety of methods for introducing nucleic acid into a yeast cell are well known and routine.

In certain aspects of the method, the first nucleic acid, and the second nucleic acids, or both, are introduced into the cell of the C. albicans strain on a plasmid. In one aspect, the first nucleic acid and the second nucleic acid are introduced into the cell of the C. albicans strain on a single plasmid. As described herein, the single plasmid may comprise an sgRNA coding sequence to express an sgRNA that targets a variety of sequences in a C. albicans genome, depending upon the desired results. For example, the sgRNA may target one or more of the sequences provided herein using routine knowledge and skills possessed by one of ordinary skill in the art.

In further aspects of the method, the first and second nucleic acids are introduced into the cell of the C. albicans strain on two different plasmids, in no preferred order.

In certain aspects, the first and second nucleic acids are integrated in the genome of the cell of the C. albicans strain. In general, once the first and second nucleic acids are integrated into the cell’s genome, the nucleic acids are expressed to produce Cas9 protein and sgRNA that can function collectively to edit the cell’s genome.

### Therapeutic Methods of the Present Technology

In one aspect, the present disclosure provides a method for treating a patient suffering from a fungal-associated intestinal inflammatory disorder comprising administering to the patient an effective amount of an IL-1 pathway inhibitor, wherein gut tissue of the patient comprises a population of candidalysin-secreting C. albicans.

In one aspect, the present disclosure provides a method for selecting a patient suffering from a fungal-associated intestinal inflammatory disorder for treatment with an IL-1 pathway inhibitor comprising (a) detecting the presence of candidalysin-secreting C. albicans in a biological sample obtained from the patient; and (b) administering to the patient an effective amount of an IL-1 pathway inhibitor. In some embodiments, the biological sample is a colonic mucosa-enriched lavage sample, a fecal sample, a rectal swab, or an intestinal sample.

In some embodiments, the presence of candidalysin-secreting C. albicans in the biological sample is assayed via next-generation sequencing, PCR, real-time quantitative PCR (qPCR), digital PCR (dPCR), Southern blotting, Reverse transcriptase-PCR (RT-PCR), Northern blotting, microarray, dot or slot blots, in situ hybridization, or fluorescent in situ hybridization (FISH).

In certain embodiments, candidalysin mRNA expression levels are detected via real-time quantitative PCR (qPCR), digital PCR (dPCR), Reverse transcriptase-PCR (RT-PCR), Northern blotting, microarray, dot or slot blots, in situ hybridization, or fluorescent in situ hybridization (FISH). In other embodiments, candidalysin polypeptide expression levels are detected via Western blotting, enzyme-linked immunosorbent assays (ELISA), dot blotting, immunohistochemistry, immunofluorescence, immunoprecipitation, immunoelectrophoresis, or mass-spectrometry.

Additionally or alternatively, in some embodiments of the methods disclosed herein, the fungal-associated intestinal inflammatory disorder is inflammatory bowel disease (IBD), Crohn’s disease (CD), or ulcerative colitis (UC).

In any and all embodiments of the methods disclosed herein, the IL-1 pathway inhibitor is an inflammasome-blocking drug, an anti-IL-1R1 antibody or antigen binding fragment, Anakinra, Rilonacept, Canakinumab, Gevokizumab, LY2189102, MABp1, MEDI-8968, CYT013, sIL-1RI, sIL-1RII, EBI-005, CMPX-1023, MCC950, Inzomelid, Somalix, NT-0167, IFM-2427 (DFV890), Dapansutrile (OLT1177), glyburide, 16673-34-0, JC124, FC11A-2, parthenolide, Bay 11-7082, BHB, MNS, CY-09, tranilast, oridonin, VX-740, or VX-765.

Additionally or alternatively, in some embodiments of the methods disclosed herein, the candidalysin-secreting C. albicans expresses elevated enhanced filamentous growth protein 1 (EFG1) expression compared to a reference non-filamentous C. albicans strain or a predetermined threshold. In certain embodiments, the candidalysin-secreting C. albicans expresses increased hyphae production relative to a reference non-filamentous C. albicans strain. Additionally or alternatively, in certain embodiments of the methods disclosed herein, the candidalysin-secreting C. albicans expresses elevated expression levels of at least one protease selected from among SAP6, SAP5, or SAP2 compared to a reference non-filamentous C. albicans strain or a predetermined threshold. In other embodiments of the methods disclosed herein, the candidalysin-secreting C. albicans expresses elevated expression levels of ALS3 or ALS1 compared to a reference non-filamentous C. albicans strain or a predetermined threshold. In any of the preceding embodiments of the methods disclosed herein, the reference non-filamentous C. albicans strain is an efg1Δ/Δ C. albicans mutant strain.

Additionally or alternatively, in some embodiments of the methods disclosed herein, the candidalysin-secreting C. albicans induces an in vivo proinflammatory response in host cells. In some embodiments, the in vivo proinflammatory responses comprise neutrophil infiltration and/or Th17 responses in the colon of the patient.

In certain embodiments, the human subject is diagnosed with or is suffering from inflammatory bowel disease (IBD), Crohn’s disease (CD), or ulcerative colitis (UC).

Additionally or alternatively, in some embodiments, the at least one C. albicans strain inflicts macrophage damage that is elevated or comparable to C. albicans SC5314. In some embodiments, the at least one C. albicans strain comprises one or more of IDD581, IDA653, IDB311, IDB671, IDB312, IDB313, IDB831, IDB071, IDB072, IDB101, IDB104, IDC481, IDC482, IDC483, IDC571, IDC572, IDD582, IDC711, or IDC712.

### Kits of the Present Technology

Also provided herein are kits comprising (a) a first expression vector comprising a nucleic acid sequence encoding a Candida-compatible Cas9 nuclease and a nucleic acid sequence encoding a synthetic guide RNA (sgRNA) that is configured to cleave a region in a target gene of at least one C. albicans strain that resides in human gut tissue, wherein the target gene is associated with high immune cell-damaging capacity and wherein the at least one C. albicans strain induces proinflammatory immunity in a human subject; and (b) a heterologous repair template nucleic acid sequence comprising (i) a 5′ region that is homologous to a C. albicans nucleic acid sequence that is upstream or downstream from the region in the target gene that is cleaved by the sgRNA and (ii) a 3′ region comprising an open reading frame (ORF) deletion of the target gene, wherein the target gene is ECE1, UME6, or FLO8 and instructions for using the same to genetically modify C. albicans isolates that reside in human gut.

Additionally or alternatively, in some embodiments, the 5′ region of the heterologous repair template nucleic acid sequence has a length of about 20-30 base pairs (bps), 30-40 bps, 40-50 bps, 50-60 bps, 60-70 bps, 70-80 bps, 80-90 bps, 90-100 bps, 100-110 bps, 110-120 bps, 120-130 bps, 130-140 bps, 140-150 bps, 150-160 bps, 160-170 bps, 170-180 bps, 180-190 bps, 190-200 bps, 200-210 bps, 210-220 bps, 220-230 bps, 230-240 bps, 240-250 bps, 250-260 bps, 260-270 bps, 270-280 bps, 280-290 bps, 290-300 bps, 300-310 bps, 310-320 bps, 320-330 bps, 330-340 bps, 340-350 bps, 350-360 bps, 360-370 bps, 370-380 bps, 380-390 bps, 390-400 bps, 400-410 bps, 410-420 bps, 420-430 bps, 430-440 bps, 440-450 bps, 450-460 bps, 460-470 bps, 470-480 bps, 480-490 bps, 490-500 bps, 500-510 bps, 510-520 bps, 520-530 bps, 530-540 bps, 540-550 bps, 550-560 bps, 560-570 bps, 570-580 bps, 580-590 bps, or 590-600 bps. In some embodiments, the 5′ region of the heterologous repair template nucleic acid sequence is about 60 base pairs in length.

Additionally or alternatively, in some embodiments, the 3′ region of the heterologous repair template nucleic acid sequence has a length of about 20-30 base pairs (bps), 30-40 bps, 40-50 bps, 50-60 bps, 60-70 bps, 70-80 bps, 80-90 bps, 90-100 bps, 100-110 bps, 110-120 bps, 120-130 bps, 130-140 bps, 140-150 bps, 150-160 bps, 160-170 bps, 170-180 bps, 180-190 bps, 190-200 bps, 200-210 bps, 210-220 bps, 220-230 bps, 230-240 bps, 240-250 bps, 250-260 bps, 260-270 bps, 270-280 bps, 280-290 bps, 290-300 bps, 300-310 bps, 310-320 bps, 320-330 bps, 330-340 bps, 340-350 bps, 350-360 bps, 360-370 bps, 370-380 bps, 380-390 bps, 390-400 bps, 400-410 bps, 410-420 bps, 420-430 bps, 430-440 bps, 440-450 bps, 450-460 bps, 460-470 bps, 470-480 bps, 480-490 bps, 490-500 bps, 500-510 bps, 510-520 bps, 520-530 bps, 530-540 bps, 540-550 bps, 550-560 bps, 560-570 bps, 570-580 bps, 580-590 bps, or 590-600 bps. In other embodiments, the 3′ region of the heterologous repair template nucleic acid sequence is about 20 base pairs in length.

In some embodiments, the kit further comprises a second expression vector comprising a nucleic acid sequence encoding a Candida-compatible Cas9 nuclease and a nucleic acid sequence encoding a synthetic guide RNA (sgRNA) that is configured to cleave a region in EFG1, or CPH1 of the at least one C. albicans strain.

The kits may further comprise one or more primers comprising the sequence of any one of SEQ ID NOs: 1-19.

In some embodiments, the kits further comprise buffers, enzymes having polymerase activity, enzymes having polymerase activity and lacking 5′->3′ exonuclease activity or both 5′->3′ and 3′->5′ exonuclease activity, culture media, enzyme cofactors such as magnesium or manganese, salts, chain extension nucleotides such as deoxynucleoside triphosphates (dNTPs), modified dNTPs, nuclease-resistant dNTPs or labeled dNTPs, necessary to carry out an assay or reaction, such as amplification and/or engineering alterations (e.g., knock-in or knock-out alterations) in target nucleic acid sequences corresponding to specific fungal genes that are associated with high immune cell-damaging capacity in human subjects.

In one embodiment, the kits of the present technology further comprise a positive control nucleic acid sequence and a negative control nucleic acid sequence to ensure the integrity of the assay during experimental runs. A kit may further contain a means for comparing the levels and/or activity of one or more fungal genes associated with high immune cell-damaging capacity (e.g., ECE1, UME6, FLO8, EFG1, or CPH1) in a sample obtained from a subject with a control sample.

The kits of the present technology can also include other necessary reagents to perform any of the NGS techniques disclosed herein. For example, the kit may further comprise one or more of: adapter sequences, barcode sequences, reaction tubes, ligases, ligase buffers, wash buffers and/or reagents, hybridization buffers and/or reagents, labeling buffers and/or reagents, and detection means. The buffers and/or reagents are usually optimized for the particular amplification/detection technique for which the kit is intended. Protocols for using these buffers and reagents for performing different steps of the procedure may also be included in the kit.

Methods of extracting nucleic acids from samples are well known in the art and can be readily adapted to obtain a sample that is compatible with the system utilized. Automated sample preparation systems for extracting nucleic acids from a test sample are commercially available, e.g., Roche Molecular Systems’ COBAS AmpliPrep System, Qiagen’s BioRobot 9600, and Applied Biosystems’ PRISM™ 6700 sample preparation system.

Also disclosed herein are kits comprising reagents for detecting the presence of candidalysin-secreting C. albicans in a biological sample (e.g., primers, probes, anti-candidalysin antibodies) obtained from a patient suffering from a fungal-associated intestinal inflammatory disorder, and instructions for using one or more IL-1 pathway inhibitors to treat the same. In some embodiments, the kits of the present technology further comprise IL-1 pathway inhibitors. Examples of IL-1 pathway inhibitors include, but are not limited to, an inflammasome-blocking drug, an anti-IL-1R1 antibody or antigen binding fragment, Anakinra, Rilonacept, Canakinumab, Gevokizumab, LY2189102, MABp1, MEDI-8968, CYT013, sIL-1RI, sIL-1RII, EBI-005, CMPX-1023, MCC950, Inzomelid, Somalix, NT-0167, IFM-2427 (DFV890), Dapansutrile (OLT1177), glyburide, 16673-34-0, JC124, FC11A-2, parthenolide, Bay 11-7082, BHB, MNS, CY-09, tranilast, oridonin, VX-740, or VX-765. In some embodiments, the biological sample is a colonic mucosa-enriched lavage sample, a fecal sample, a rectal swab, or an intestinal sample.

Typically, the kits are compartmentalized for ease of use and can include one or more containers with reagents. In one embodiment, all of the kit components are packaged together. Alternatively, one or more individual components of the kit can be provided in a separate package from the other kits components. The kits can also include instructions for using the kit components.

### EXAMPLES

The present technology is further illustrated by the following Examples, which should not be construed as limiting in any way.

### Example 1: Materials and Methods

### Mucosa-Enriched Lavage Samples

Seventy-eight colonic mucosa-enriched lavage samples (38 non-IBD individuals and 40 patients with ulcerative colitis) were obtained following Institutional-Review-Board-approved protocols from the JRI IBD Live Cell Bank Consortium at Weill Cornell Medicine. Samples were collected, processed and stored as previously described^{60,61}. All subjects from which the samples originated were free of anti-fungal treatment.

### Mice

8-12-week-old wild-type (WT) C57BL/6J mice were purchased from Jackson laboratory with an approximately equal gender ratio. All animals were kept under specific pathogen-free conditions (Helicobacter pylori free) and raised on a 12-hour light/dark cycle with access to water and food (5053 Rodent Diet 20, PicoLab) ad libitum. Germ-free (GF) WT mice were bred and maintained within sterile vinyl isolators at Weill Cornell Medical College Gnotobiotic Mouse Facility. Altered Schaedler flora (ASF) mice were generated from germ-free WT C57BL/6J mice upon inoculation with ASF community⁴⁰, bred for at least 5 generations for fully immunocompetent progeny and maintained within sterile vinyl isolators at Weill Cornell Medical College Gnotobiotic Mouse Facility. All animal experiments were approved and are in accordance with the Institutional Animal Care and Use Committee guidelines at Weill Cornell Medicine.

### Fungal Colonization in Vivo

8-10-week-old GF and ASF WT C57BL/6J mice were orally gavaged with C. albicans (1 × 10⁸ CFU/mouse) or Pichia kudriavzevii (1 × 10⁸ CFU/mouse, pkID01) at day 1 and day 2. After fungal colonization, mice were maintained within sterile vinyl isolators for 21 days and then sacrificed at day 23. All C. albicans strains used in these experiments were cultured in Sabouraud dextrose broth (SDB) and washed by sterile PBS twice before use. Fecal samples were collected to confirm the C. albicans colonization by plating on Sabouraud dextrose agar (SDA).

### Murine Models of Colitis and Fungal Colonization

Wild-type C57BL/6 (WT) SPF mice were orally gavaged with C. albicans (1 × 10⁸ CFU/mouse) or or Pichia kudriavzevii (1 × 10⁸ CFU/mouse) twice per week for the duration of the experiments. In a one-cycle of DSS-induced murine model of colitis, WT C57BL/6 SPF mice received prednisolone daily (10 mg/kg/day) or control PBS via intraperitoneal injection for a total of 10 days. After prednisolone treatment, mice were rested for 4 days prior to Dextran sulfate sodium salt (DSS, MP Biomedicals) exposure. To induce colitis, 3% DSS water (w/v) was provided to the mice for 7 days. At day 4 of DSS treatment, mice received another 4 days of daily prednisolone (10 mg/kg/day) treatment. Three days after DSS withdrawal, mice were sacrificed. In a two-cycles of DSS induced colitis model, WT C57BL/6 SPF mice were first given 3% DSS in their drinking water for 7 days. At day 5 of 3% DSS exposure, mice were treated with prednisolone (10 mg/kg/day) for total of 10 days. Mice were rested for 4 days after prednisolone treatment prior to a second 5-day treatment of 3% DSS exposure; mice were sacrificed 3 days after the second DSS treatment. In the IL-1R blockade experiments, 1 mg of InVivoMab anti-IL-1R1 IgG (JAMA147; BioXCell) or 1 mg of InVivoMAb Armenian hamster IgG (BioXcell) were administered via intraperitoneal injection every 4 days for the duration of the experiment.

### Human Gut Fungal Strains Isolation and Culture Conditions

Fecal colonic mucosa-enriched lavages collected from non-IBD or UC-affected subjects were diluted in sterile PBS and plated onto Sabouraud dextrose agar (SDA), supplemented with penicillin/streptomycin (Sigma) and inhibitory mold agar (Hardy Diagnostics). SDA plates were incubated at 37° C. for 48 hours. Inhibitory mold agar plates were incubated at 30° C. for 72 hours. Fungal colonies were picked up from both cultures (37° C., overnight). Isolated fungal colonies from each individual subject were identified by matrix-assisted laser desorption/ionization-time of flight (MALDI-TOF) mass spectrometer.

### DNA Isolation, Mycobiome Library Generation and Sequencing

Mucosa-enriched lavage samples (300 □L) from non-IBD or UC-affected subjects were centrifuged at 400 g. Pellets were collected for fungal DNA isolation. Pellets were treated with 200 U/mL lyticase (Sigma) followed by bead beating, and processing using the Quick-DNA Fungal/Bacterial Kit (Zymo Research) as previously described⁴¹. Fungal DNA presence was validated by RT-PCR for fungal 18S. Based on this approach one low quality sample (out of 78) was excluded and did not proceed for further mycobiome sequencing and analysis. Fungal ITS1-2 regions were amplified by PCR using primers with sample barcodes and sequencing adaptors.

Fungal primers:

ITS1F-CTTGGTCATTTAGAGGAAGTAA (SEQ ID NO: 20)

ITS2R-GCTGCGTTCTTCATCGATGC (SEQ ID NO: 21)

Forward overhang: 5′ TCGTCGGCAGCGTCAGATGTGTATAAGAGACAG-[locus-specific sequence] (SEQ ID NO: 22)

Reverse overhang: 5′ GTCTCGTGGGCTCGGAGATGTGTATAAGAGACAG-[locus-specific sequence] (SEQ ID NO: 23)

ITS amplicons were generated with 35 cycles using Invitrogen AccuPrime PCR reagents (Carlsbad). Amplicons were then used in the second PCR reaction, using Illumina Nextera XT v2 (Illumina) barcoded primers to uniquely index each sample. 2×300 paired-end sequencing was then performed on the Illumina MiSeq (Illumina). DNA was amplified using the following PCR protocol: Initial denaturation at 94° C. for 10 min, followed by 40 cycles of denaturation at 94° C. for 30 s, annealing at 55° C. for 30 s, and elongation at 72° C. for 2 min, followed by an elongation step at 72° C. for 30 min. All libraries were subjected to quality control using DNA 1000 Bioanalyzer (Agilent), and Qubit (Life Technologies) to validate and quantify library construction prior to preparing a Paired-End flow cell. Samples were randomly divided among flow cells to minimize sequencing bias. Clonal bridge amplification (Illumina) was performed using a cBot (Illumina). 2 × 250 bp sequencing-by-synthesis was performed on Illumina MiSeq platform (Illumina).

### ITS1 Fungal Reads

Raw FASTQ ITS1 sequencing data were filtered to enrich for high quality reads, removing the adapter sequence by cutadapt v1.4.1 or any reads that did not contain the proximal primer sequence⁶². Sequence reads were then quality-trimmed by truncating reads not having an average quality score of 20 over a 3-base pair sliding window and removing reads shorter than 100 bp⁶². These high-quality reads were then aligned to Targeted Host Fungi (THF) ITS 1 database, using BLAST v2.2.22 and the pick _otus.py pipeline in the QIIME v1.6 wrapper with an identity percentage >97% for OTU picking⁶³. The alignment results were subsequently tabulated across all reads, using the accession identifier of the ITS reference sequences as surrogate OTUs and using a Perl script¹⁵. Among the analyzed samples, one sample was excluded due to insufficient quality size (ITS reads < 200). Shannon index at the OTU level, NMDS scaling of Bray-Curtis dissimilarities, and relative abundances at various taxonomic levels were analyzed with the R packages Phyloseq (1.26.1) and Vegan (2.5-5). Mann-Whitney test was performed to test the significance of the difference in the relative abundance of fungal genera between the two groups with “Benjamini-Hochberg” (BH) correction. Analyses were performed in R (v3.5.2).

### Isolation of Cells From Intestinal Lamina Propria of Mice

Colonic lamina propria cells (cLP) were isolated as previously described¹⁵. Briefly, colons were excised, opened longitudinally, washed of fecal contents and then cut into 1 cm pieces. Intestinal pieces were transferred into Hank’s Balanced Salt Solution (HBSS) medium (Thermo Fisher Scientific), supplemented with 2 mM EDTA, and were shaken for 8 min at 37° C. The remaining tissue was washed, minced and subsequently incubated in digestion medium consisting of RPMI 1640 (Thermo Fisher Scientific), 5% FBS, 0.5 mg/ml collagenase type VIII (Sigma-Aldrich), 5 U/ml DNase (Sigma-Aldrich), 100 IU/ml penicillin and 100 µg/m1 streptomycin (Thermo Fisher Scientific), for 40 min at 37° C. by gentle shaking at a speed of 150 rpm. The cell suspensions were filtered through a 100 µm mesh and centrifuged at 1700 rpm. The obtained cells were filtered through a 70 µm filter, washed twice with PBS and used as cLP cells.

### Flow Cytometry

The staining antibodies for flow cytometry were purchased from Thermo Fisher Scientific, Biolegend or BD Biosciences. Dead cells were excluded with eBioscience Fixable Viability Dye eFluor 506 (Thermo Fisher Scientific) during surface staining. For cell surface staining, cells were incubated with antibodies at 4° C. for 20 min. Fluorophore-conjugated antibodies against mouse antigens: CD16/CD32 (92), CD45 (30-F11), I-A/I-E (M5/114.15.2), CD11b (M1/70), Ly6G (1A8-Ly6g), CD11c (N418), CD4 (RM4-5), TCRβ (H57-597), Ly6C (HK1.4). The FoxP3 Fix/Perm kit (Thermo Fisher Scientific) was used for intracellular transcription factor staining in accordance with the manufacturer’s instructions. For cytokine staining, cells were re-stimulated with 20 nM phorbol 12-myristate 13- acetate (Sigma-Aldrich), 1.3 uM ionomycin (Sigma-Aldrich) and brefeldin A Solution (Thermo Fisher Scientific) in RPMI-1640 medium (Corning) supplemented with 10% FBS and penicillin/streptomycin for 4 hours. Intracellular staining for indicated cytokines was fixed with a Cytofix/Cytoperm kit (BD Biosciences) according to manufacturer’s instructions. The following antibodies for intracellular staining were used: FOXP3 (FJK-16S), IL-17A (eBio 17B7), IFNγ (XMG1.2), RORγt (B2D), and IL-17F (9D3.1C8). Flow cytometry was performed using a BD LSRFortessa cell analyzer (BD Biosciences) and data was analyzed with FlowJo software (TreeStar).

### In Vitro Cell-Damaging and Cell Stimulation Assays

Caco2 cells were seeded into were seeded in 24-well tissue culture treated plates at a concentration of 0.2 × 10⁵/ml in DMEM Medium (10% fetal bovine serum, 1% penicillin/streptomycin solution, these reagents were purchased from Corning or gibco) for 3 days at 37° C. Then Caco2 were co-incubated with live C. albicans at MOI 1 or 5 in DMEM Medium (serum free, 1% penicillin/streptomycin solution) for 24 hours. Supernatants were obtained for lactate dehydrogenase (CytoTox 96® Non-Radioactive Cytotoxicity Assay kit, Promega) and various cytokine measurements. RNA was extracted from C. albicans co-infected with Caco2 cells after 6-7 hours using the YeaStar RNA kit. RT-PCR was performed with C. albicans ACTIN as the control. Plasmid, primers and repair templates used in this study listed in the Supplementary Table A.

TABLE A

Supplemental:

| Plasmids used in this study | Description |
|---|---|
| pV1524 | C. albicans Solo CRISPR vector flanked by Neut5+FRT targeting regions; FLP expression driven by MAL2 promoter |
| pVXL21 | pV1524 + sgCaECE1 |
| pVXL22 | pV1524 + sgCaEFG1 |
| Oligonucleotide sequences used in this study | |
| sgRNA cloning Primers | |
| CaEfglgRNA -fwd | atttgTCACAACAGCCACCACTACCg (SEQ ID NO: 1) |
| CaEfglgRNA -rev | aaaacGGTAGTGGTGGCTGTTGTGAc (SEQ ID NO: 2) |
| CaEcelgRNA -fwd | atttgAATTTCTGGCAATCTGACGAg (SEQ ID NO: 3) |
| CaEcelgRNA -rev | aaaacTCGTCAGATTGCCAGAAATTc (SEQ ID NO: 4) |
| Repair Templates for mutagenesis | |
| CaEfg1-delta-fwd | AACGAATTAAGATTTGTTCTATTTGACTACCAAGAATATAACCCATATTATAAATATCAT (SEQ ID NO: 5) |
| CaEfg1-delta-rev | TTTGGAATTTATGGCAGAAAGCAGAAGGTGATGTACACAAATGATATTTATAATATGGGT (SEQ ID NO: 6) |
| CaEce1-delta-fwd | AACAAACAACTTTCCTTTATTTTACTACCAACTATTTTCCATTCGTTAAAATGCTCAGCA (SEQ ID NO: 7) |
| CaEcel-delta-rev | TGGAATAAAAGATTAAGCTTGTGGAAAACAAATTTTTATCTGCTGAGCATTTTAACGAAT (SEQ ID NO: 8) |
| Colony PCR/Sequencing Primers | |
| Plasmid Sequencing F Primer | Ggcatagctgaaacttcggccc (SEQ ID NO: 9) |
| CaEce1-PCR-fwd | CAATTCAAAACGAATTGCACC (SEQ ID NO: 10) |
| CaEcel-PCR-rev | ATATCAATACAGCGAGCCAT (SEQ ID NO: 11) |
| CaEfg1-PCR-fwd | AATTCATTACCAGGCGTGTT (SEQ ID NO: 12) |
| CaEfg1-PCR-rev | CGTTCATGTCAATGGATTTG (SEQ ID NO: 13) |
| Primers for RT-PCR | |
| CaEce1-PCR-fwd | cttcaaagactcccacaactcat (SEQ ID NO: 14) |
| CaEce1-PCR-rev | agcttttccgaaatattcttcaatca (SEQ ID NO: 15) |
| CaEfg1-PCR-fwd | CATTGATCCGATTTTCACAACG (SEQ ID NO: 16) |
| CaEfg1-PCR-rev | CCTACAACAGATATCAGTATCC (SEQ ID NO: 17) |
| CaActin-PCR-fwd | TCAGACCAGCTGATTTAGGTTTG (SEQ ID NO: 18) |
| CaActin-PCR-rev | GTGAACAATGGATGGACCAG (SEQ ID NO: 19) |

Murine bone marrow cells were harvested and seeded in 150 mm non-tissue culture treated plates at a concentration of 2× 10⁶/ml in RPMI Medium (10% fetal bovine serum, 10 mM HEPES solution, 1% L-glutamine, 1 mM Sodium Pyruvate, 1% MEM non-essential amino acids, 55 µM β-mercaptoethanol, 1% penicillin/streptomycin solution, these reagents were purchased from Corning or Gibco) supplemented with 50 ng/ml macrophage colony stimulating factor (M-CSF) (PeproTech) for 7 days at 37° C. On day 7, after removing the medium and washing the cells with PBS, adherent cells were incubated with cell dissociation buffer (Invitrogen) for 5 min at 37° C. mBMDMs were seeded in 96-well plates to a final number of 1 × 10⁵/well in serum-free medium for 12 hours. Then mBMDMs were co-incubated with C. albicans at MOI 5 for 16 hours. Human peripheral blood mononuclear cells (hPBMUCs) were isolated from healthy volunteers’ buffy coats requested from NYC blood center with Ficoll density gradient centrifugation media. Then CD14⁺ positive monocytes were selected by CD14 positive selection kit (Miltenyi Biotec). To differentiate monocytes into human monocyte-derived macrophages, 6 X10⁴ CD14⁺ monocytes were seeded in each well of a 96-well plate in RPMI 1640 media with 2 mM L-glutamine (Thermo Fisher Scientific) containing 10% heat-inactivated fetal bovine serum (FBS; Bio&SELL) (RPMI + FBS) and 50 ng/mL recombinant human M-CSF (ImmunoTools) and incubated for seven days at 37° C. and 5% CO₂. Medium was changed with serum-free medium overnight, and cells were stimulated with LPS (50 ng/mL). In both human and murine in vitro assays, live C. albicans (MOI=5) was added to each well, cultures were incubated for 16 hours and supernatants were obtained for lactate dehydrogenase (CytoTox 96® Non-Radioactive Cytotoxicity Assay kit, Promega) and various cytokine measurements. Cell-damage was calculated using the following equation: Cell-damage (%) =100* (Experimental absorbance (OD492)-Average of media-only absorbance (OD492))/Average of C. albicans SC5314 or parental C. albicans control strain absorbance (OD492). Murine and human IL-1β, TNFα and IL-6 were measured by ELISA kit (Biolegend). A larger panel of human cytokines was assessed by LEGENDplex™ Human Macrophage/Microglia Panel kit following the manufacturer’s instructions.

### Filamentation Assays

C. albicans strain was cultured in Sabouraud dextrose broth at 28° C. for 24 hours³¹. C. albicans strain was plated on Spider agar (2% Nutrient broth, 2% Mannitol, 0.4% potassium phosphate, 2.7% Bacto Agar, PH 7.2) and incubated at 37° C. for 5 days followed by filamentation assessment of the edge of wrinkled and smooth colonies with bright field microscopy.

### CRISPR- Mediated Genome Editing of the Human Gut-Derived Candida Albicans Isolates

All our plasmid construction were based on plasmid pV1524, a gift from Gerald Fink (http://n2t.net/addgene:111431; RRID:Addgene_111431)⁴⁶. In the plasmid 1524, the CRISPR/Cas9 cassette was inserted into the Neut5L site, a locus whose disruption was reported not to impact growth⁴⁶. Guide RNAs (gRNAs) and plasmids: gRNAs for specific gene were either immediately adjacent to or within 15 bp of the desired mutagenesis point. Phosphorylated and annealed guide sequence-containing primers were ligated into CIP (calf intestinal phosphatase)-treated BsmBI-digested parent vectors. Correct clones with the right gRNAs were identified by sequencing. Repair template were generated with 60-bp bases with homology to the sequences upstream or downstream from the target gene and containing 20-bp overlaps at their 3′ ends centered at the mutation point for the target gene, which consisted of an open reading frame (ORF) deletion of the target gene. Primers were extended to generated repair templates by thermocycling performed with Ex Taq (TaKaRa). Plasmids and Oligonucleotide sequences used in this study are listed in Supplementary Table A.

Transformation: For clinical C. albicans isolates, we achieved efficient transformation with a hybrid lithium acetate/electroporation protocol was described previously⁴⁶. 5 mL gut-derived C. albcians isolates were grown in YPD medium overnight at 30° C. in an incubator with a 150-rpm shaking speed to achieve saturation. C. albicans cells were quickly pelleted by centrifugation and resuspended in 2 ml of freshly prepared TE/lithium acetate solution (100 mM lithium acetate-10 mM Tris (pH 8.0)-1 mM EDTA (pH 8.0)) containing 50 µl 1 M dithiothreitol (DTT). C. albicans cells were incubated for 6 hours on a 30° C. roller drum. The cells were then washed twice in ice-cold sterile water, once in ice-cold 1 M sorbitol, and then resuspended in 500 µL of ice-cold 1 M sorbitol. 40 ul C. albicans cells were mixed with 5 µg digested plasmid DNA and with 6 ug purified repair templates in a pre-chilled electroporation cuvette (#9140-2002, USA scientific) and kept on ice for 5-10 minutes before electroporation. C. albicans cells was electroporated on a Bio-Rad Gene pulser with a capacitance of 25 µF and resistance of 200 Ω at 1.8 kV. The cuvette was immediately filled with 600 mL cold YPD medium, and the cell mixture was incubated for 12 hours at 30° C. before plating onto YPD selective media (1% Difco Bacto yeast extract, 2% Difco Bacto peptone, 2% glucose 200 µg nourseothricin) and selected with nourseothricin (Nat) at 200 µg/ml. In 2-3 days, colonies appeared. Colonies were picked up and re-plated with a selective YPD agar plate. Flipout of the Natr gene from C. albicans vectors was induced by overnight growth in YP maltose medium (1% Difco Bacto yeast extract, 2% Difco Bacto peptone, 2% maltose). Episomal plasmid loss experiments were performed by overnight growth in nonselective liquid YPD medium. Drug-sensitive isolates, which had either flipped out the cassette or lost the plasmid, were identified by plating for single colonies on nonselective media and subsequent identification by replica plating to selective media. Colony PCR flanking the deleted region was used to select CRISPR/Cas9-mutagenized ECE1 or EFG1 mutants, which were then confirmed using sequence analysis colony PCR products. Oligonucleotide sequences for PCR/sequencing used in this study are listed in Supplementary Table A. Whole-genome sequencing of the resulting C. albicans mutants was utilized to confirm single-gene mutagenesis in the EFG1 or ECE1 genes. All strains were stored as glycerol stocks at -80° C.

### Whole Genomic Sequencing (WGS) and Analysis

Each C. albicans isolate was cultured in SDB for 24 hours. Genomic C. albicans DNA was extracted from colonies using the QiaAmp DNA Mini Kit (Qiagen). Quality control, library preparation and deep sequencing on Illumina HiSeqX platform were performed at Novogene Co., Ltd. Raw sequencing data for each isolate was obtained. For comparison, raw sequencing data for representative C. albicans strains belonging to 17 previously established SNP-based clades⁵¹ were downloaded from the NCBI Sequence Read Archive (BioProject ID PRJNA432884). Ten strains from each SNP-based clade were selected (or when fewer than 10 strains were available, all available strains were selected) for comparison with our human gut C. albicans isolates. Both downloaded and newly sequenced WGS raw sequence data were processed according with the following workflow: Raw reads were aligned using the Burrows-Wheeler Aligner (BWA)⁶⁴ to the haplotype A chromosomes of the C. albicans strain SC5314 assembly 22 obtained from the Candida Genome Database (www.candidagenome.org). Aligned reads were processed using the Genome Analysis Toolkit version 4 (GATK4)⁶⁵. Specifically, duplicate reads were marked using the GATK4 MarkDuplicates command and variants were called using the HaplotypeCaller command. Variants were then filtered using the VariantFiltration command with filters “QD < 2.0”, “ReadPosRankSum < -8.0”, “FS > 60.0”, “MQRankSum < -12.5”, and “MQ < 40.0”. Further analysis included only variants marked “PASS” by VariantFiltration. The biallelic SNPs from the resulting VCF files were used to compute a sample-sample distance matrix and dendrogram using the SNPRelate R package⁶⁶. The resulting dendrogram was plotted using the circlize R package⁶⁷. Similar dendrograms were generated for genomic subregions using region-specific VCF files extracted using the GATK4 command SelectVariants. SNP-based clade assignments for the SRA-downloaded samples were obtained from Ropars et al.⁵¹. Clade labels in the dendrogram were propagated to newly sequenced isolates according to the following rule: if the latest common ancestor of all strains labeled clade X does not have descendants with other labels, then all its descendants receive the label for clade X. Individual VCF files were extracted from the joint VCF file using GATK4 SelectVariants. Heterozygous SNPs were extracted using bcftools⁶⁸ and heterozygous SNPs were counted in each 10 kbp of each genome using bedtools⁶⁹. Genome assemblies were generated from raw FASTQ files using Spades⁷⁰ version 3.10 with options -k 21,33,55,77 -careful. Nucleotide sequences of interest (SK1, ECE1, EFG1, CPH1, UME6, FLO8) in the genome assembly graphs (fastg files) were extracted using the querypaths command of Bandage⁷¹. The corresponding amino acid sequences were generated with the transeq command of EMBOSS² using translation table 12 (Alternative Yeast Nuclear).

### Fluorescence In Situ Hybridization (FISH)

8-10-week-old GF mice were orally gavaged with C. albicans (1 × 10⁸ CFU/mouse) at day 1 and day 2. After fungal colonization, mice were maintained within sterile vinyl isolators for 21 days and then sacrificed at day 23. Colon sections were collected along with feces and fixed in a methacarn solution (60% methanol, 30% chloroform, and 10% glacial acetic acid) at room temperature (RT) overnight. The following procedures were used to wash the colon sections: 2 ×30 minutes in methanol, 2 × 20 minutes in ethanol, 2 × 20 minutes in xylene substitute. All chemical reagents are purchased from Sigma-Aldrich. Colon sections were then immersed for two hours at 70° C. in melted paraffin wax. Tissues were embedded and further cut at 10 µm by the histology facility at WCMC. The following procedures were used to dewax colon sections: 2× 10 minutes at 60° C. in xylene substitution, 2×5 minutes at RT in ethanol. For C. albicans detection, DNA hybridization was performed for three hours at 50° C. using a pan-fungal probe conjugated to Cy3 (/5Cy3/CTCTGGCTTCACCCTATTC (SEQ ID NO: 24), Integrated DNA Technologies, 0.25 µg per sample)³⁹, and mucus layers were stained with FITC conjugated UEA-1 (Sigma, L9006-1MG) for overnight incubation at 4° C. in a sealed chamber. All sections were further stained for 4 minutes at room temperature with 4′,6-diamidino-2-phenylindole (DAPI). The sections were then mounted with ProLong™ Gold Antifade Mountant (Thermo Fisher Scientific) and imaged on Zeiss LSM880. Images was merged by Fiji with the scale bar indicating 25 µm.

### Histological Staining

Distal colon sections were collected and fixed in 10% neutral buffered formalin for 24 hours at room temperature and then were transferred to 70% ethanol. Complete colon tissue was embedded in paraffin, sectioned and stained with hematoxylin and eosin by IDEXX BioAnalytics. Blinded histological evaluation was conducted by a board-certificated pathologist (C. Y.). Samples were scored according to the following criteria: area involved (0-4), erosion/ulceration (0-4), follicles (0-3), edema (0-3), fibrosis (0-3), crypt loss (0-4), granulocytes (0-3), mononuclear cells (0-3), and crypt damage/apoptosis (0-4). Scores were summed to give a total inflammation score.

### Statistical Analysis

The investigators were blinded during colonic mucosa-enriched lavage sample and data collection. The investigators were blinded for the colon histological evaluation. As noted, based on quality control, one non-IBD sample was excluded from mycobiome sequencing and analysis. No data or mice were excluded for other analysis. No statistical analysis was used to determine the appropriate sample size. n indicated in the figure legends means the number of mice or human subjects in the experiments. Data are representative of at least two independent experiments as indicated. Unless otherwise specified, P values were calculated using unpaired nonparametric Mann-Whitney test or one-way ANOVA followed by the Tukey’s post hoc test by GraphPad Prism (GraphPad Software) as indicated in the figure legends. The linear regression was performed with JMP Data analysis software.

Data availability. ITS sequencing data are available in NCBI Sequence Read Archive (SRA) under the Bioproject ID PRJNA610042 (https://dataview.ncbi.nlm.nih.gov/object/PRJNA702809?reviewer=dv6ap8eofk761vnrhjp20fbild ). The data from whole-genome sequencing of human gut-derived C. albicans isolates are available in the NCBI Sequence Read Archive (SRA) under Bioproject ID PRJNA702809 (http://www.ncbi.nlm.nih.gov/bioproject/702809).

### Example 2: candidalysin-Secreting C. albicans Strains as a Biomarker for Selecting IBD Patients for IL-1-Blocking Therapy

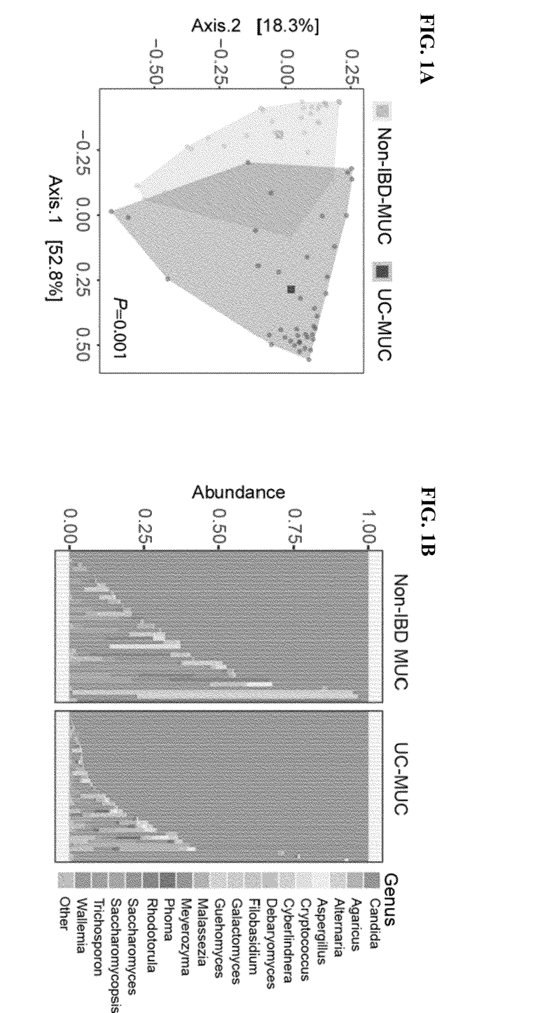

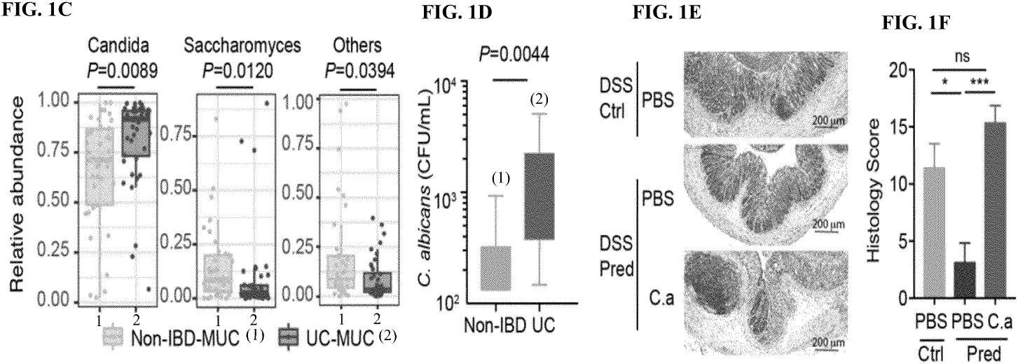

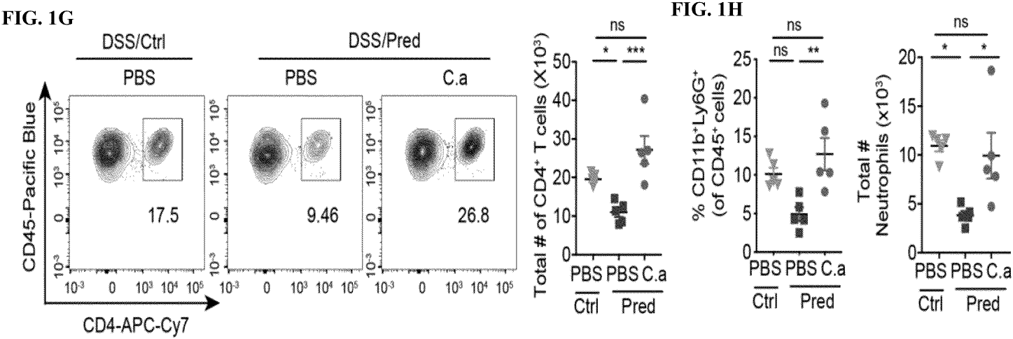

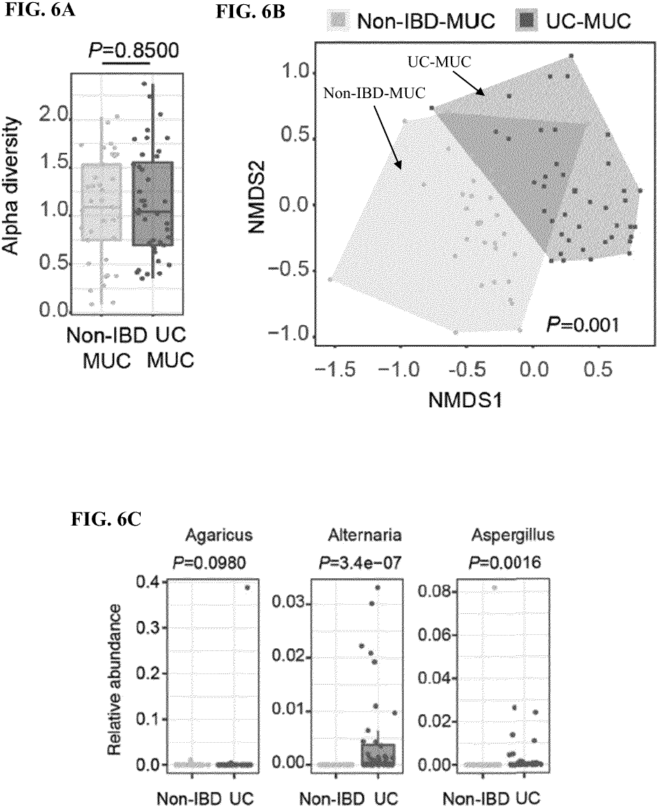

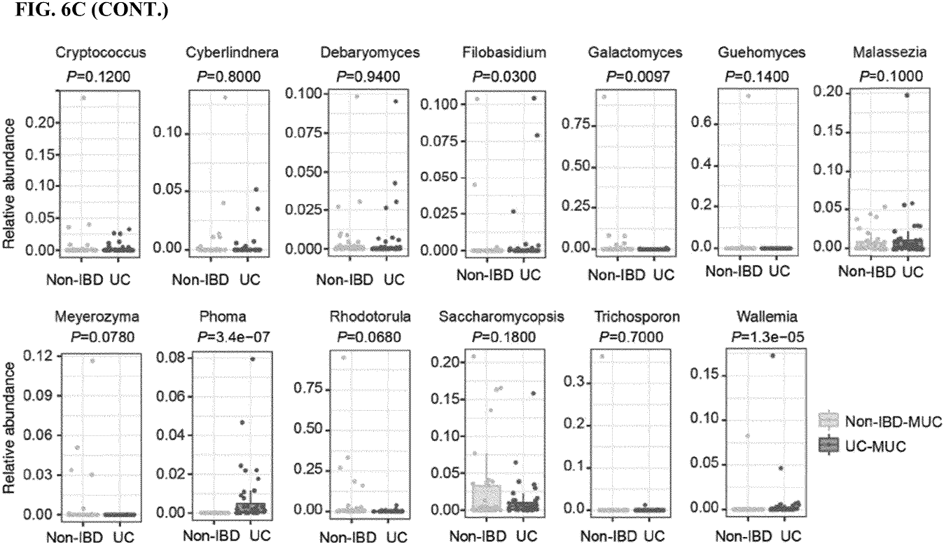

To study the functional consequences of human gut mycobiota on the host, we focused our analysis on UC which targets the colon: a site where commensal fungi are highly abundant and are known to interact with host immunity^{5,7,16,17}. To enrich for potentially immunoreactive mycobiota associated with the intestinal mucosa, we obtained colonic mucosa lavage samples from 38 non-IBD individuals and 40 patients with UC undergoing colon cleansing - a process that removes fecal and other luminal contents in preparation for colonoscopy and we performed ITS sequencing of fungal ribosomal DNA (rDNA). The fungal community composition differences analysis revealed a distinct clustering between patients with non-IBD and UC individuals, while alpha diversity remained similar within each group (FIG. 1A and FIGS. 6A-6B). Further analyses at the genus level revealed the presence of 18 highly prevalent fungal genera with > 0.2% average relative abundance across all samples (FIG. 1B). Among those, Candida and Saccharomyces represented the most abundant genera (FIG. 1B and Supplementary Table B), indicating that Candida spp. are the most common inhabitants of the human colonic mucosa.

Supplemental TABLE B

Fungal genera abundance detected in non-IBD and UC individuals

| Genus | Count non-IBD | Mean non-IBD | SD non-IBD | Count IBD | Mean IBD | SD IBD | P value |
|---|---|---|---|---|---|---|---|
| Candida | 37 | 0.65902292 1 | 0.27854132 7 | 40 | 0.81782781 5 | 0.22034398 | 0.0023 4 |
| Saccharomyces | 37 | 0.14782901 9 | 0.17357219 4 | 40 | 0.09426436 | 0.20164941 1 | 0.0048 6 |
| Rhodotorula | 37 | 0.05549158 3 | 0.16962011 7 | 40 | 0.00217492 9 | 0.00652406 1 | 0.0357 2 |
| Galactomyces | 37 | 0.03187558 6 | 0.15218538 8 | 40 | 0.00033414 7 | 0.00151737 5 | 0.0030 7 |
| Saccharomycopsis | 37 | 0.02629744 1 | 0.05356443 3 | 40 | 0.01250209 5 | 0.02697903 7 | 0.1523 1 |
| Guehomyces | 37 | 0.02015809 6 | 0.12086097 8 | 40 | 1.22E-05 | 5.57E-05 | 0.1142 1 |
| Trichosporon | 37 | 0.00986924 2 | 0.05986706 8 | 40 | 0.00032686 2 | 0.00194113 3 | 0.6236 7 |
| Cryptococcus | 37 | 0.00918626 4 | 0.03975278 3 | 40 | 0.00351015 6 | 0.00773539 1 | 0.0912 8 |
| Malassezia | 37 | 0.00795752 5 | 0.01418454 | 40 | 0.01364634 | 0.03287812 4 | 0.0705 6 |
| Meyerozyma | 37 | 0.00642838 6 | 0.02152392 8 | 40 | 8.75E-06 | 5.26E-05 | 0.0454 |
| Other | 37 | 0.00577026 2 | 0.01854583 5 | 40 | 0.01134580 2 | 0.02155674 5 | 0.0043 9 |
| Cyberlindnera | 37 | 0.00572645 7 | 0.02236318 2 | 40 | 0.00261514 5 | 0.00974223 3 | 0.7571 1 |
| Debaryomyces | 37 | 0.00549093 2 | 0.01708701 | 40 | 0.0056597 | 0.01703752 9 | 0.9369 6 |
| Filobasidium | 37 | 0.00411540 8 | 0.01836292 6 | 40 | 0.00558682 3 | 0.02063140 5 | 0.0142 |
| Wallemia | 37 | 0.002237 | 0.01360714 | 40 | 0.00667870 2 | 0.02792275 3 | 2.1E-06 |
| Aspergillus | 37 | 0.00221533 | 0.01347532 7 | 40 | 0.00229488 8 | 0.00607512 2 | 0.0003 3 |
| Agaricus | 37 | 0.00032854 7 | 0.00179205 7 | 40 | 0.00990963 3 | 0.06134691 9 | 0.0618 9 |
| Phoma | 37 | 0 | 0 | 40 | 0.00686873 | 0.01512825 3 | 3.6E-08 |
| Alternaria | 37 | 0 | 0 | 40 | 0.00443286 9 | 0.00852981 2 | 3.6E-08 |

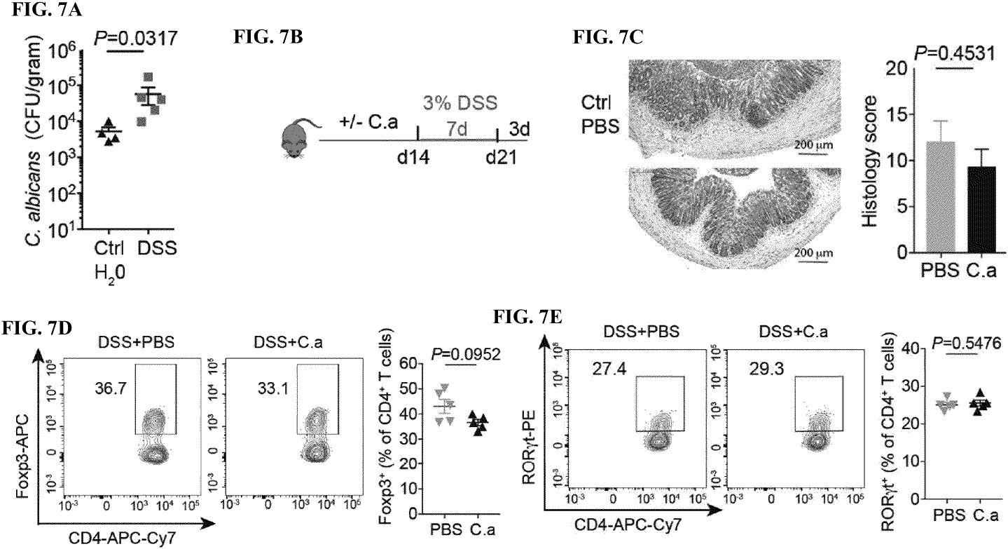

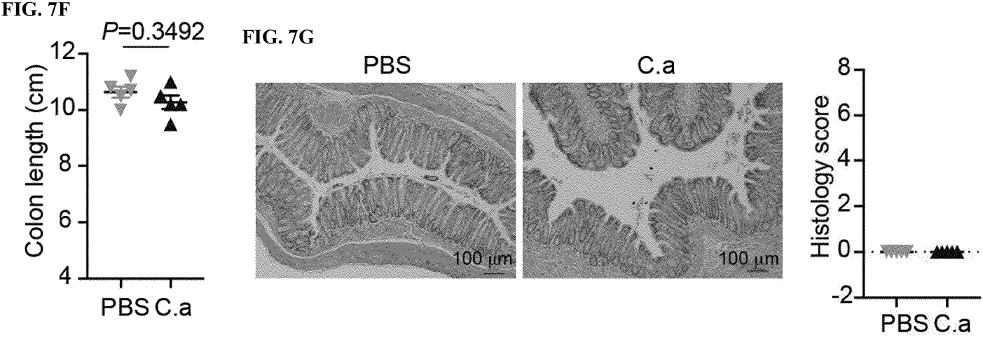

A comparison of the relative abundance of the identified genera revealed a significant increase in Candida and a pronounced reduction of Saccharomyces in the mucosa-enriched mycobiome of UC patients when compared to non-IBD individuals (FIG. 1C and FIG. 6C). In contrast, a rare abundance of food-derived Debaromyces spp. or other skin-resident fungi^{8,9} are not altered with UC disease status (FIG. 1B and FIG. 6C). Using a culture dependent assay, we confirmed the sequencing results by observing higher Candida albicans levels in the mucosa of UC patients as compared with non-IBD individuals (FIG. 1D). To experimentally determine whether increased C. albicans abundance influences intestinal inflammation, we orally gavaged wild-type (WT) C57BL/6 specific pathogen free (SPF) mice with C. albicans SC5314, a commonly used laboratory strain and determined its effect on dextran sulfate sodium (DSS)-induced model of colitis. While DSS-induced intestinal inflammation provided a niche for C. albicans colonization and overgrowth (FIG. 7A), disease severity and immune cell infiltration was not affected by the fungus in this model (FIGS. 7B-7E), findings consistent with previous studies^{8,15}. Furthermore, C. albicans did not cause spontaneous colitis following a prolonged colonization (up to 4 months, FIGS. 7F-7G). Thus, while Candida spp. are a disease contributing factor during genetic deficiencies targeting antifungal immunity pathways^{15,18}, C. albicans does not cause spontaneous intestinal inflammation in an immunocompetent host with intact antifungal immunity, albeit inflammation appeared to be a driver of C. albicans expansion in the gut.

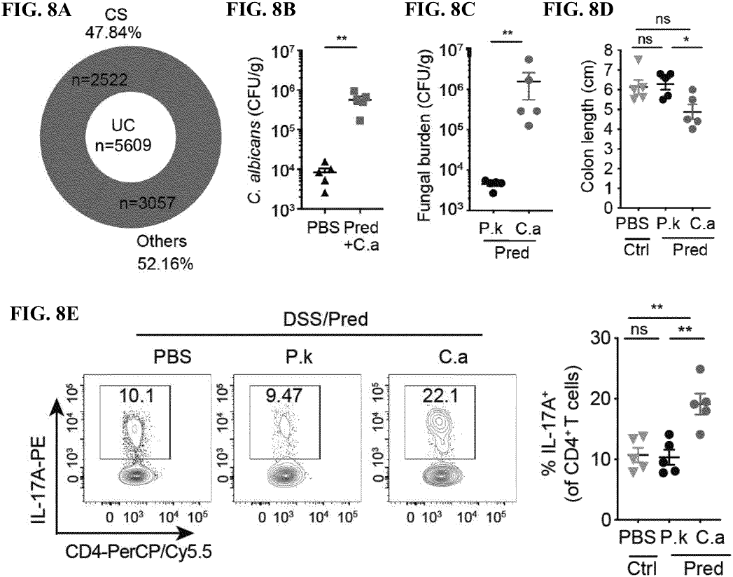

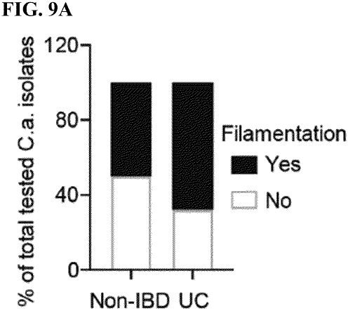

Since Candida spp. influence intestinal homeostasis in a context dependent manner with immunity-related factors being among the key drivers^{15,18,19}, we sought to experimentally determine whether C. albicans colonization influences intestinal disease during immunomodulation. Thus, we utilized a mouse model of dextran sulfate sodium (DSS)-induced inflammation under corticosteroid treatment: a mainstay remission induction therapy in UC treatment used both internationally²⁰ and in our clinics (FIG. 8A). Treatment with prednisolone, allowed the establishment of sustained intestinal fungal colonization, overcoming C. albicans colonization resistance in the murine gut²¹ (FIG. 8B). While WT SPF mice only developed a mild disease, mice colonized with C. albicans displayed enhanced histopathology, as characterized by increased mucosal erosion, crypt destruction, and inflammatory cell infiltration in the colon (FIGS. 1E-1H). Further characterization revealed increased CD4⁺ T cell and neutrophil infiltration in the colon (FIGS. 1G-1H). In contrast, colonization with Pichia kudriavzevii, which is present in the intestinal mucosa of healthy individuals⁸, did not contribute to pathogenic immune response and colitis pathology (FIGS. 8C-8E). This indicates that while not a trigger of spontaneous intestinal inflammation, commensal C. albicans expands during intestinal disease and associated therapy to negatively influence disease outcomes in this experimental setting.

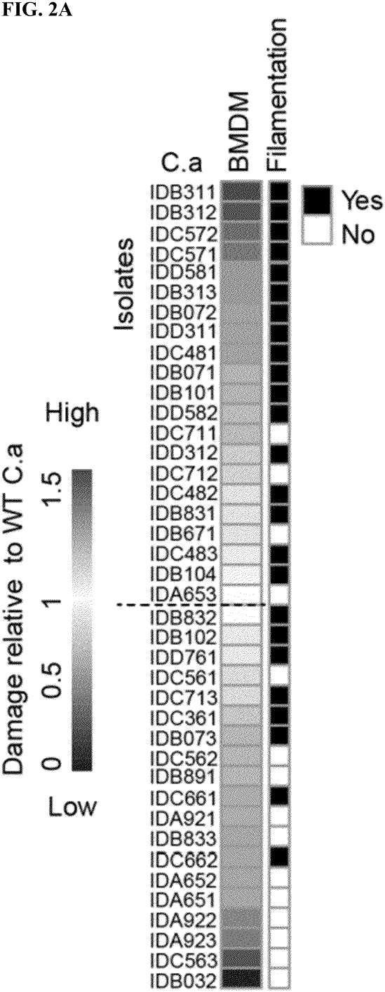

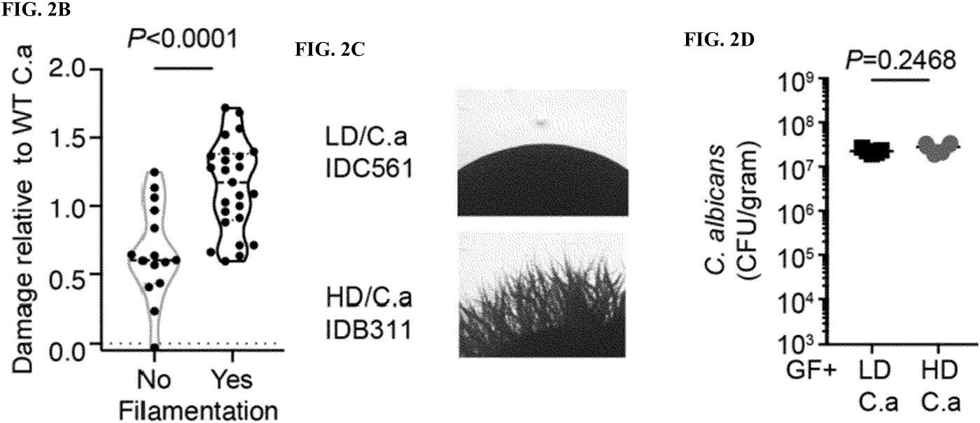

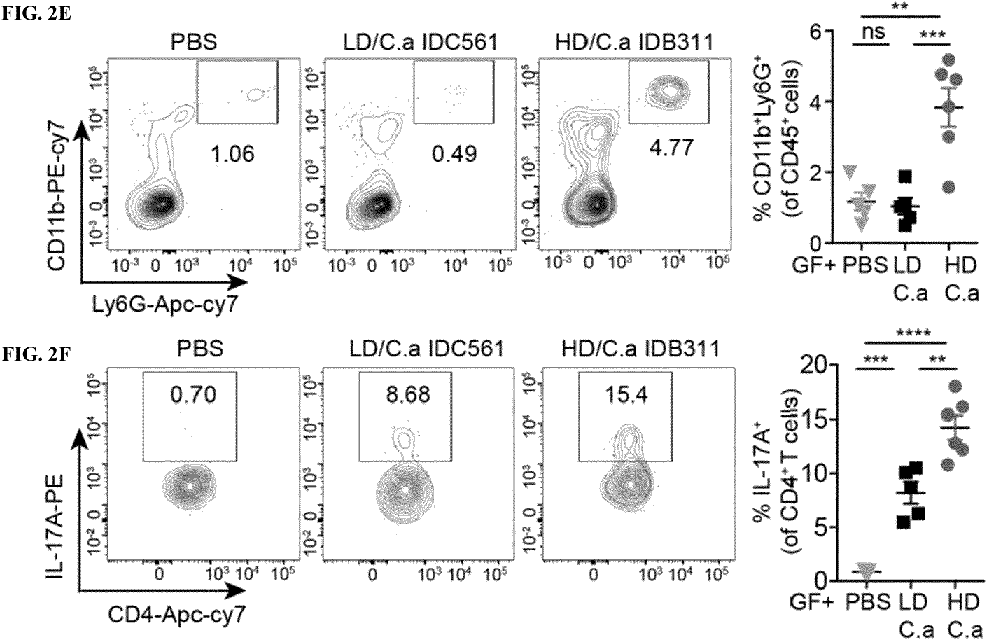

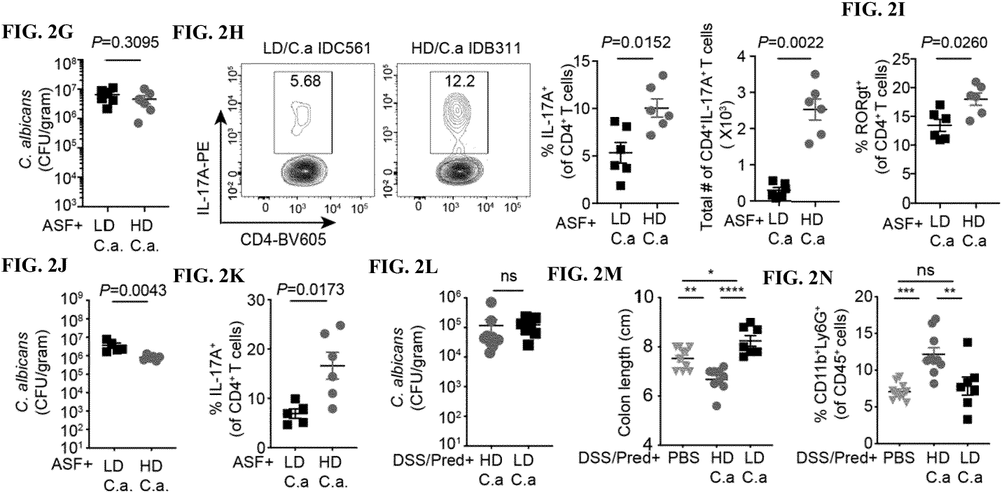

Current mechanistic studies on the role of fungi in intestinal immunity and disease are limited by the use of a handful of laboratory fungal strains of non-intestinal origin^{8,15,19,22}. In contrast, the bacterial microbiota field has adopted culturomic approaches that allow bona fide human gut-derived bacteria to be isolated and studied in the context of microbial biology and host-microbe interactions allowing for several significant discoveries in the microbiome field²³⁻²⁶. Since fungal strain-specific features can dramatically influence host immunity and infectious disease outcomes²⁷⁻³⁰, we reasoned that important insights on the role of fungi in gut inflammation could be gained from fungal strains isolated from the human intestine. The results we obtained through ITS sequencing (FIGS. 1B-1C) informed the development of culture-based methods that allowed us to detect, isolate and collect live fungi from preserved colonic mucosa-enriched lavages of non-IBDs and UC patients. Using this approach, we obtained multiple isolates of C. albicans from the colonic mucosa of UC patients (FIG. 1D). Since intestinal macrophages are key to initiation and induction of antifungal immunity in the colonic mucosa¹⁵ and virulent C. albicans strains can damage these cells³¹⁻³⁴, we next tested the macrophage-damaging capacity of specific human mucosa-associated C. albicans isolates. Notably, gut C. albicans isolates could be ranked into two major categories: a high-damaging (HD/C.a) group consisting of isolates with an increased or similar ability to inflict macrophage damage as compared with C. albicans SC5314, and low-damaging isolates with a decreased cell-damaging capacity (LD/C.a) (FIG. 2A). The hyphal morphogenesis program in C. albicans is key to virulence factor production and is a critical driver of tissue invasion, damage and pathogenesis at several mucosal surfaces³⁵⁻³⁷. Conversely, mutations in transcriptional regulators of hyphal morphogenesis can arise during a commensal lifestyle ²⁸_{′}³¹_{′}³⁸_{′}³⁹. Thus, we randomly selected three C. albicans isolates per human subject to assess phenotypic features of these human gut-derived C. albicans strains in vitro using a filamentation phenotypic assay (Supplementary Table C).

Supplemental TABLE C

Fungal strains used in the study

| | | | |
|---|---|---|---|
| Fungal Strain | Species | Strain Description | Patient |
| SC5314 (Moyes et al., 2016) | Candida albicans | Blood-derived reference strain | N/A |
| M1477 (Moyes et al., 2016) | Candida albicans | BWP17+Cp120 (Parental Strain) | N/A |
| M2047 | Candida albicans | ecelD/D (ece mutant) | N/A |
| IDA651 | Candida albicans | Gut-derived commensal strain | Ulcerative colitis |
| IDA652 | Candida albicans | Gut-derived commensal strain | Ulcerative colitis |
| IDA653 | Candida albicans | Gut-derived commensal strain | Ulcerative colitis |
| IDA921 | Candida albicans | Gut-derived commensal strain | Ulcerative colitis |
| IDA922 | Candida albicans | Gut-derived commensal strain | Ulcerative colitis |
| IDA923 | Candida albicans | Gut-derived commensal strain | Ulcerative colitis |
| IDB311 | Candida albicans | Gut-derived commensal strain | Ulcerative colitis |
| IDB312 | Candida albicans | Gut-derived commensal strain | Ulcerative colitis |
| IDB313 | Candida albicans | Gut-derived commensal strain | Ulcerative colitis |
| IDB831 | Candida albicans | Gut-derived commensal strain | Ulcerative colitis |
| IDB832 | Candida albicans | Gut-derived commensal strain | Ulcerative colitis |
| IDB833 | Candida albicans | Gut-derived commensal strain | Ulcerative colitis |
| IDB071 | Candida albicans | Gut-derived commensal strain | Ulcerative colitis |
| IDB072 | Candida albicans | Gut-derived commensal strain | Ulcerative colitis |
| IDB073 | Candida albicans | Gut-derived commensal strain | Ulcerative colitis |
| IDB101 | Candida albicans | Gut-derived commensal strain | Ulcerative colitis |
| IDB102 | Candida albicans | Gut-derived commensal strain | Ulcerative colitis |
| IDB104 | Candida albicans | Gut-derived commensal strain | Ulcerative colitis |
| IDC481 | Candida albicans | Gut-derived commensal strain | Ulcerative colitis |
| IDC482 | Candida albicans | Gut-derived commensal strain | Ulcerative colitis |
| IDC483 | Candida albicans | Gut-derived commensal strain | Ulcerative colitis |
| IDC571 | Candida albicans | Gut-derived commensal strain | Ulcerative colitis |
| IDC572 | Candida albicans | Gut-derived commensal strain | Ulcerative colitis |
| IDC661 | Candida albicans | Gut-derived commensal strain | Ulcerative colitis |
| IDC662 | Candida albicans | Gut-derived commensal strain | Ulcerative colitis |
| IDC711 | Candida albicans | Gut-derived commensal strain | Ulcerative colitis |
| IDC712 | Candida albicans | Gut-derived commensal strain | Ulcerative colitis |
| IDB032 | Candida albicans | Gut-derived commensal strain | Non-IBD |
| IDB671 | Candida albicans | Gut-derived commensal strain | Non-IBD |
| IDB891 | Candida albicans | Gut-derived commensal strain | Non-IBD |
| IDC361 | Candida albicans | Gut-derived commensal strain | Non-IBD |
| IDC561 | Candida albicans | Gut-derived commensal strain | Non-IBD |
| IDC562 | Candida albicans | Gut-derived commensal strain | Non-IBD |
| IDC563 | Candida albicans | Gut-derived commensal strain | Non-IBD |
| IDD311 | Candida albicans | Gut-derived commensal strain | Non-IBD |
| IDD312 | Candida albicans | Gut-derived commensal strain | Non-IBD |
| IDD581 | Candida albicans | Gut-derived commensal strain | Non-IBD |
| IDD582 | Candida albicans | Gut-derived commensal strain | Non-IBD |
| IDD761 | Candida albicans | Gut-derived commensal strain | Non-IBD |
| IDB311 (ecelD/D) | Candida albicans (ECE1 mutant) | Genetically modified from IDB311 strain | |
| IDC561 (ecelD/D) | Candida albicans (ECE1 mutant) | Genetically modified from IDC561 strain | |
| IDB101 (ecelD/D) | Candida albicans (ECE1 mutant) | Genetically modified from IDB101strain | |
| IDB891 (ecelD/D) | Candida albicans (ECE1 mutant) | Genetically modified from IDB891 strain | |
| IDB311 (efg1D/D) | Candida albicans (EFGmutant) | Genetically modified from IDB311 strain | |
| IDB101 (efg1D/D) | Candida albicans (EFG1 mutant) | Genetically modified from IDB101strain | |

All strains are derived from this study unless indicated otherwise

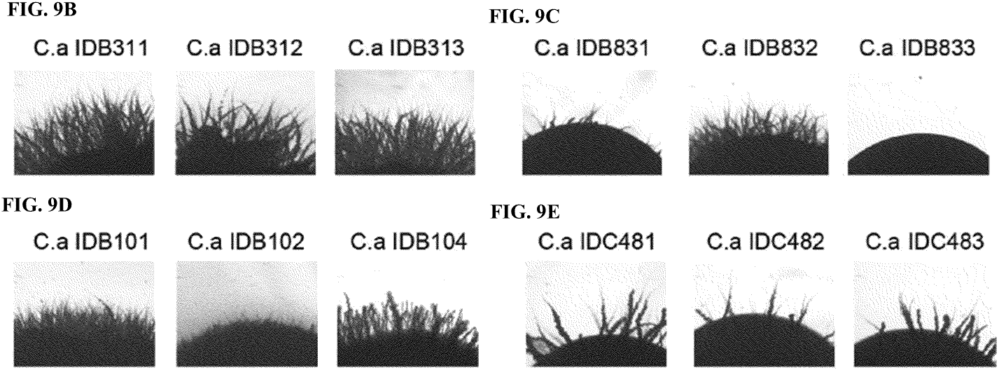

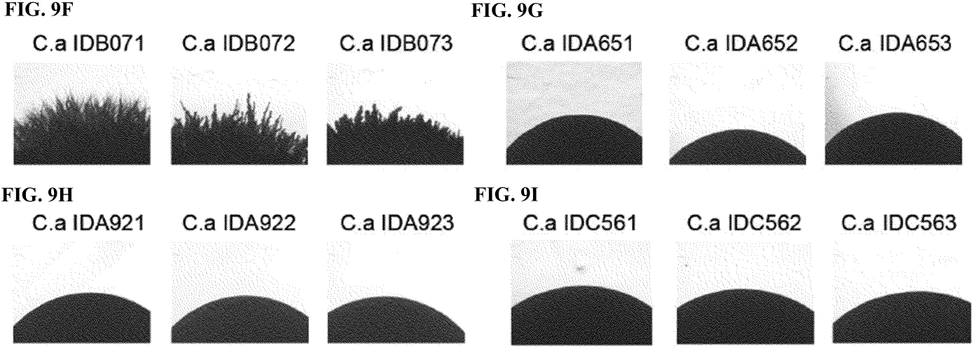

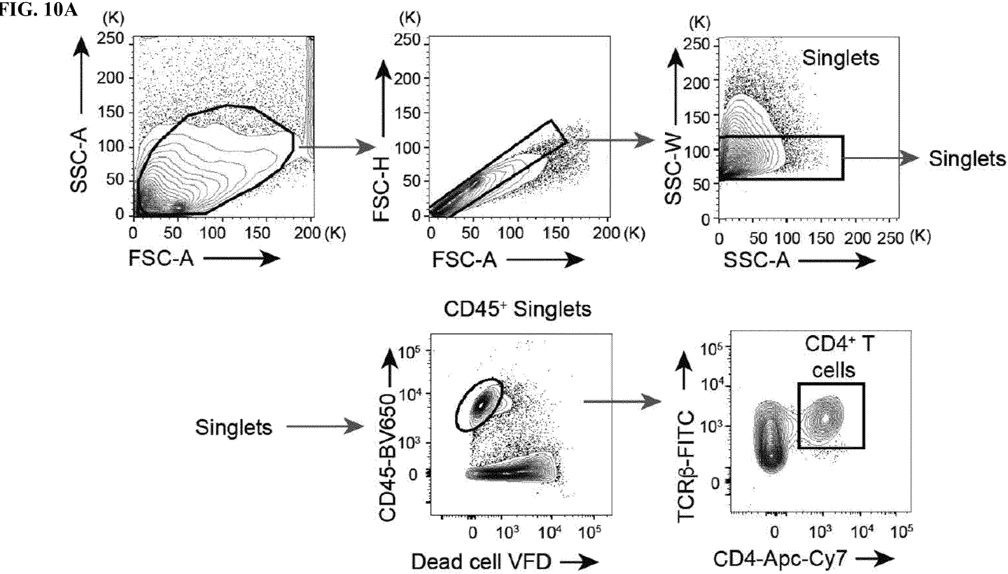

Contrary to the prediction that naturally gut-adapted C. albicans strains would be poorly filamentous in response to a filament-inducing stimulus³¹, a considerable number (~60%) (FIG. 9A) of the isolates formed filaments regardless of the host disease status (FIG. 2A and FIGS. 9B-9I). Furthermore, some C. albicans isolates originating from the same host showed slightly different filamentation phenotypes (FIGS. 9C-9D). Notably, C. albicans isolates that responded to filament-inducing stimuli induced significantly more damage of bone marrow-derived macrophages (mBMDM) compared to isolates unable to form filaments (FIGS. 2A-2B). This demonstrates that the cell-damaging capacity of gut C. albicans cells is linked to filamentation ability.

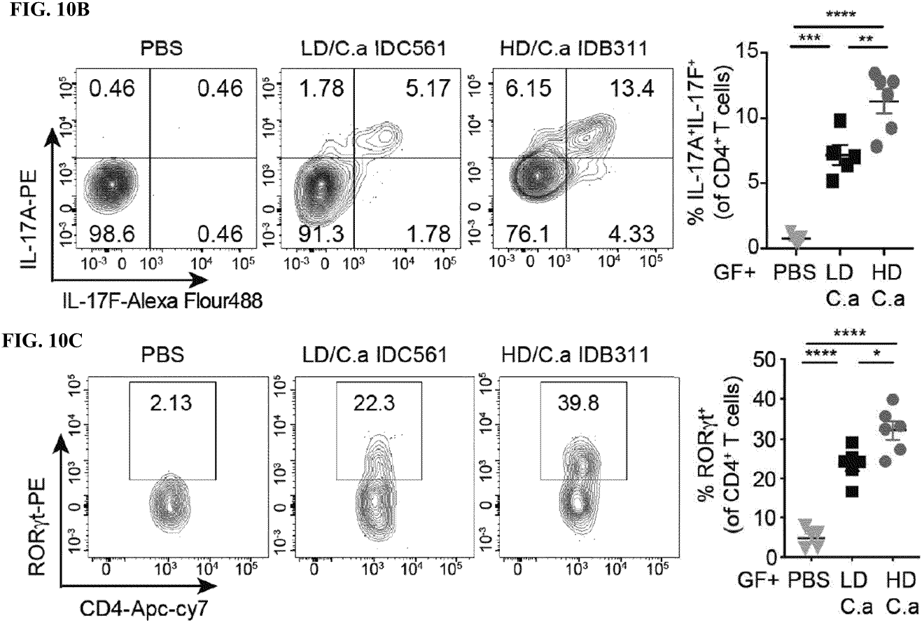

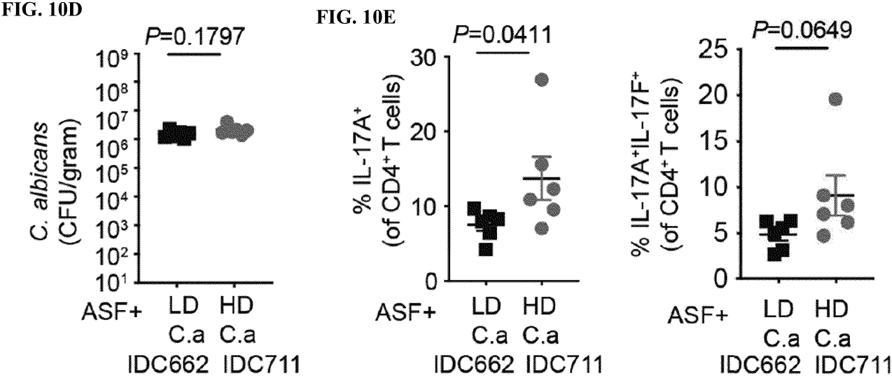

Given these findings, we next assessed immune responses to HD/C.a and LD/C.a isolates (FIG. 2C) in vivo. To explore the direct effect of these isolates on the host, we first used germ-free (GF) mice. Upon colonization with a HD/C.a strain (HD/C.a IDB311), we observed robust proinflammatory responses including neutrophil infiltration and Th17 responses in the colon, while colonization with a LD/C.a strain (LD/C.a IDC561) induced poorer Th17 responses and neutrophil infiltration (FIGS. 2E-2F and FIGS. 10A-10C). The effect was independent of fungal load as each strain stably colonized the murine gut at similar levels for the duration of the experiment (FIG. 2D).

Fungi and bacteria co-exist in a complex web of interactions in the gut where bacteria can alter C. albicans colonization in mice^{21,40}. To avoid the influence on host immunity by gut-indigenous bacteria and/or fungi present in different colonies of SPF mice^{41,42}, we utilized altered Schaedler flora (ASF)-colonized mice that are mycobiota-free, and carry a defined community of 8 functionally diverse bacteria to assess host immunity to specific C. albicans isolates^{40,43,44.} HD/C.a IDB311 and LD/C.a IDC561 gut isolates stably colonized ASF mice at a similar level (FIG. 2G).

However, only the HD/C.a isolate induced strong proinflammatory immune response (FIGS. 2H-2I). The strong immune responses induced by high-damaging isolates were further confirmed upon intestinal colonization with several HD/C.a (IDB101, IDC711) or LD/C.a (IDB891, IDC662) (FIGS. 2J-2K and FIGS. 10D-10E). Consistent with their ability to induce strong proinflammatory immunity in vivo, HD/C.a (IDB311) contributed to severe intestinal inflammation in murine model of DSS induced-colitis under prednisolone therapy when compared to LD/C.a (IDC561) (FIGS. 2L-2N). Thus, the striking capacity of specific human-gut derived C. albicans isolates to induce proinflammatory immunity is strain-dependent and correlates directly with their ability to inflict cell damage in a manner resilient to the influence of intestinal bacteria.

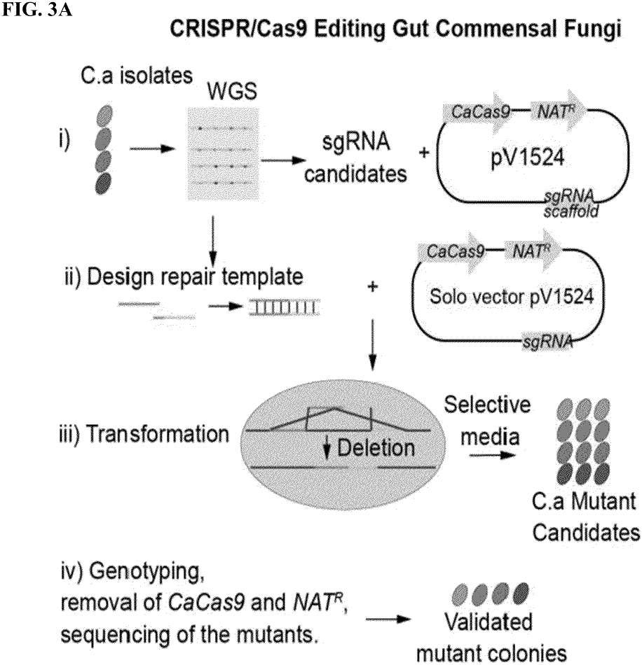

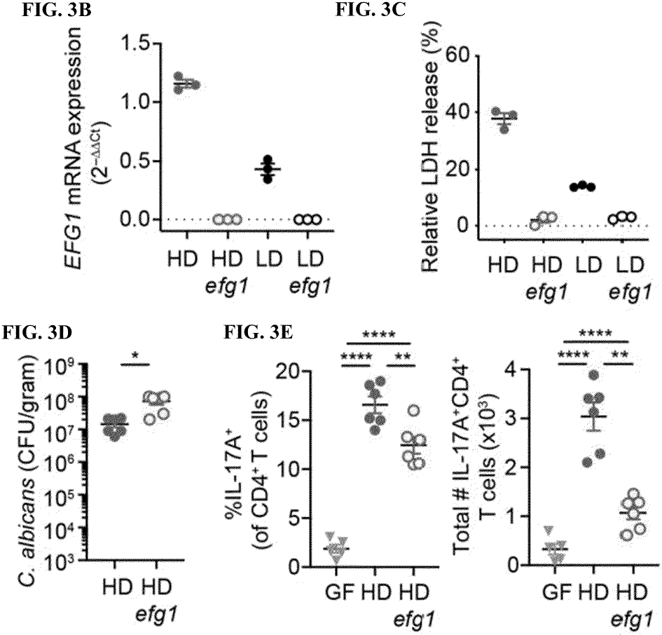

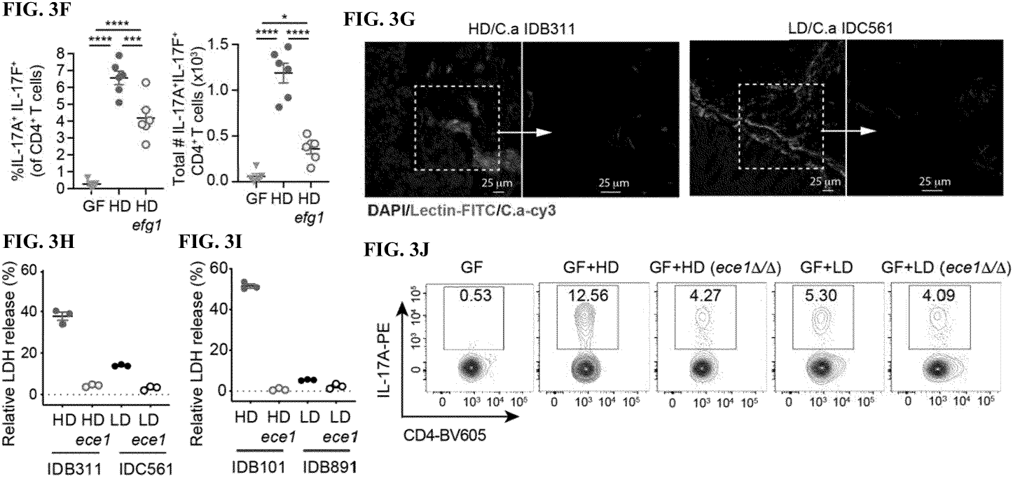

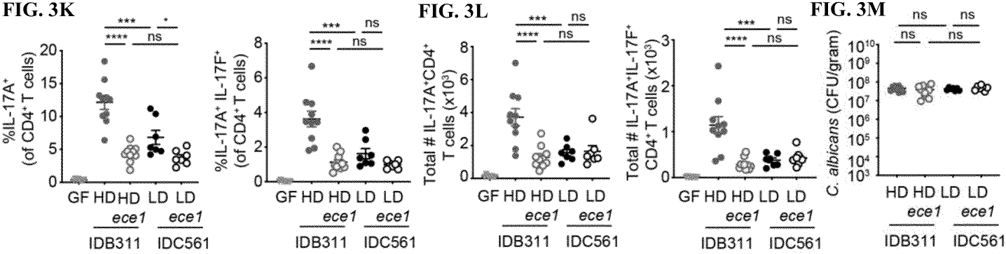

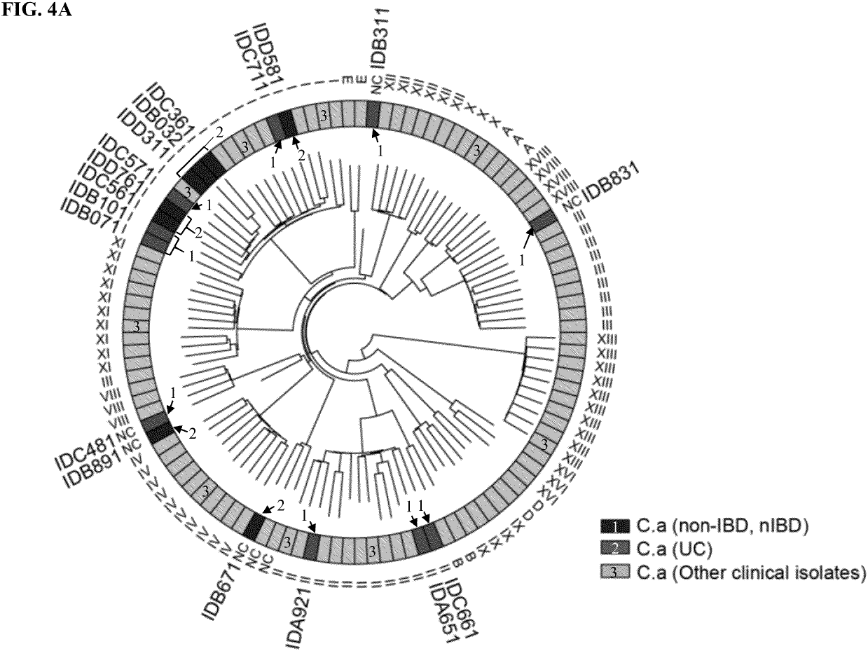

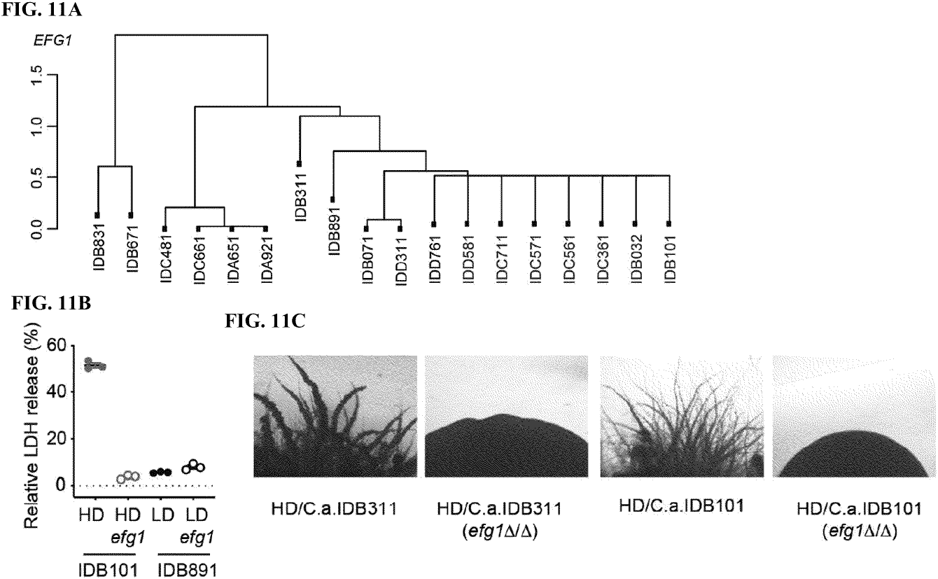

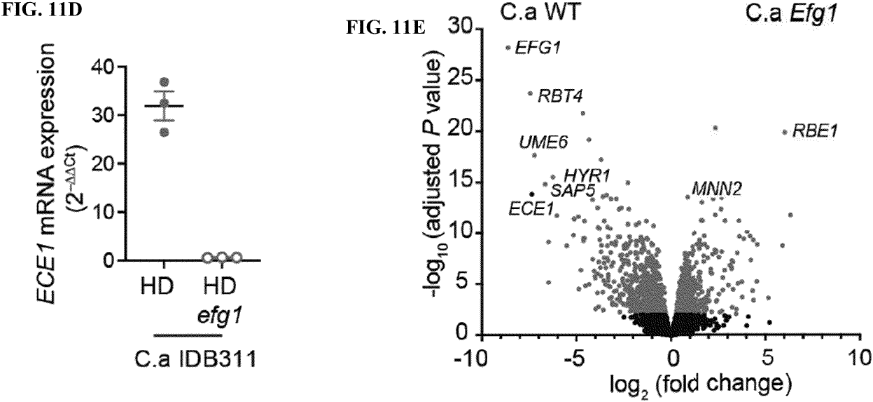

Having observed a strong correlation between filamentation, cell damage and intestinal immunity activation, we reasoned that transcriptional regulation of C. albicans morphogenesis program might play a key role in the observed phenotypes. In addition to its role in the yeast-to-hyphal transition during infection, the transcription factor enhanced filamentous growth protein 1 (EFG1) and related pathways have also been linked to the C. albicans in vivo commensalism program in the murine gut^{39,45}. Initial assessment of human gut C. albicans strains revealed a dramatic decrease in EFG1 expression in LD compared to HD strains that was unrelated to specific genetic variation in this gene or other genes related to filamentation (FIG. 3B, FIG. 11A and FIGS. 7), suggesting a role for this transcription factor in the HD phenotypes. To investigate the functional role of EFG1 among clinical isolates, we adopted a CRISPR-based technique for gene targeting in Candida spp. This system ⁴⁶_{′}⁴⁷ consists of a Candida-compatible Cas9 nuclease in a single vector and an installable synthetic guide RNA (sgRNA) that directs Cas9-mediated cleavage in the target regions of our C. albicans isolates and uses a marker free short repair template (100 base pairs) for homology directed repair (HDR) (FIG. 3A). The approach allows for selective gene targeting (by achieving homozygous recessive mutations in genes of interest) across multiple gut isolates of C. albicans (FIG. 3A) circumventing substantial limitations posed by genetic and chromosome variations among strains⁴⁸. Notably, EFG1 ablation in HD strains abrogated their ability to cause cell damage and led to a significant decrease of Th17 cell accumulation in the colon (FIGS. 3B-3F and FIG. 11B). The observed strain dependent immune phenotype was independent of intestinal expansion (although ablation of EFG1 led to intestinal overgrowth of HD/C.a, FIG. 3D) or filamentation capacity in vivo (LD strains with lower expression of EFG1 that lack filamentation in vitro, formed both hyphal and yeast cells in the gut colonic mucosa in vivo FIG. 3G and FIG. 13). Furthermore, no direct evidence of hyphal C. albicans adhesion/penetration into the intestinal epithelium was observed (FIG. 3G and FIG. 13), while mixed yeast and hyphae C. albicans morphotypes for both HD and LD strains were still present in the adjacent mucosa. Together, these data suggest that a specific fungal factor rather than hyphal morphology per se was the cause of the observed immune activation phenotypes in the gut.

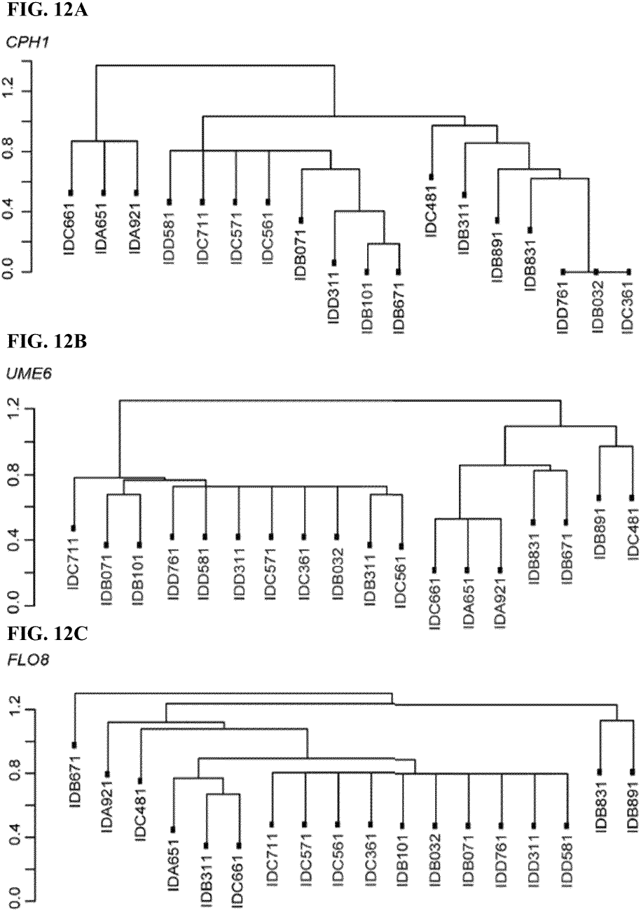

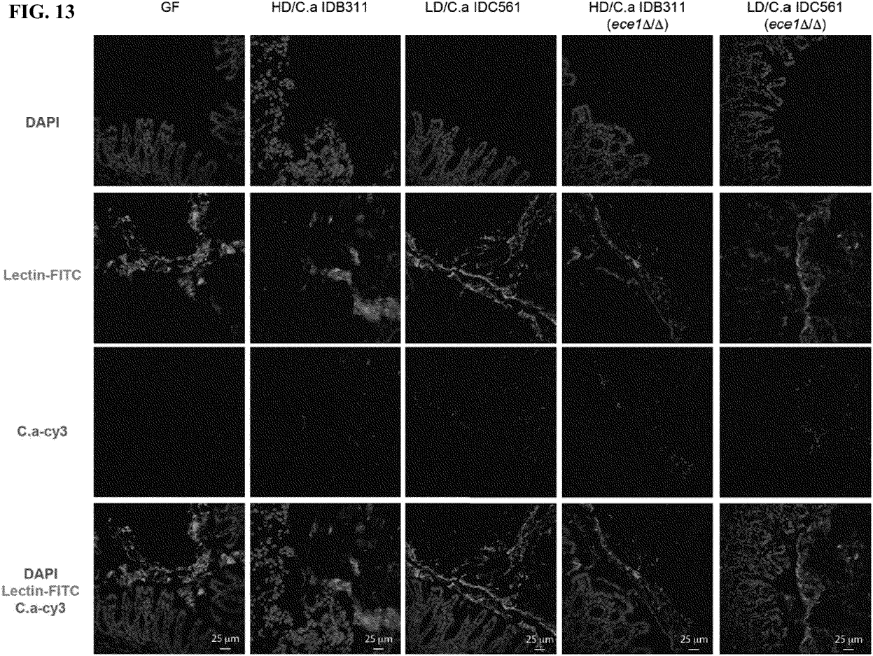

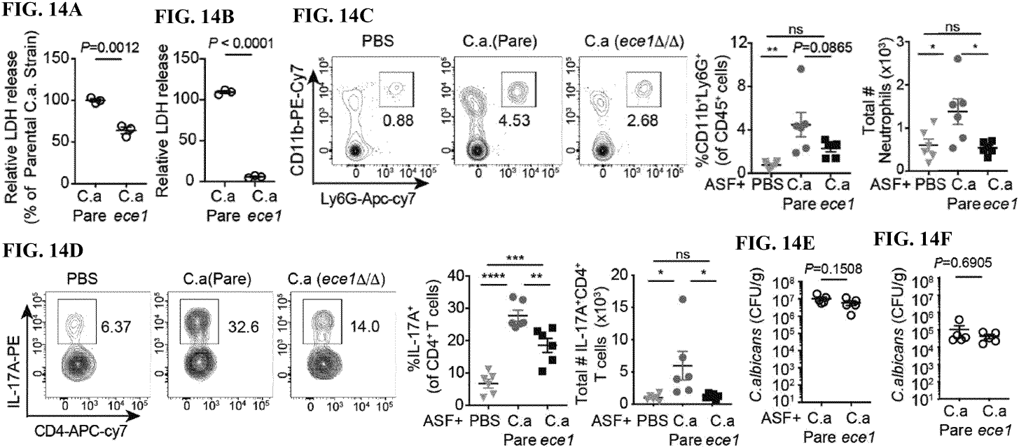

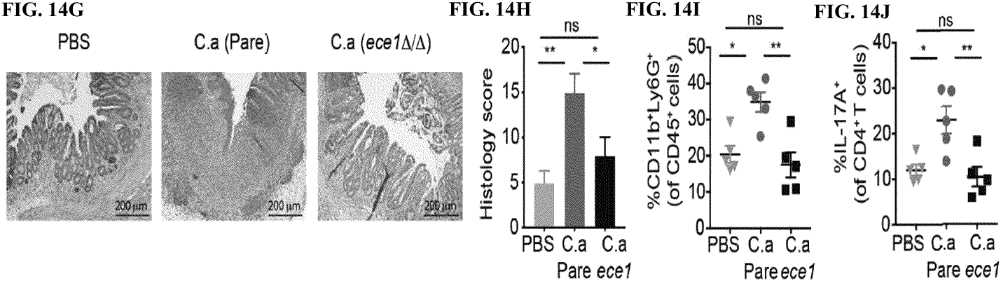

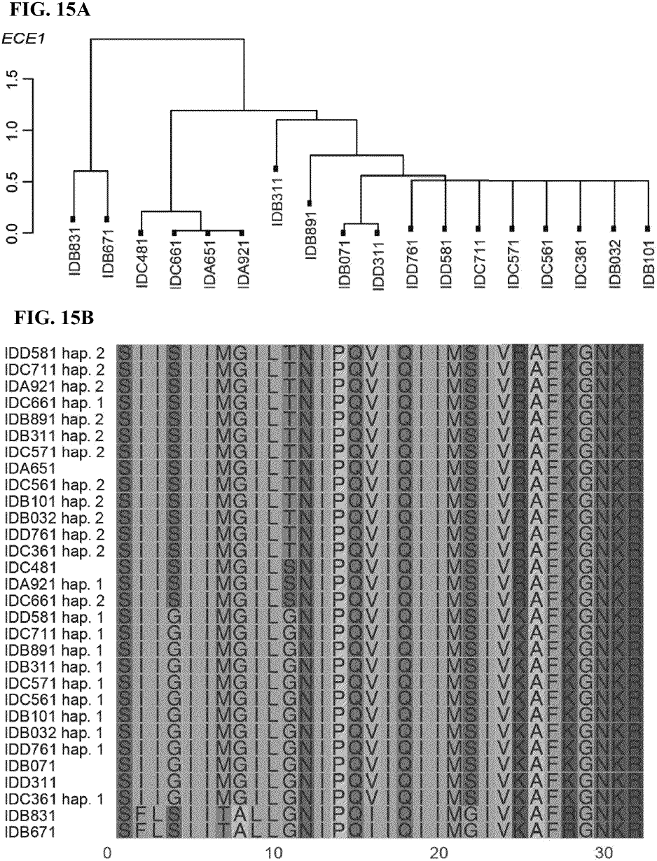

Recent studies revealed strong upregulation of genes encoding for hypha-associated virulence factors upon C. albicans intestinal colonization, including extent of cell elongation 1 gene (ECE1), secreted aspartic proteases genes SAP6, hyphally regulated gene 1 (HYR1) and agglutinin-like protein 3 precursor gene (ALS3), with ECE1 gene among the top hits³⁹. Notably, deletion in EFG1 gene almost fully inhibits ECE1 expression in an HD strain in vitro (FIG. 11D). Consistently, the analysis of a recently published dataset⁴⁵ revealed that ECE1 is among the top genes regulated by EFG1 during C. albicans colonization of the large intestine (FIG. 11E). Thus, we focused our attention on the ECE1-encoded peptide toxin, candidalysin, as a secreted factor that might drive the induction of immunity and cell damage^{32,35,36}. We first utilized a C. albicans ece1Δ/Δ mutant, which forms normal hyphae³⁵, and demonstrated a significantly lower level of macrophage and epithelial cell damage in vitro (FIGS. 14A-14B). We next sought to determine whether candidalysin plays a role in triggering proinflammatory immunity in the gut. Upon intestinal colonization, the ece1Δ/Δ strain induced significantly reduced colonic neutrophil infiltration and Th17 responses in the presence of gut bacteria in ASF-colonized mice in comparison with the C. albicans parental strain (FIGS. 14C-14D), despite comparable levels of gut colonization by both strains (FIG. 14E). Similarly, in the murine model of DSS-induced inflammation under prednisolone treatment, SPF mice intestinally colonized with C. albicans ece1Δ/Δ also exhibited significantly reduced intestinal inflammation, characterized by decreased infiltration of neutrophils and proinflammatory Th17 cells in the colon (FIGS. 14G-14J), despite similar levels of gut fungal colonization by both strains (FIG. 14F). Therefore, the hypha secreted toxin candidalysin is a critical factor driving intestinal inflammation by C. albicans.

To investigate the importance of candidalysin in clinical isolates responsible for inflammatory immunity in the gut, we generated ECE1 homozygous knockouts of HD and LD human gut C. albicans strains using the above CRISPR-based approach (FIG. 3A and FIGS. 3H-3I). Notably, HD/C.a ece1Δ/Δ strains (HD/C.a IDB131 ece1Δ/Δ and HD/C.a IDB101 ece1Δ/Δ) led to consistent reduction of cell damage in vitro (FIG. 3H). In addition, gut colonization by HD/C.a IDB131 ece1Δ/Δ resulted in a reduced Th17 recruitment and proinflammatory immunity in the colon of mice, similar to the phenotypes induced by the LD strain (LD/C.a IDC561) and the respective ece1Δ/Δ strain (LD/C.a IDC561 ece1Δ/Δ) (FIGS. 3J-3L). In contrast, ECE1 homozygous deletion in the LD strain only marginally reduced proinflammatory immunity as compared to the WT LD strain, demonstrating that candidalysin plays a key role in the initiation of inflammatory immunity in the HD strains (FIGS. 3J-3L). These properties were specific to ECE1 deletion and were not due to differences in fungal survival in vivo since similar fungal burdens (FIG. 3M) and mixed yeast/hyphae morphotypes were observed in the intestine among all groups (FIG. 13). These findings indicate that C. albicans-mediated intestinal immunity is strain-dependent, and that candidalysin plays a key role in this process in HD strains through Efg1-mediated regulation of an ECE1-dependent program.

Since phenotypic characterization allowed us to categorize authentic C. albicans gut isolates into two broad groups (LD/Ca and HD/Ca), we hypothesized that isolates with high immunoreactivity would be closely related as previously reported⁴⁹. Recent advances in C. albicans genomics have divided C. albicans into 17 emerging clades based on phylogeny^{28,50,51}. Thus, we sequenced 18 human gut C. albicans isolates, each obtained from a colonic mucosa-enriched sample of distinct individuals, and compared their whole genomes with the previously sequenced collection⁵¹ of C. albicans strains that were assigned to specific C. albicans clades (Supplementary Table D).

Supplemental TABLE D

Summary of whole genome sequenced C. albicans strains

| | | | | |
|---|---|---|---|---|
| C. albicans Strain | Site | Patient Origin | SNP.cluster | SRA.run.number |
| IDA651 | colon of UC patient | United States | 2 | PRJNA702809 |
| IDA652 | colon of UC patient | United States | 2 | PRJNA702809 |
| IDA653 | colon of UC patient | United States | 2 | PRJNA702809 |
| IDA921 | colon of UC patient | United States | 2 | PRJNA702809 |
| IDA922 | colon of UC patient | United States | 2 | PRJNA702809 |
| IDA923 | colon of UC patient | United States | 2 | PRJNA702809 |
| IDB311 | colon of UC patient | United States | NC | PRJNA702809 |
| IDB312 | colon of UC patient | United States | NC | PRJNA702809 |
| IDB313 | colon of UC patient | United States | NC | PRJNA702809 |
| IDB831 | colon of UC patient | United States | NC | PRJNA702809 |
| IDB832 | colon of UC patient | United States | NC | PRJNA702809 |
| IDB833 | colon of UC patient | United States | NC | PRJNA702809 |
| IDB032 | colon of Non-IBD individual | United States | 1 | PRJNA702809 |
| IDB071 | colon of UC patient | United States | 1 | PRJNA702809 |
| IDB072 | colon of UC patient | United States | 1 | PRJNA702809 |
| IDB073 | colon of UC patient | United States | 1 | PRJNA702809 |
| IDB101 | colon of UC patient | United States | 1 | PRJNA702809 |
| IDB102 | colon of UC patient | United States | 1 | PRJNA702809 |
| IDB104 | colon of UC patient | United States | 1 | PRJNA702809 |
| IDB671 | colon of Non-IBD individual | United States | C | PRJNA702809 |
| IDB891 | colon of Non-IBD individual | United States | NC | PRJNA702809 |
| IDC361 | colon of Non-IBD individual | United States | 1 | PRJNA702809 |
| IDC481 | colon of UC patient | United States | NC | PRJNA702809 |
| IDC482 | colon of UC patient | United States | NC | PRJNA702809 |
| IDC483 | colon of UC patient | United States | NC | PRJNA702809 |
| IDC561 | colon of Non-IBD individual | United States | 1 | PRJNA702809 |
| IDC562 | colon of Non-IBD individual | United States | 1 | PRJNA702809 |
| IDC563 | colon of Non-IBD individual | United States | 1 | PRJNA702809 |
| IDC571 | colon of UC patient | United States | 1 | PRJNA702809 |
| IDC661 | colon of UC patient | United States | 2 | PRJNA702809 |
| IDC711 | colon of UC patient | United States | 1 | PRJNA702809 |
| IDD311 | colon of Non-IBD individual | United States | 1 | PRJNA702809 |
| IDD581 | colon of Non-IBD individual | United States | 1 | PRJNA702809 |
| IDD582 | colon of Non-IBD individual | United States | 1 | PRJNA702809 |
| IDD761 | colon of Non-IBD individual | United States | 1 | PRJNA702809 |
| CEC3560 | Superficial | Niger | 1 | SRR6669916 |
| CEC4480 | Food spoilage | Unknown | 1 | SRR6669934 |
| CEC3623 | Vagina | Morocco | 1 | SRR6669884 |
| CEC4500 | Food spoilage | Unknown | 1 | SRR6669888 |
| CEC3544 | Mouth | Belgium | 1 | SRR6669962 |
| CEC3658 | Superficial | France | 1 | SRR6669900 |
| CEC4883 | Vagina | China | 1 | SRR6670022 |
| CEC4484 | Food spoilage | Unknown | 1 | SRR6669909 |
| CEC4107 | Superficial | Senegal | 1 | SRR6669933 |
| CEC3621 | Vagina | Brazil | 1 | SRR6669878 |
| CEC3612 | Vagina | Morocco | 2 | SRR6669942 |
| CEC3558 | Mouth | Belgium | 2 | SRR6669992 |
| CEC3669 | Superficial | France | 2 | SRR6670019 |
| CEC3531 | Mouth | France | 2 | SRR6669849 |
| CEC3615 | Vagina | Morocco | 2 | SRR6669941 |
| CEC4493 | Food spoilage | Unknown | 2 | SRR6669906 |
| CEC3614 | Vagina | Morocco | 2 | SRR6669940 |
| CEC4946 | Vagina | India | 2 | SRR6670002 |
| CEC4945 | Vagina | India | 2 | SRR6670003 |
| CEC4494 | Food spoilage | Unknown | 2 | SRR6669905 |
| CEC3626 | Mouth | Belgium | 3 | SRR6669883 |
| CEC3597 | Blood | France | 3 | SRR6669920 |
| CEC3579 | Mouth | France | 3 | SRR6669918 |
| CEC3637 | Mouth | Niger | 3 | SRR6669875 |
| CEC3681 | Blood | France | 3 | SRR6669865 |
| CEC709 | Mouth | France | 3 | SRR6669979 |
| CEC3557 | Digestive tract | Belgium | 3 | SRR6669991 |
| CEC3596 | Blood | France | 3 | SRR6669917 |
| CEC3638 | Mouth | Belgium | 3 | SRR6669899 |
| CEC1289 | Blood | France | 3 | SRR6670011 |
| CEC3675 | Blood | France | 4 | SRR6670014 |
| CEC2022 | Disseminated | France | 4 | SRR6669857 |
| CEC3610 | Vagina | Morocco | 4 | SRR6669936 |
| CEC3540 | Blood | France | 4 | SRR6669964 |
| CEC3530 | Mouth | France | 4 | SRR6669852 |
| CEC3674 | Urine | France | 4 | SRR6670015 |
| CEC1492 | Unknown | Unknown | 4 | SRR6670007 |
| CEC3535 | Vagina | Morocco | 4 | SRR6669967 |
| CEC3536 | Vagina | Morocco | 4 | SRR6669966 |
| CEC3532 | Mouth | France | 4 | SRR6669850 |
| CEC3622 | Vagina | Brazil | 8 | SRR6669877 |
| CEC3634 | Vagina | Brazil | 8 | SRR6669881 |
| CEC2023 | Feces | Guiana | 8 | SRR6669858 |
| CEC5026 | Vagina | UK | 9 | SRR6669994 |
| CEC2018 | Urine | France | 9 | SRR6670004 |
| CEC3533 | Mouth | France | 9 | SRR6669969 |
| CEC3668 | Urine | France | 9 | SRR6669904 |
| CEC3616 | Vagina | Morocco | 10 | SRR6669944 |
| CEC3706 | Urine | France | 10 | SRR6669959 |
| CEC3711 | Urine | France | 10 | SRR6669949 |
| CEC3547 | Mouth | Belgium | 11 | SRR6669972 |
| CEC3618 | Mouth | Belgium | 11 | SRR6669880 |
| CEC3601 | Mouth | Belgium | 11 | SRR6669922 |
| CEC4525 | Mouth | France | 11 | SRR6669889 |
| CEC4492 | Food spoilage | France | 11 | SRR6669911 |
| CEC4495 | Food spoilage | France | 11 | SRR6669891 |
| CEC3494 | Feces | Belgium | 11 | SRR6669854 |
| CEC3561 | Feces | Belgium | 11 | SRR6669915 |
| CEC1426 | Urine | France | 11 | SRR6670009 |
| CEC3704 | Urine | France | 11 | SRR6669862 |
| CEC3537 | Vagina | Morocco | 12 | SRR6669965 |
| CEC2019 | Mouth | Niger | 12 | SRR6670005 |
| CEC3680 | Blood | France | 12 | SRR6669983 |
| CEC3685 | Blood | France | 12 | SRR6669864 |
| CEC3686 | Blood | France | 12 | SRR6669861 |
| CEC716 | Mouth | France | 12 | SRR6669982 |
| CEC4878 | Vagina | China | 13 | SRR6669860 |
| CEC4882 | Vagina | China | 13 | SRR6670025 |
| CEC4103 | Vagina | Senegal | 13 | SRR6669961 |
| CEC4857 | Vagina | Angola | 13 | SRR6669868 |
| CEC4889 | Vagina | China | 13 | SRR6670027 |
| CEC5025 | Vagina | UK | 13 | SRR6669995 |
| CEC5023 | Vagina | UK | 13 | SRR6669997 |
| CEC5030 | Vagina | UK | 13 | SRR6669976 |
| CEC4943 | Vagina | India | 13 | SRR6670016 |
| CEC4860 | Vagina | China | 13 | SRR6669869 |
| CEC3664 | Urine | France | 16 | SRR6669898 |
| CEC3550 | Starling feces | France | 16 | SRR6669985 |
| CEC3600 | Starling feces | France | 16 | SRR6669919 |
| CEC2876 | Blood | South Korea | 18 | SRR6669853 |
| CEC2872 | Blood | South Korea | 18 | SRR6669856 |
| CEC3786 | Blood | South Korea | 18 | SRR6669957 |
| CEC2871 | Blood | South Korea | 18 | SRR6669855 |
| CEC3548 | GI tract | Belgium | A | SRR6669971 |
| CEC4038 | Wound | UK | A | SRR6669954 |
| CEC3715 | Urine | France | A | SRR6669955 |
| CEC4254 | Blood | France | B | SRR6669931 |
| CEC3708 | Urine | France | B | SRR6669946 |
| CEC3661 | Urine | France | C | SRR6669895 |
| CEC4039 | Sputum | UK | C | SRR6669953 |
| CEC4498 | Food spoilage | France | D | SRR6669894 |
| CEC3707 | Urine | France | D | SRR6669925 |
| CEC4486 | Food spoilage | France | E | SRR6669907 |
| CEC3712 | Urine | France | E | SRR6669956 |

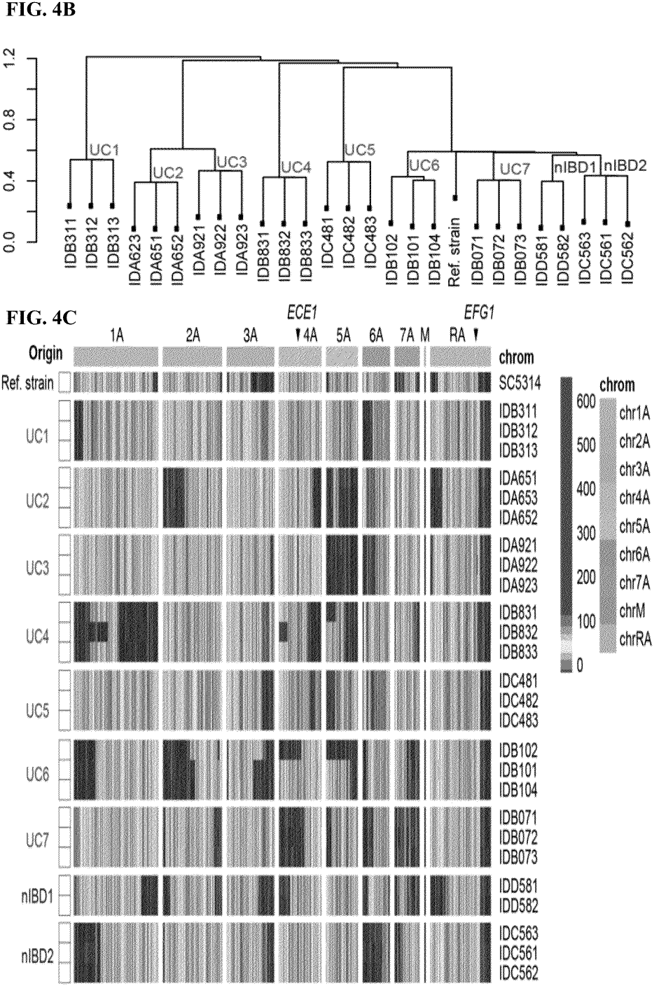

This analysis revealed a high diversity among gut C. albicans isolates with multiple clades represented irrespective of disease status (non-IBD or UC) (FIG. 4A). Surprisingly, gut C. albicans isolates from different individuals represented different strains, despite the close geographical location of our patient population (FIG. 4A), suggesting a wide genetic variety of gut C. albicans across individuals. These genetic basis adaptations were also observed in Candida spp. isolates from the lungs of patients with cystic fibrosis^{52,53.}

In the intestine, C. albicans undergoes clonal expansion and microevolution (generation-to-generation small-scale genetic changes in a population): two key genetic events occurring in the murine gut as reported by experimental studies using a model C. albicans strain³¹. Phylogenetic and heterozygous SNP density analysis revealed that several strains isolated from the gut of two individual patients (IDB10, IDB83) are derived from an ancestral strain of intestinal C. albicans, suggesting the possibility that microevolutionary events are also occurring in the human gut. Interestingly, we also found that clonal expansion is a common genetic event that occurred across the patient population (IDB31, IDA62, IDA92, IDC48, IDB07, IDD58) (FIGS. 4B-4C). While clonal expansion is a typical occurrence in a variety of invasive fungal diseases, these findings imply that parallel mechanisms of fungal expansion could be occurring during both classical fungal infections and inflammatory intestinal disease.

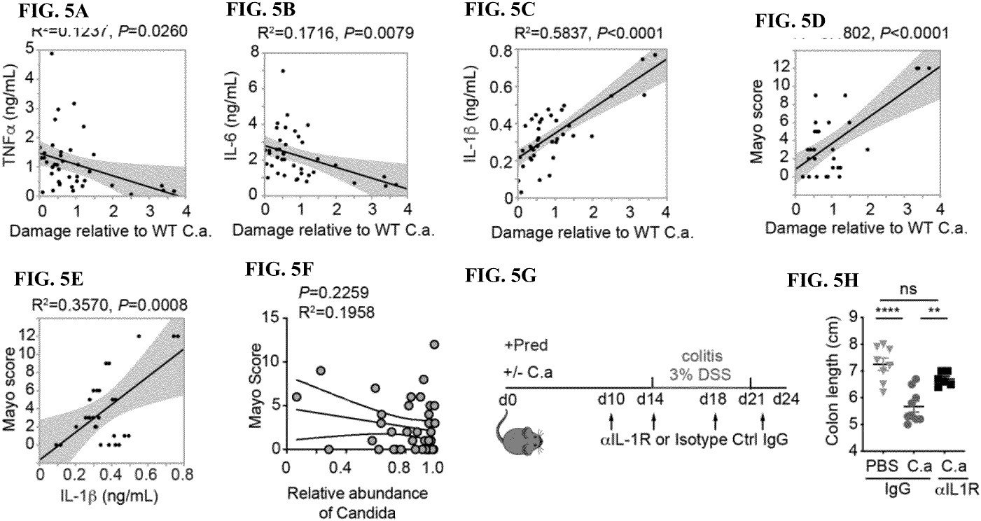

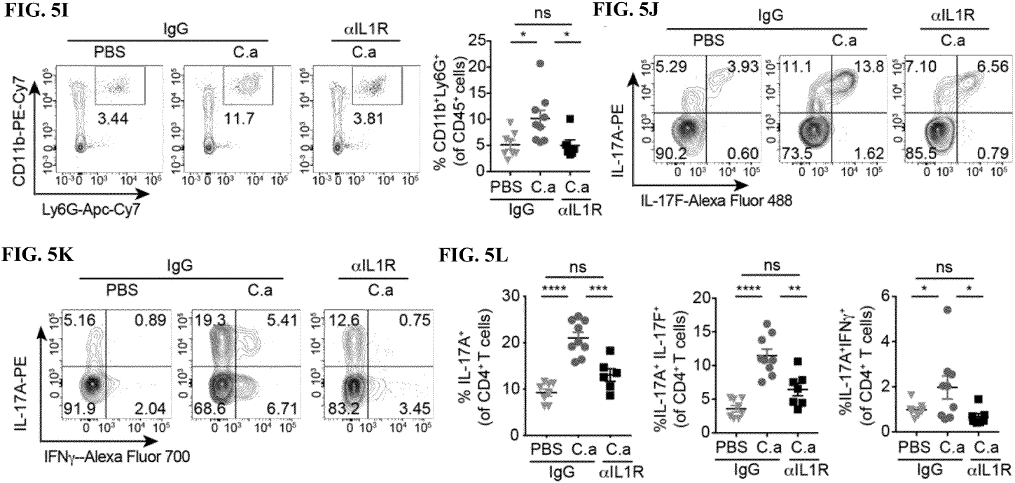

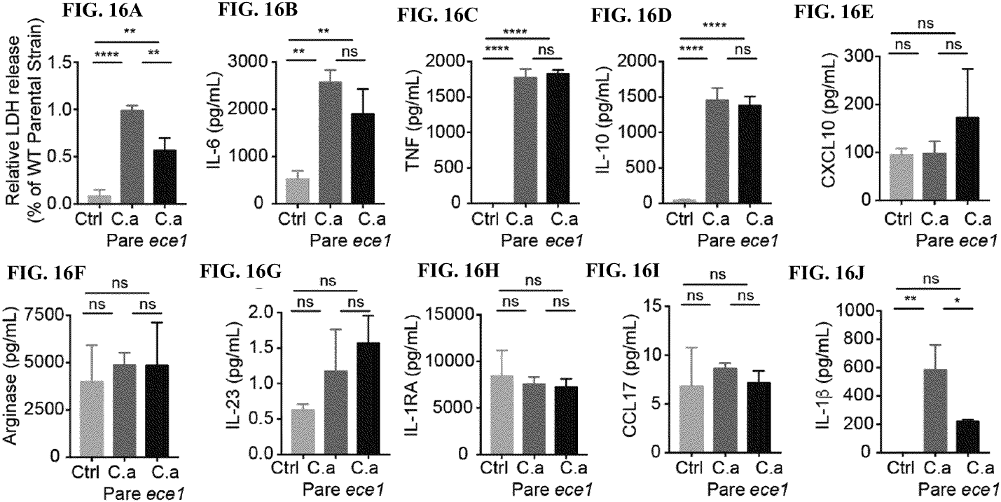

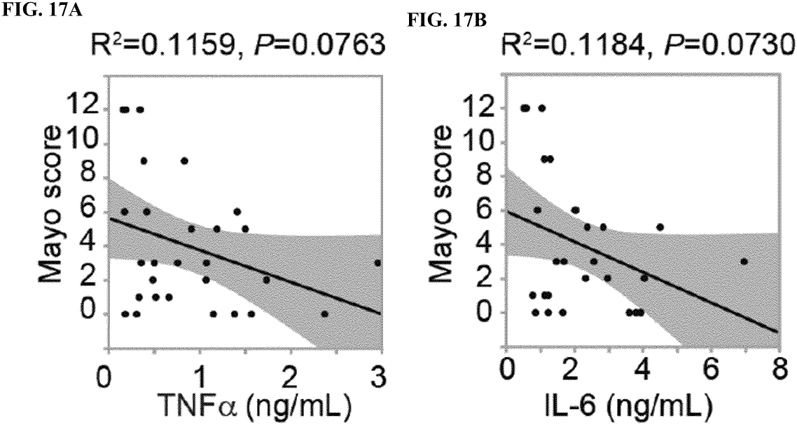

Given that C. albicans-produced candidalysin is a key factor in inflicting damage to human monocyte derived macrophages (hMDM) (FIG. 16A), we next sought to define whether pro-inflammatory mediators induced by different strains upon stimulation of hMDM are dependent on candidalysin. Among a panel of macrophage-released mediators, TNFα, IL-6, IL-1β and IL-10 were strongly induced by C. albicans infection. However, only IL-1β production was candidalysin dependent (FIGS. 16B-16J), thereby confirming its role as a driver of NLRP3-dependent IL-1β production by macrophages³²,⁵⁴. Furthermore IL-1β but not TNFα and IL-6 strongly correlated with the capacity of specific gut C. albicans strains to inflict immune cell damage (FIGS. 5A-5C).

We next assessed whether proinflammatory properties of specific strains correlated with disease severity in the patients of origin. This analysis revealed a strong correlation between the capacity of C. albicans strains to induce macrophage cell damage, IL-1β production and disease severity in individual UC patients (FIGS. 5D-5E). The observed phenomenon was specific to IL-1β since the magnitude of TNFα and IL-6 production upon stimulation with specific isolates did not correlate with UC severity (FIG. 5E, and FIGS. 17A-17B). In contrast, ITS-based compositional analysis failed to link the relative abundance of Candida with disease severity in individual patients (FIG. 5F), indicating that the functional characteristics of the strain, rather than fungal composition, determines Candida-related proinflammatory characteristics in individual patients. Thus, the capability of specific C. albicans strains to induce cell damage and IL-1β in phagocytes, but not the relative abundance of Candida, reflected the disease severity in the patient of origin.

IL-1β is an important driver of inflammation and is consistently increased in inflamed rectal biopsies from patients with UC², suggesting that IL-1β might be involved in the pathogenesis of UC^{2,55,56}. Nevertheless, the consequence of C. albicans-induced IL-1β on intestinal proinflammatory immunity in UC remains unknown. Notably, IL-1R blockade using an anti-IL-1R antibody dramatically reduced neutrophil recruitment, inflammatory Th17 cell accumulation and colonic inflammation in HD/C.a-colonized mice (FIGS. 5H-5L). This data indicates that IL-1 signaling plays a critical role in promoting intestinal C. albicans-induced proinflammatory immune cell accumulation in the colon. Remarkably, the presence of gut C. albicans strains with high ability to induce IL-1β production in phagocytes positively correlated with increased disease severity in their host, suggesting that C. albicans strains from these patients with severe UC have acquired or retained the ability to secrete candidalysin and cause cell damage and inflammation.

Taken together, these results demonstrate that candidalysin is a necessary damaging factor that fuels tissue proinflammatory immunity and determines the pathogenicity of C. albicans strains in the gut. These results also show that such strains with high damaging capacity propels intestinal inflammation in vivo through IL-1β-dependent mechanisms, and patients carrying high-damaging strains are thus suitable for IL-1-blocking therapy and/or antifungal co-therapy.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

### REFERENCES

1. Human Microbiome Project, C. Structure, function and diversity of the healthy human microbiome. Nature 486, 207-14 (2012).

2. Lloyd-Price, J. et al. Multi-omics of the gut microbial ecosystem in inflammatory bowel diseases. Nature 569, 655-662 (2019).

3. Chehoud, C. et al. Fungal Signature in the Gut Microbiota of Pediatric Patients With Inflammatory Bowel Disease. Inflammatory bowel diseases 21, 1948-56 (2015).

4. Hoarau, G. et al. Bacteriome and Mycobiome Interactions Underscore Microbial Dysbiosis in Familial Crohn’s Disease. mBio 7, e01250-16 (2016).

5. Liguori, G. et al. Fungal Dysbiosis in Mucosa-associated Microbiota of Crohn’s Disease Patients. Journal of Crohn’s & colitis 10, 296-305 (2016).

6. Sokol, H. et al. Fungal microbiota dysbiosis in IBD. Gut 66, 1039-1048 (2017).

7. Ott, S. J. et al. Fungi and inflammatory bowel diseases: Alterations of composition and diversity. Scand J Gastroenterol 43, 831-41 (2008).

8. Limon, J. J. et al. Malassezia Is Associated with Crohn’s Disease and Exacerbates Colitis in Mouse Models. Cell host & microbe (2019) doi:10.1016/j.chom.2019.01.007.

9. Jain, U. et al. Debaryomyces is enriched in Crohn’s disease intestinal tissue and impairs healing in mice. Science 371, 1154-1159 (2021).

10. Kaplan, G. G. The global burden of IBD: from 2015 to 2025. Nature reviews. Gastroenterology & hepatology 12, 720-7 (2015).

11. Israeli, E. et al. Anti-Saccharomyces cerevisiae and antineutrophil cytoplasmic antibodies as predictors of inflammatory bowel disease. Gut 54, 1232-6 (2005).

12. Lewis, J. D. et al. Inflammation, Antibiotics, and Diet as Environmental Stressors of the Gut Microbiome in Pediatric Crohn’s Disease. Cell host & microbe 18, 489-500 (2015).

13. Schaffer, T. et al. Anti-Saccharomyces cerevisiae mannan antibodies (ASCA) of Crohn’s patients crossreact with mannan from other yeast strains, and murine ASCA IgM can be experimentally induced with Candida albicans. Inflammatory bowel diseases 13, 1339-46 (2007).

14. Standaert-Vitse, A. et al. Candida albicans colonization and ASCA in familial Crohn’s disease. Am J Gastroenterol 104, 1745-53 (2009).

15. Leonardi, I. et al. CX3CR1(+) mononuclear phagocytes control immunity to intestinal fungi. Science 359, 232-236 (2018).

16. Cohen, R., Roth, F. J., Delgado, E., Ahearn, D. G. & Kalser, M. H. Fungal flora of the normal human small and large intestine. The New England journal of medicine 280, 638-41 (1969).

17. Iliev, I. D. Dectin-1 Exerts Dual Control in the Gut. Cell host & microbe 18, 139-141 (2015).

18. Iliev, I. D. et al. Interactions between commensal fungi and the C-type lectin receptor Dectin-1 influence colitis. Science 336, 1314-7 (2012).

19. Sovran, B. et al. Enterobacteriaceae are essential for the modulation of colitis severity by fungi. Microbiome 6, 152 (2018).

20. Danese, S. & Fiocchi, C. Ulcerative Colitis. N Engl J Med 365, 1713-1725 (2011).

21. Fan, D. et al. Activation of HIF-1alpha and LL-37 by commensal bacteria inhibits Candida albicans colonization. Nature medicine 21, 808-14 (2015).

22. Jawhara, S. et al. Colonization of mice by Candida albicans is promoted by chemically induced colitis and augments inflammatory responses through galectin-3. J Infect Dis 197, 972-80 (2008).

23. Caballero, S. et al. Cooperating Commensals Restore Colonization Resistance to Vancomycin-Resistant Enterococcus faecium. Cell host & microbe 21, 592-602 e4 (2017).

24. Poyet, M. et al. A library of human gut bacterial isolates paired with longitudinal multiomics data enables mechanistic microbiome research. Nature medicine 25, 1442-1452 (2019).

25. Lagier, J. C. et al. Culturing the human microbiota and culturomics. Nature reviews. Microbiology 540-550 (2018) doi: 10.1038/s41579-018-0041-0.

26. Kim, S. G. et al. Microbiota-derived lantibiotic restores resistance against vancomycin-resistant Enterococcus. Nature 572, 665-+ (2019).

27. Marakalala, M. J. et al. Differential adaptation of Candida albicans in vivo modulates immune recognition by dectin-1. PLoS pathogens 9, e1003315 (2013).

28. Hirakawa, M. P. et al. Genetic and phenotypic intra-species variation in Candida albicans. Genome Res 25, 413-25 (2015).

29. Schonherr, F. A. et al. The intraspecies diversity of C-albicans triggers qualitatively and temporally distinct host responses that determine the balance between commensalism and pathogenicity. Mucosal Immunol 10, 1335-1350 (2017).

30. Forche, A. et al. Selection of Candida albicans trisomy during oropharyngeal infection results in a commensal-like phenotype. PLoS Genet 15, e1008137 (2019).

31. Tso, G. H. W. et al. Experimental evolution of a fungal pathogen into a gut symbiont. Science 362, 589-595 (2018).

32. Kasper, L. et al. The fungal peptide toxin candidalysin activates the NLRP3 inflammasome and causes cytolysis in mononuclear phagocytes. Nature communications 9, 4260 (2018).

33. Wellington, M., Koselny, K., Sutterwala, F. S. & Krysan, D. J. Candida albicans triggers NLRP3-mediated pyroptosis in macrophages. Eukaryot Cell 13, 329-40 (2014).

34. Uwamahoro, N. et al. The Pathogen Candida albicans Hijacks Pyroptosis for Escape from Macrophages. mBio 5, (2014).

35. Moyes, D. L. et al. candidalysin is a fungal peptide toxin critical for mucosal infection. Nature 532, 64-8 (2016).

36. Verma, A. H. et al. Oral epithelial cells orchestrate innate type 17 responses to Candida albicans through the virulence factor candidalysin. Science immunology 2, (2017).

37. Naglik, J. R., Gaffen, S. L. & Hube, B. candidalysin: discovery and function in Candida albicans infections. Curr Opin Microbiol 52, 100-109 (2019).

38. Pierce, J. V. & Kumamoto, C. A. Variation in Candidaalbicans EFG1 expression enables host-dependent changes in colonizing fungal populations. mBio 3, e00117-12 (2012).

39. Witchley, J. N. et al. Candida albicans Morphogenesis Programs Control the Balance between Gut Commensalism and Invasive Infection. Cell host & microbe 25, 432-443 e6 (2019).

40. Li, X. et al. Response to Fungal Dysbiosis by Gut-Resident CX3CR1(+) Mononuclear Phagocytes Aggravates Allergic Airway Disease. Cell host & microbe 24, 847-856 e4 (2018).

41. Doron, I., Leonardi, I. & Iliev, I. D. Profound mycobiome differences between segregated mouse colonies do not influence Th17 responses to a newly introduced gut fungal commensal. Fungal genetics and biology : FG & B (2019) doi: 10.1016/j.fgb.2019.03.001.

42. Doron, I. et al. Human gut mycobiota tune immunity via CARD9-dependent induction of anti-fungal IgG antibodies. Cell 184, 1017-1031.e14 (2021).

43. Rohde, C. M. et al. Metabonomic evaluation of Schaedler altered microflora rats. Chem Res Toxicol 20, 1388-92 (2007).

44. Schaedler, R. W., Dubos, R. & Costello, R. The Development of the Bacterial Flora in the Gastrointestinal Tract of Mice. The Journal of experimental medicine 122, 59-66 (1965).

45. Witchley, J. N., Basso, P., Brimacombe, C. A., Abon, N. V. & Noble, S. M. Recording of DNA-binding events reveals the importance of a repurposed Candida albicans regulatory network for gut commensalism. Cell Host & Microbe 29, 1002-1013.e9 (2021).

46. Vyas, V. K. et al. New CRISPR Mutagenesis Strategies Reveal Variation in Repair Mechanisms among Fungi. mSphere 3, (2018).

47. Vyas, V. K., Barrasa, M. I. & Fink, G. R. A Candida albicans CRISPR system permits genetic engineering of essential genes and gene families. Sci Adv 1, e1500248 (2015).

48. Selmecki, A., Forche, A. & Berman, J. Genomic plasticity of the human fungal pathogen Candida albicans. Eukaryot Cell 9, 991-1008 (2010).

49. MacCallum, D. M. et al. Property differences among the four major Candida albicans strain clades. Eukaryot Cell 8, 373-87 (2009).

50. Butler, G. et al. Evolution of pathogenicity and sexual reproduction in eight Candida genomes. Nature 459, 657-62 (2009).

51. Ropars, J. et al. Gene flow contributes to diversification of the major fungal pathogen Candida albicans. Nature communications 9, 2253 (2018).

52. Kim, S. H. et al. Global Analysis of the Fungal Microbiome in Cystic Fibrosis Patients Reveals Loss of Function of the Transcriptional Repressor Nrg1 as a Mechanism of Pathogen Adaptation. PLoS pathogens 11, e1005308 (2015).

53. Demers, E. G. et al. Evolution of drug resistance in an antifungal-naive chronic Candida lusitaniae infection. Proc Natl Acad Sci USA 115, 12040-12045 (2018).

54. Drummond, R. A. et al. CARD9(+) microglia promote antifungal immunity via IL-1beta-and CXCL1-mediated neutrophil recruitment. Nature immunology 20, 559-570 (2019).

55. Shouval, D. S. et al. Interleukin 1beta Mediates Intestinal Inflammation in Mice and Patients With Interleukin 10 Receptor Deficiency. Gastroenterology 151, 1100-1104 (2016).

56. Friedrich, M. et al. IL-1-driven stromal-neutrophil interaction in deep ulcers defines a pathotype of therapy non-responsive inflammatory bowel disease. http://biorxiv.org/lookup/doi/10.1101/2021.02.05.429804 (2021) doi: 10.1101/2021.02.05.429804.

57. Sendid, B. et al. Anti-Saccharomyces cerevisiae mannan antibodies in familial Crohn’s disease. Am J Gastroenterol 93, 1306-10 (1998).

58. Soderholm, J. D. et al. Different intestinal permeability patterns in relatives and spouses of patients with Crohn’s disease: an inherited defect in mucosal defence? Gut 44, 96-100 (1999).

59. Mogavero, S. et al. candidalysin delivery to the invasion pocket is critical for host epithelial damage induced by Candida albicans. Cellular Microbiology 23, (2021).

60. Longman, R. S. et al. CX(3)CR1(+) mononuclear phagocytes support colitis-associated innate lymphoid cell production of IL-22. The Journal of experimental medicine 211, 1571-83 (2014).

61. Hepworth, M. R. et al. Immune tolerance. Group 3 innate lymphoid cells mediate intestinal selection of commensal bacteria-specific CD4(+) T cells. Science 348, 1031-5 (2015).

62. Tang, J., Iliev, I. D., Brown, J., Underhill, D. M. & Funari, V. A. Mycobiome: Approaches to analysis of intestinal fungi. J Immunol Methods 421, 112-121 (2015).

63. Altschul, S. F., Gish, W., Miller, W., Myers, E. W. & Lipman, D. J. Basic local alignment search tool. J Mol Biol 215, 403-10 (1990).

64. Li, H. & Durbin, R. Fast and accurate short read alignment with Burrows-Wheeler transform. Bioinformatics 25, 1754-60 (2009).

65. McKenna, A. et al. The Genome Analysis Toolkit: a MapReduce framework for analyzing next-generation DNA sequencing data. Genome Res 20, 1297-303 (2010).

66. Zheng, X. et al. A high-performance computing toolset for relatedness and principal component analysis of SNP data. Bioinformatics 28, 3326-8 (2012).

67. Gu, Z., Gu, L., Eils, R., Schlesner, M. & Brors, B. circlize Implements and enhances circular visualization in R. Bioinformatics 30, 2811-2 (2014).

68. Danecek, P. et al. Twelve years of SAMtools and BCFtools. Gigascience 10, (2021).

69. Quinlan, A. R. & Hall, I. M. BEDTools: a flexible suite of utilities for comparing genomic features. Bioinformatics 26, 841-842 (2010).

70. Bankevich, A. et al. SPAdes: a new genome assembly algorithm and its applications to single-cell sequencing. J Comput Biol 19, 455-477 (2012).

71. Wick, R. R., Schultz, M. B., Zobel, J. & Holt, K. E. Bandage: interactive visualization of de novo genome assemblies. Bioinformatics 31, 3350-3352 (2015).

1. A method for treating a patient suffering from a fungal-associated intestinal inflammatory disorder comprising administering to the patient an effective amount of an IL-1 pathway inhibitor, wherein gut tissue of the patient comprises a population of candidalysin-secreting C. albicans.

2. A method for selecting a patient suffering from a fungal-associated intestinal inflammatory disorder for treatment with an IL-1 pathway inhibitor comprising
(a) detecting the presence of candidalysin-secreting C. albicans in a biological sample obtained from the patient; and
(b) administering to the patient an effective amount of an IL-1 pathway inhibitor.

3. The method of claim 2, wherein the biological sample is a colonic mucosa-enriched lavage sample, a fecal sample, a rectal swab, or an intestinal sample.

4. The method of claim 1, wherein the fungal-associated intestinal inflammatory disorder is inflammatory bowel disease (IBD), Crohn’s disease (CD), or ulcerative colitis (UC).

5. The method of claim 1, wherein the IL-1 pathway inhibitor is an inflammasome-blocking drug, an anti-IL-1R1 antibody or antigen binding fragment, Anakinra, Rilonacept, Canakinumab, Gevokizumab, LY2189102, MABp1, MEDI-8968, CYT013, sIL-1RI, sIL-1RII, EBI-005, CMPX-1023, MCC950, Inzomelid, Somalix, NT-0167, IFM-2427 (DFV890), Dapansutrile (OLT1177), glyburide, 16673-34-0, JC124, FC11A-2, parthenolide, Bay 11-7082, BHB, MNS, CY-09, tranilast, oridonin, VX-740, or VX-765.

6. The method of claim 1, wherein the candidalysin-secreting C. albicans expresses elevated enhanced filamentous growth protein 1 (EFG1) expression compared to a reference non-filamentous C. albicans strain or a predetermined threshold.

7. The method of claim 1, wherein the candidalysin-secreting C. albicans expresses increased hyphae production relative to a reference non-filamentous C. albicans strain.

8. The method of claim 1, wherein the candidalysin-secreting C. albicans expresses elevated expression levels of at least one protease selected from among SAP6, SAP5, or SAP2 compared to a reference non-filamentous C. albicans strain or a predetermined threshold.

9. The method of claim 1, wherein the candidalysin-secreting C. albicans expresses elevated expression levels of ALS3 or ALS1 compared to a reference non-filamentous C. albicans strain or a predetermined threshold.

10. The method of claim 6, wherein the reference non-filamentous C. albicans strain is an efg1Δ/Δ C. albicans mutant strain.

11. The method of claim 1, wherein the candidalysin-secreting C. albicans induces an in vivo proinflammatory response in host cells.

12. The method of claim 11, wherein the in vivo proinflammatory responses comprises neutrophil infiltration and/or Th17 responses in the colon of the patient.

13. A kit comprising
(a) an expression vector comprising a nucleic acid sequence encoding a Candida-compatible Cas9 nuclease and a nucleic acid sequence encoding a synthetic guide RNA (sgRNA) that is configured to cleave a region in a target gene of at least one C. albicans strain that resides in human gut tissue, wherein the target gene is associated with high immune cell-damaging capacity and wherein the at least one C. albicans strain induces proinflammatory immunity in a human subject; and
(b) a heterologous repair template nucleic acid sequence comprising (i) a 5′ region that is homologous to a C. albicans nucleic acid sequence that is upstream or downstream from the region in the target gene that is cleaved by the sgRNA and (ii) a 3′ region comprising an open reading frame (ORF) deletion of the target gene.

14. The kit of claim 13, wherein the 5′ region of the heterologous repair template nucleic acid sequence is about 60 base pairs in length.

15. The kit of claim 13, wherein the 3′ region of the heterologous repair template nucleic acid sequence is about 20 base pairs in length.

16. The kit of claim 13, wherein the human subject is suffering from inflammatory bowel disease.


SEQUENCE LISTING (36 sequences)

SEQ ID NO: 1 - DNA, 26 bp, synthetic construct
  source 1..26: mol_type = other DNA
atttgtcacaacagccaccactaccg

SEQ ID NO: 2 - DNA, 26 bp, synthetic construct
  source 1..26: mol_type = other DNA
aaaacggtagtggtggctgttgtgac

SEQ ID NO: 3 - DNA, 26 bp, synthetic construct
  source 1..26: mol_type = other DNA
atttgaatttctggcaatctgacgag

SEQ ID NO: 4 - DNA, 26 bp, synthetic construct
  source 1..26: mol_type = other DNA
aaaactcgtcagattgccagaaattc

SEQ ID NO: 5 - DNA, 60 bp, synthetic construct
  source 1..60: mol_type = other DNA
aacgaattaagatttgttctatttgactaccaagaatataacccatattataaatatcat

SEQ ID NO: 6 - DNA, 60 bp, synthetic construct
  source 1..60: mol_type = other DNA
tttggaatttatggcagaaagcagaaggtgatgtacacaaatgatatttataatatgggt

SEQ ID NO: 7 - DNA, 60 bp, synthetic construct
  source 1..60: mol_type = other DNA
aacaaacaactttcctttattttactaccaactattttccattcgttaaaatgctcagca

SEQ ID NO: 8 - DNA, 60 bp, synthetic construct
  source 1..60: mol_type = other DNA
tggaataaaagattaagcttgtggaaaacaaatttttatctgctgagcattttaacgaat

SEQ ID NO: 9 - DNA, 22 bp, synthetic construct
  source 1..22: mol_type = other DNA
ggcatagctgaaacttcggccc

SEQ ID NO: 10 - DNA, 21 bp, synthetic construct
  source 1..21: mol_type = other DNA
caattcaaaacgaattgcacc

SEQ ID NO: 11 - DNA, 20 bp, synthetic construct
  source 1..20: mol_type = other DNA
atatcaatacagcgagccat

SEQ ID NO: 12 - DNA, 20 bp, synthetic construct
  source 1..20: mol_type = other DNA
aattcattaccaggcgtgtt

SEQ ID NO: 13 - DNA, 20 bp, synthetic construct
  source 1..20: mol_type = other DNA
cgttcatgtcaatggatttg

SEQ ID NO: 14 - DNA, 23 bp, synthetic construct
  source 1..23: mol_type = other DNA
cttcaaagactcccacaactcat

SEQ ID NO: 15 - DNA, 26 bp, synthetic construct
  source 1..26: mol_type = other DNA
agcttttccgaaatattcttcaatca

SEQ ID NO: 16 - DNA, 22 bp, synthetic construct
  source 1..22: mol_type = other DNA
cattgatccgattttcacaacg

SEQ ID NO: 17 - DNA, 22 bp, synthetic construct
  source 1..22: mol_type = other DNA
cctacaacagatatcagtatcc

SEQ ID NO: 18 - DNA, 23 bp, synthetic construct
  source 1..23: mol_type = other DNA
tcagaccagctgatttaggtttg

SEQ ID NO: 19 - DNA, 20 bp, synthetic construct
  source 1..20: mol_type = other DNA
gtgaacaatggatggaccag

SEQ ID NO: 20 - DNA, 22 bp, synthetic construct
  source 1..22: mol_type = other DNA
cttggtcatttagaggaagtaa

SEQ ID NO: 21 - DNA, 20 bp, synthetic construct
  source 1..20: mol_type = other DNA
gctgcgttcttcatcgatgc

SEQ ID NO: 22 - DNA, 33 bp, synthetic construct
  source 1..33: mol_type = other DNA
tcgtcggcagcgtcagatgtgtataagagacag

SEQ ID NO: 23 - DNA, 34 bp, synthetic construct
  source 1..34: mol_type = other DNA
gtctcgtgggctcggagatgtgtataagagacag

SEQ ID NO: 24 - DNA, 19 bp, synthetic construct
  source 1..19: mol_type = other DNA
  modified_base 1: mod_base = OTHER; note = 5' cyanine 3 modified nucleotide
ctctggcttcaccctattc

SEQ ID NO: 25 - protein, 1371 aa, Candida sp.
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEGA

SEQ ID NO: 26 - protein, 1371 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEGA

SEQ ID NO: 27 - DNA, 4103 bp, Candida sp.
  source 1..4103: mol_type = genomic DNA
atggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgac

SEQ ID NO: 28 - DNA, 4113 bp, synthetic construct
  source 1..4113: mol_type = other DNA
atggataaaaagtatagtattggtttagatattggtactaactctgtgggttgggcagttatcaccgacgaatataaagttccatcaaagaaatttaaggtgttaggtaacactgacagacactcaataaaaaagaatcttatcggtgctcttttgttcgactccggtgaaactgccgaggctacacgtttaaaaagaacagcaagaagaagatatacccgtagaaaaaatagaatatgttatttacaagaaatcttttctaatgaaatggctaaagttgatgattcctttttccatagattggaagagtcatttttggttgaagaagacaaaaagcatgagagacatccaatctttgggaatatagttgatgaagtggcttaccatgaaaaatatcctaccatttatcatttaagaaagaaattggtagattcaactgataaagctgaccttagattaatctatttagcacttgcccatatgattaaatttagaggtcattttttgattgaaggtgatttgaacccagataattctgacgtggataaattatttattcaattagtccaaacctacaaccaattatttgaggaaaatccaattaatgctagtggtgtcgatgccaaagctatattatcagccagattatcaaaatctagacgtttggaaaatttgattgcccaattgccaggagaaaaaaagaatggattatttggaaacttgatcgcattatcattgggtttgacaccaaattttaaatctaattttgatttagctgaagatgctaaattacaattatcaaaagacacctatgacgacgatttggacaatttacttgctcaaattggtgatcaatatgcagatttgttcttagctgctaaaaacttatctgatgctattttgttgtctgatattttgagagtgaacacagaaataaccaaagctccattatcagcatctatgatcaaacgttatgatgaacaccatcaggatttgactttattgaaagctttggtgagacaacaattgccagagaagtataaagaaatctttttcgatcaatctaaaaacgggtatgcaggttatattgatgggggtgcctcccaagaggaattttacaaatttataaaacctattttagaaaagatggatgggactgaggaacttttggtcaaattgaacagagaagatttgttacgtaaacagagaacttttgataatggtagtatacctcaccaaattcatttgggtgagttgcatgcaattttaagaagacaagaagatttttatccatttttaaaagataatagagaaaaaatcgagaaaattttaacctttagaattccatactatgttgggcctttggctagaggtaattcaagatttgcctggatgacacgtaaatcagaagaaactattaccccttggaattttgaagaggttgttgataaaggagcatcagcacagagttttattgaaagaatgaccaatttcgataaaaacttaccaaatgaaaaagttttaccaaaacattccttgttatacgaatattttactgtttacaatgaacttacaaaggttaaatatgttactgaaggtatgcgtaagccagcctttttatctggagaacagaaaaaggcaatagttgatttattgtttaaaacaaatagaaaagttactgttaaacaattaaaagaagattactttaagaaaattgaatgttttgattcagttgaaatcagtggtgttgaagacagatttaatgctagtttaggaacttaccatgatttacttaaaattatcaaagataaagatttcttggataacgaagaaaatgaagacattttagaagacattgttttaaccttaactttattcgaagatagagagatgattgaagaacgtttgaagacttatgcacatttgtttgacgataaagtgatgaaacagttgaaaagaagacgttatactggatggggtagattgtctcgtaaattgatcaatggaattagagataaacaaagtggtaaaactatcttggactttttgaaatctgacggatttgctaatagaaatttcatgcaattgatccacgacgatagtttgacatttaaagaagacatccaaaaggcccaagtgagtgggcaaggtgattcattacatgaacatattgcaaatttagccggatctcctgctattaagaaagggatattacaaactgttaaagttgtggatgaattagtgaaagtaatgggaagacataaacctgaaaacattgtcattgagatggcaagagaaaatcaaactacacaaaaaggacagaaaaatagtagagaacgtatgaaaagaatagaagagggtattaaagaattgggtagtcaaatattgaaagaacacccagtggaaaatacccagttgcaaaatgaaaaattatatctttactaccttcaaaatggacgtgatatgtatgttgatcaggaattagatataaatagactttcagattatgatgtagatcatatagttccacaatctttcttgaaagatgattccatagacaataaagtattaactagaagtgataaaaatagaggtaaaagtgataatgtcccaagtgaggaagtcgtcaaaaagatgaaaaattactggcgtcaacttttgaatgctaaattaattactcaaagaaaatttgataatttgactaaagcagaaagaggtgggctttctgaattagataaagccgggttcattaaaagacaattggtcgaaactagacaaattactaaacatgttgcccaaattttagattcccgtatgaacactaagtatgacgaaaatgataagttaatacgtgaggttaaagtcattactttaaaatcaaaacttgtctctgatttcagaaaggatttccaattctataaagttagagaaattaataattatcatcatgctcatgatgcatatttgaatgctgtagttggaactgctttaatcaagaaataccctaaattagaatctgaatttgtatatggtgattacaaagtctatgatgttagaaagatgattgctaaatcagaacaagaaattggtaaagctacagctaaatacttcttttactctaacattatgaatttctttaaaacagaaattactttggcaaacggtgaaattagaaaaagacctcttattgaaacaaatggtgagactggagagatagtttgggacaaagggcgtgatttcgctactgttagaaaagttttatcaatgccacaagttaacattgtaaagaaaacagaggttcaaactggtggtttctcaaaagaaagtattttgcctaaaagaaatagtgataaattgattgccagaaaaaaggattgggatccaaagaaatatggtggtttcgactcaccaaccgtagcctattctgttttggttgtggcaaaggttgaaaagggtaaaagtaaaaagcttaaatcagtaaaagaacttttgggtattacaataatggaaagaagttcctttgaaaagaaccctattgattttttggaagctaaaggttataaggaagtaaagaaggacttaataatcaaattgcctaaatattctttatttgaattagaaaatgggagaaaaagaatgttggcttctgctggagaattgcaaaagggtaatgaattagcattgccttccaaatatgttaacttcttgtatttagcttcacactatgaaaagttgaaagggtcaccagaagataacgagcaaaaacaattatttgttgaacaacacaaacactacttagatgagattatagaacaaattagtgaattcagtaaaagagtgatattagctgatgcaaatttagataaagttttgtcagcctataacaaacatagagataagccaattagagaacaagcagaaaacattattcacttatttacccttaccaatttaggagcacctgctgctttcaagtattttgatacaacaattgatcgtaaaagatatacctcaacaaaagaagtcttagacgccaccttaattcatcaatcaatcactggattgtatgagacaagaattgatttgtctcaattgggtggtgatgaaggggct

SEQ ID NO: 29 - DNA, 4113 bp, synthetic construct
  source 1..4113: mol_type = other DNA
atggataaaaagtatagtattggtttagctattggtactaactctgtgggttgggcagttatcaccgacgaatataaagttccatcaaagaaatttaaggtgttaggtaacactgacagacactcaataaaaaagaatcttatcggtgctcttttgttcgactccggtgaaactgccgaggctacacgtttaaaaagaacagcaagaagaagatatacccgtagaaaaaatagaatatgttatttacaagaaatcttttctaatgaaatggctaaagttgatgattcctttttccatagattggaagagtcatttttggttgaagaagacaaaaagcatgagagacatccaatctttgggaatatagttgatgaagtggcttaccatgaaaaatatcctaccatttatcatttaagaaagaaattggtagattcaactgataaagctgaccttagattaatctatttagcacttgcccatatgattaaatttagaggtcattttttgattgaaggtgatttgaacccagataattctgacgtggataaattatttattcaattagtccaaacctacaaccaattatttgaggaaaatccaattaatgctagtggtgtcgatgccaaagctatattatcagccagattatcaaaatctagacgtttggaaaatttgattgcccaattgccaggagaaaaaaagaatggattatttggaaacttgatcgcattatcattgggtttgacaccaaattttaaatctaattttgatttagctgaagatgctaaattacaattatcaaaagacacctatgacgacgatttggacaatttacttgctcaaattggtgatcaatatgcagatttgttcttagctgctaaaaacttatctgatgctattttgttgtctgatattttgagagtgaacacagaaataaccaaagctccattatcagcatctatgatcaaacgttatgatgaacaccatcaggatttgactttattgaaagctttggtgagacaacaattgccagagaagtataaagaaatctttttcgatcaatctaaaaacgggtatgcaggttatattgatgggggtgcctcccaagaggaattttacaaatttataaaacctattttagaaaagatggatgggactgaggaacttttggtcaaattgaacagagaagatttgttacgtaaacagagaacttttgataatggtagtatacctcaccaaattcatttgggtgagttgcatgcaattttaagaagacaagaagatttttatccatttttaaaagataatagagaaaaaatcgagaaaattttaacctttagaattccatactatgttgggcctttggctagaggtaattcaagatttgcctggatgacacgtaaatcagaagaaactattaccccttggaattttgaagaggttgttgataaaggagcatcagcacagagttttattgaaagaatgaccaatttcgataaaaacttaccaaatgaaaaagttttaccaaaacattccttgttatacgaatattttactgtttacaatgaacttacaaaggttaaatatgttactgaaggtatgcgtaagccagcctttttatctggagaacagaaaaaggcaatagttgatttattgtttaaaacaaatagaaaagttactgttaaacaattaaaagaagattactttaagaaaattgaatgttttgattcagttgaaatcagtggtgttgaagacagatttaatgctagtttaggaacttaccatgatttacttaaaattatcaaagataaagatttcttggataacgaagaaaatgaagacattttagaagacattgttttaaccttaactttattcgaagatagagagatgattgaagaacgtttgaagacttatgcacatttgtttgacgataaagtgatgaaacagttgaaaagaagacgttatactggatggggtagattgtctcgtaaattgatcaatggaattagagataaacaaagtggtaaaactatcttggactttttgaaatctgacggatttgctaatagaaatttcatgcaattgatccacgacgatagtttgacatttaaagaagacatccaaaaggcccaagtgagtgggcaaggtgattcattacatgaacatattgcaaatttagccggatctcctgctattaagaaagggatattacaaactgttaaagttgtggatgaattagtgaaagtaatgggaagacataaacctgaaaacattgtcattgagatggcaagagaaaatcaaactacacaaaaaggacagaaaaatagtagagaacgtatgaaaagaatagaagagggtattaaagaattgggtagtcaaatattgaaagaacacccagtggaaaatacccagttgcaaaatgaaaaattatatctttactaccttcaaaatggacgtgatatgtatgttgatcaggaattagatataaatagactttcagattatgatgtagatgcaatagttccacaatctttcttgaaagatgattccatagacaataaagtattaactagaagtgataaaaatagaggtaaaagtgataatgtcccaagtgaggaagtcgtcaaaaagatgaaaaattactggcgtcaacttttgaatgctaaattaattactcaaagaaaatttgataatttgactaaagcagaaagaggtgggctttctgaattagataaagccgggttcattaaaagacaattggtcgaaactagacaaattactaaacatgttgcccaaattttagattcccgtatgaacactaagtatgacgaaaatgataagttaatacgtgaggttaaagtcattactttaaaatcaaaacttgtctctgatttcagaaaggatttccaattctataaagttagagaaattaataattatcatcatgctcatgatgcatatttgaatgctgtagttggaactgctttaatcaagaaataccctaaattagaatctgaatttgtatatggtgattacaaagtctatgatgttagaaagatgattgctaaatcagaacaagaaattggtaaagctacagctaaatacttcttttactctaacattatgaatttctttaaaacagaaattactttggcaaacggtgaaattagaaaaagacctcttattgaaacaaatggtgagactggagagatagtttgggacaaagggcgtgatttcgctactgttagaaaagttttatcaatgccacaagttaacattgtaaagaaaacagaggttcaaactggtggtttctcaaaagaaagtattttgcctaaaagaaatagtgataaattgattgccagaaaaaaggattgggatccaaagaaatatggtggtttcgactcaccaaccgtagcctattctgttttggttgtggcaaaggttgaaaagggtaaaagtaaaaagcttaaatcagtaaaagaacttttgggtattacaataatggaaagaagttcctttgaaaagaaccctattgattttttggaagctaaaggttataaggaagtaaagaaggacttaataatcaaattgcctaaatattctttatttgaattagaaaatgggagaaaaagaatgttggcttctgctggagaattgcaaaagggtaatgaattagcattgccttccaaatatgttaacttcttgtatttagcttcacactatgaaaagttgaaagggtcaccagaagataacgagcaaaaacaattatttgttgaacaacacaaacactacttagatgagattatagaacaaattagtgaattcagtaaaagagtgatattagctgatgcaaatttagataaagttttgtcagcctataacaaacatagagataagccaattagagaacaagcagaaaacattattcacttatttacccttaccaatttaggagcacctgctgctttcaagtattttgatacaacaattgatcgtaaaagatatacctcaacaaaagaagtcttagacgccaccttaattcatcaatcaatcactggattgtatgagacaagaattgatttgtctcaattgggtggtgatgaaggggct

SEQ ID NO: 30 - protein, 1080 aa, synthetic construct
MYATAHTIKQQQQQQQQHPPPPLNGGLHASGAPPNSHEAAAIAQQQQQQQQHHNGPGMIVAAAAASANQQAVQARAQQQQQQQQQRLPSSAALNETTVSTWLAIGSLAESLGDIERATASYNSALRHSPNNPDILVKIANTYRSKDQFLKAAELYEQALNFHVENGETWGLLGHCYLMLDNLQRAYAAYQRALFYLENPNVPKLWHGIGILYDRYGSLEYAEEAFVRVLDLDPNFDKANEIYFRLGIIYKHQGKLQPALECFQYILNNPPHPLTQPDVWFQIGSVYEQQKDWNGAKDAYEKVLQINPHHAKVLQQLGCLYSQAESNPSTPANGAAPPHKPFQQDLTIALKYLKQSLEVDQSDAHSWYYLGRVEMIRGDFTAAYEAFQQAVNRDARNPTFWCSIGVLYYQISQYRDALDAYTRAIRLNPYISEVWYDLGTLYETCNNQISDALDAYRQAERLDPNNPHIKARLEQLTKYQQEGNTHPPQPPPSSQQPRLPQGMVLESTQQQQQQQPPPPPQQQQQQLQHQSQSQPQPQQPPQTQSQPSLLQHQSSLPPQQIQPLHQQAAKPLVNQQQSPPPPHLMNLGQPGQQPQQLPPHLPPHTQQPSQIQEKPPTQEQPHYQPPPPPQHQQQSQSQPQPPHQPQHTQNQSPQLAQLPPHHSNPPAKPHGAPQQRTGLPDLLHNSANIISAPSQVPQPQQQYQQPHIAPVRQEQVNHVPSIYSAPRPTETTLPQINNPNESTTTQVPQLKKEEPKPEATVSAPVPEAIKVQDQVTIQESAPAAAAAVSAPASAPVGDIKTDTVSTTTPATSTTADAVPVSVSQVGEAPNVVQEKKVPDTEQIVSQVEKPVESQPEVTPAPTPAPALATAPTEPAPTDKDVVMAPSKSATPVPQSIVEQNTRVSEATKAPESNGKHDLEDKNDEEKILKRPTVETTTESVPVNQPVEKENEKVEVPPPSEQPSSEKREKEVNGSIKKPLENESKVDIPQFSSNITAQNEEAKSGEETKKDTTKTSPAKQGEVKEVIPSSTETVSKPDVEKDNKEKDKDEDEVMADEDDVKKDENPEPPMRKIEEDENYDDE

SEQ ID NO: 31 - protein, 11 aa, synthetic construct
DALDDFDLDML

SEQ ID NO: 32 - DNA, 144 bp, synthetic construct
  source 1..144: mol_type = other DNA
gatcctaagaagaaaagaaaagttgatccaaagaaaaagcgtaaggtggatcctaagaaaaagagaaaggttgactacaaagaccatgacggtgattataaagatcatgacatcgactacaaggatgacgatgacaagtgataa

SEQ ID NO: 33 - protein, 32 aa, Candida albicans
SIISIIMGILTNIPQVIQIIMSIVRAFKGNKR

SEQ ID NO: 34 - protein, 32 aa, Candida albicans
SIISIIMGILSNIPQVIQIIMSIVRAFKGNKR

SEQ ID NO: 35 - protein, 32 aa, Candida albicans
SIIGIIMGILGNIPQVIQIIMSIVKAFKGNKR

SEQ ID NO: 36 - protein, 32 aa, Candida albicans
SFLSIITALLGNIPQIIQIIMGIVKAFRGNKR